(19) United States
(12) Patent Application Publication
(10) Pub. No.: US 2024/0050454 A1
(43) Pub. Date: Feb. 15, 2024

(54) Compositions and Methods For Treating Neurological Conditions

(71) Applicants: Jeffrey Jewell, Chicago, IL (US); Mark Wingertzahn, Chicago, IL (US); WeSana Health Inc., Chicago, IL (US)

(72) Inventors: Jeffrey Jewell, Chicago, IL (US); Mark Wingertzahn, Chicago, IL (US); Daniel Carcillo, Chicago, IL (US)

(21) Appl. No.: 18/254,705

(22) PCT Filed: Nov. 30, 2021
(86) PCT No.: PCT/US2021/061235

§ 371 Date: May 26, 2023

Related U.S. Application Data
(60) Provisional application No. 63/119,529, filed on Nov. 30, 2020.

(51) Int. Cl. A61K 31/675; A61K 36/07; A61K 31/00; A61K 45/06; A61K 36/9066; A61K 36/185; A61K 31/55; A61P 25/24

(52) U.S. Cl. CPC A61K 31/675; A61K 36/07; A61K 31/658; A61K 45/06; A61K 36/9066; A61K 36/185; A61K 31/55; A61P 25/24

(57) ABSTRACT

As described below, the present disclosure features compositions containing a cannabidiol (CBD) and/or psilocybin, optionally in combination with a mushroom blend. The present disclosure also features methods for treating neurological conditions (e.g., traumatic brain injury or TBI), or symptoms thereof (e.g., anxiety or depression), with a cannabidiol and/or psilocybin, optionally in combination with a mushroom blend. The present disclosure also features compositions and methods of using psilocybin in combination with a neurotransmitter activity modulator for the treatment of neurological conditions (e.g., TBI), or symptoms thereof (e.g., anxiety or depression).

### CROSS-REFERENCE TO RELATED APPLICATION

This application claims the benefit of the following U.S. Provisional Application No. 63/119,529, filed Nov. 30, 2020, the entire contents of which are incorporated herein by reference.

### BACKGROUND

Traumatic brain injury (TBI) is typically associated with a violent blow, bump, or jolt to the head. TBI can occur when the head suddenly and violently hits an object, or when an object pierces the skull and enters brain tissue. Clinical signs include loss of or decreased consciousness; amnesia; focal neurological deficits, such as muscle weakness, loss of vision, and/or change in speech; and alteration in mental state, such as disorientation, slow thinking, poor memory, or difficulty concentrating. TBI can cause one or more mass lesions, e.g., areas of localized injury, such as hematomas and contusions. Other sequelae which may develop from TBI include intracerebral hemorrhage, subarachnoid hemorrhage, diffuse injuries (microscopic changes that do not appear on CT scans and which may be scattered throughout the brain), diffuse axonal injury, ischemia, and skull fracture. Mild TBI, sometimes called concussion, may not require specific treatment other than rest. However, severe TBI can require emergency care. Emergency care generally focuses on stabilizing and keeping the patient alive, including making sure the brain gets enough oxygen, controlling blood and brain pressure, and preventing further injury to the head or neck. Once the patient is stable, other types of care for TBI can begin. Sometimes surgery is needed as part of emergency care to reduce damage to the brain. Surgery may removing blood clots or pools; repairing skull fractures or relieving pressure inside the skull. Typically, TBI is treated by surgical means to remove resultant hematomas, contusions, and/or lesions. Medications to prevent further nerve damage or promote nerve healing for faster recovery is not currently available.

Concussion is a form of mild traumatic brain injury that usually happens after a blow to the head. It can also occur with violent shaking and movement of the head or body. Symptoms associated with concussion may include headaches, dizziness, and problems with concentration and memory. The usual recovery period for patients suffering a concussion is weeks to months. Unfortunately, some subjects fail to recover within the normal time period. Such subjects are said to be suffering from post-concussion syndrome, which occurs when concussion symptoms last beyond the expected recovery period after the initial injury. Current methods for treating neurological conditions, such as traumatic brain injury (TBI), post-concussion syndrome, post-traumatic headache, progressive headache, and symptoms thereof are inadequate. Accordingly, improved methods for treating such conditions are urgently required.

### SUMMARY

As described below, the present disclosure features compositions comprising a psilocybin and/or cannabidiol (CBD) and/or psilocin, optionally in combination with a mushroom blend. Other embodiments include compositions comprising psilocybin or a derivative thereof, in combination with a neurotransmitter activity modulator (e.g., serotonin, norepinephrine, histaminergic, muscarinic, and dopaminergic). The present disclosure also features methods for treating traumatic brain injury (TBI) using compositions described herein. Other neurological conditions (e.g., anxiety, depression, stress, post-concussion syndrome, post-traumatic headache, and/or progressive headache) or symptoms thereof, are also treatable with the methods described herein. In some embodiments, the treatments feature, e.g., a regimen of psilocybin and/or cannabidiol and/or psilocin, optionally in combination with a mushroom blend. In other embodiments, the treatments feature a regimen of psilocybin or a derivative thereof, in combination with a neurotransmitter activity modulator.

Presented herein are methods for reducing a symptom of traumatic brain injury or post-concussion syndrome in a subject, the method comprising administering to the subject at least one loading dose comprising between about 5 mg and 30 mg psilocybin, inclusive; and subsequently administering to the subject at least one maintenance dose comprising between about 0.25 mg to about 0.3 mg psilocybin, inclusive, wherein the subject has been suspected or diagnosed as having traumatic brain injury or post-concussion syndrome. In some embodiments, the symptom of traumatic brain injury or post-concussion syndrome comprises anxiety, depression, suicide ideation, stress, post-traumatic stress disorder, post-traumatic headache, progressive headache, feelings of dizziness, nausea, vomiting, noise sensitivity, being easily upset by loud noise, sleep disturbance, fatigue or tiring more easily, irritability, insomnia, nervousness, frustration or impatience, forgetfulness or poor memory, poor concentration, taking longer to think, blurred vision, light sensitivity, double vision, restlessness, insomnia, ringing in the ears, blurry vision, decrease in taste and/or smell, and various combinations thereof. In some embodiments, the at least one maintenance dose further comprises between about 100 mg and about 600 mg CBD, inclusive. In some embodiments, the symptom of traumatic brain injury or post-concussion syndrome comprises anxiety and/or depression. In some embodiments, the loading dose comprises about 10 mg psilocybin. In some embodiments, the loading dose comprises about 25 mg psilocybin. In some embodiments, the loading dose comprises about 30 mg psilocybin. In some embodiments, the psilocybin of the loading dose and/or the maintenance dose comprises synthetic psilocybin. In some embodiments, the psilocybin of the loading dose comprises synthetic psilocybin. In some embodiments, the psilocybin of the maintenance dose comprises synthetic psilocybin. In some embodiments, the synthetic psilocybin is crystalline psilocybin. In some embodiments, the synthetic psilocybin is amorphous psilocybin. In some embodiments, the psilocybin is isolated from the mushroom Psilocybe cubensis. In some embodiments, the CBD comprises synthetic CBD. In some embodiments, the CBD comprises full-spectrum CBD. In some embodiments, the at least one maintenance dose further comprises a composition comprising at least one, at least two, at least three, at least four, at least five, or at least six mushrooms. In some embodiments, the mushroom composition comprising at least one, at least two, at least three, at least four, at least five, or at least six dried mushrooms comprises between about 0.01 g and about 0.5 g of the mushroom composition. In some embodiments, the mushroom composition comprises Hericium erinaceus and Ganoderma lingzhi. In some embodiments, the mushroom composition comprises Laricifomes officinalis, Inonotus obliquus, Cordyceps sp., Hericium erinaceus, Ganoderma lingzhi, Trametes versicolor. In some embodiments, the loading dose is administered prior to an initial maintenance dose by at least seven days. In some embodiments, two or more loading doses are administered over a 6 month time period. In some embodiments, the maintenance dose is administered every day. In some embodiments, the maintenance dose is not administered every day. In some embodiments, the maintenance dose is administered according to a predetermined schedule. In some embodiments, the maintenance dose is administered for five out of every seven days. In some embodiments, the maintenance dose is formulated in a single dosage form. In some embodiments, the maintenance dose is formulated in multiple dosage forms.

Presented herein are compositions for treatment of traumatic brain injury or post-concussion syndrome, the composition comprising: psilocybin, and optionally a dried mushroom blend, wherein the dried mushroom blend comprises at least two mushrooms selected from the group consisting of Laricifomes officinalis, Inonotus obliquus, Cordyceps militaris, Hericium erinaceus, Ganoderma lingzhi, and Trametes versicolor. In some embodiments, the composition further comprises mushrooms belonging to a genus selected from the group consisting of: Agaricus, Schizophyllum, Wolfiporia, Fomes, Pleurotus, Phellinus, Grifola, and Lentinula. In some embodiments, the psilocybin and optionally the dried mushroom blend are formulated together or separately. In some embodiments, the composition further comprises a cannabinoid, niacin, piperine, turmeric, and/or an omega fatty acid. In some embodiments, the composition further comprises a serotonin reuptake inhibitor (SSRI), folic acid, St. John's Wart, dried Codiaeum variegatum, Codiaeum variegatum extract, fish oil, flax seed oil, or combinations thereof.

Presented herein are methods for reducing a symptom of traumatic brain injury or post-concussion syndrome in a subject, the method comprising administering to the subject one or more loading dose(s) of at least about 1 mg psilocybin; and subsequently administering to the subject one or more maintenance dose(s) comprising psilocybin and a dried mushroom blend, wherein the dried mushroom blend comprises at least two mushrooms selected from the group consisting of Laricifomes officinalis, Inonotus obliquus, Cordyceps militaris, Hericium erinaceus, Ganoderma lingzhi, and Trametes versicolor, wherein the maintenance dose comprises less than about 1 mg psilocybin, and wherein the subject has had a traumatic brain injury or has post-concussion syndrome. In some embodiments, the psilocybin is purified and/or synthetic psilocybin. In some embodiments, the loading dose comprises about 1 mg to about 50 mg of purified and/or synthetic psilocybin. In some embodiments, the method further comprises administering to the subject a subsequent loading dose, wherein the subsequent loading dose comprises from about 1 mg to about 50 mg psilocybin. In some embodiments, the subsequent loading dose is separated from the most recently administered loading dose by at least about 1 month. In some embodiments, the maintenance dose is administered about daily. In some embodiments, the maintenance dose is administered for about 3-5 consecutive days followed by no administration for about 1-3 consecutive days. In some embodiments, the maintenance dose is administered daily followed by no administration for 2 consecutive days. In some embodiments, the maintenance dose further comprises turmeric and piperine. In some embodiments, the maintenance dose further comprises cannabidiol. In some embodiments, the maintenance dose comprises from about 0.05 to about 1 mg psilocybin. In some embodiments, the maintenance dose comprises from about 5 mg to about 100 mg cannabidiol. In some embodiments, wherein the dried mushroom blend comprises from about 1 wt % to about 60 wt % by dry weight of Inonotus obliquus, from about 0.3 wt % to about 8 wt % by dry weight of Agaricus subrufescens, from about 3 wt % to about 60 wt % by dry weight of Laricifomes officinalis, from about 3 wt % to about 60 wt % by dry weight of Trametes versicolor, from about 3 wt % to about 60 wt % by dry weight of Ganoderma lingzhi, from about 0.3 wt % to about 8 wt % by dry weight of Schizophyllum commune, from about 3 wt % to about 60 wt % by dry weight of Hericium erinaceus, from about 0.3 wt % to about 8 wt % by dry weight of Wolfiporia extensa, from about 3 wt % to about 60 wt % by dry weight of Cordyceps militaris, from about 0.3 wt % to about 8 wt % by dry weight of Fomes fomentarius, from about 0.3 wt % to about 8 wt % by dry weight of Pleurotus ostreatus, from about 0.3 wt % to about 8 wt % by dry weight of Phellinus linteus, from about 0.3 wt % to about 8 wt % by dry weight of Grifola frondosa, and from about 0.3 wt % to about 8 wt % by dry weight of Lentinula edodes. In some embodiments, the wherein the psilocybin, the cannabidiol, and/or the dried mushroom blend are formulated together or separately. In some embodiments, the psilocybin, the cannabidiol, and/or the dried mushroom blend are administered concurrently or substantially concurrently. In some embodiments, the psilocybin, the cannabidiol, and/or the dried mushroom blend are administered within hours or days of one another. In some embodiments, the maintenance dose further comprises niacin, piperine, turmeric, and/or omega fatty acid. In some embodiments, the maintenance dose further comprises an agent selected from the group consisting of a serotonin reuptake inhibitor (SSRI), folic acid, St. John's Wart, dried Codiaeum variegatum, Codiaeum variegatum extract, fish oil, and flax seed oil. In some embodiments, the subject has post-concussion syndrome. In some embodiments, the subject has had a traumatic brain injury. In some embodiments, the maintenance dose and/or loading dose comprises the mushroom Psilocybe cubensis.

Provided herein are methods for reducing a symptom of traumatic brain injury or post-concussion syndrome in a subject, the method comprising administering to the subject at least one loading dose comprising between about 5 mg and about 30 mg psilocybin, inclusive; and subsequently administering to the subject at least one maintenance dose comprising between about 1 mg to about 600 mg of a neurotransmitter activity modulator, and wherein the subject has been suspected or diagnosed as having traumatic brain injury or post-concussion syndrome. In some embodiments, the symptom of traumatic brain injury or post-concussion syndrome comprises anxiety, depression, suicide ideation, stress, post-traumatic stress disorder, post-traumatic headache, progressive headache, feelings of dizziness, nausea, vomiting, noise sensitivity, being easily upset by loud noise, sleep disturbance, fatigue or tiring more easily, irritability, insomnia, nervousness, frustration or impatience, forgetfulness or poor memory, poor concentration, taking longer to think, blurred vision, light sensitivity, double vision, restlessness, insomnia, ringing in the ears, blurry vision, decrease in taste and/or smell, and various combinations thereof. In some embodiments, the symptom of traumatic brain injury or post-concussion syndrome comprises anxiety and/or depression. In some embodiments, the loading dose comprises about 5 mg to about 15 mg psilocybin. In some embodiments, the loading dose comprises about 8 mg to about 12 mg psilocybin. In some embodiments, the loading dose comprises about 25 mg psilocybin. In some embodiments, the loading dose comprises about 30 mg psilocybin. In some embodiments, the at least one maintenance dose further comprises between about 0.25 mg and about 0.3 mg psilocybin. In some embodiments, the psilocybin of the loading dose comprises synthetic psilocybin. In some embodiments, the psilocybin of the maintenance dose comprises synthetic psilocybin. In some embodiments, the synthetic psilocybin is crystalline psilocybin. In some embodiments, the synthetic psilocybin is amorphous psilocybin. In some embodiments, the psilocybin is isolated from the mushroom Psilocybe cubensis. In some embodiments, the neurotransmitter activity modulator comprises one or more selected from the group consisting of a tricyclic antidepressant (TCA), a selective serotonergic reuptake inhibitor (SSRI), a serotonin-norepinephrine reuptake inhibitor (SNRI), and a norepinephrine reuptake inhibitor (NRI). In some embodiments, the neurotransmitter activity modulator comprises a tricyclic antidepressant (TCA). In some embodiments, the neurotransmitter activity modulator comprises an SSRI. In some embodiments, the neurotransmitter activity modulator comprises an SNRI. In some embodiments, the neurotransmitter activity modulator comprises an NRI. In some embodiments, the neurotransmitter activity modulator comprises imipramine. In some embodiments, the loading dose is administered prior to an initial maintenance dose by at least seven days. In some embodiments, two or more loading doses are administered over a 6 month time period. In some embodiments, the maintenance dose is administered every day. In some embodiments, the maintenance dose is not administered every day. In some embodiments, the maintenance dose is administered according to a predetermined schedule.

Presented herein are methods for reducing a symptom of traumatic brain injury or post-concussion syndrome in a subject, the method comprising administering to the subject at least one maintenance dose comprising between about 0.25 mg and about 0.3 mg psilocybin, inclusive; and administering to the subject at least one maintenance dose comprising between about 1 mg to about 600 mg of CBD, inclusive, wherein the subject has been suspected or diagnosed as having traumatic brain injury or post-concussion syndrome. In some embodiments, the symptom of traumatic brain injury or post-concussion syndrome comprises anxiety, depression, suicide ideation, stress, post-traumatic stress disorder, post-traumatic headache, progressive headache, feelings of dizziness, nausea, vomiting, noise sensitivity, being easily upset by loud noise, sleep disturbance, fatigue or tiring more easily, irritability, insomnia, nervousness, frustration or impatience, forgetfulness or poor memory, poor concentration, taking longer to think, blurred vision, light sensitivity, double vision, restlessness, insomnia, ringing in the ears, blurry vision, decrease in taste and/or smell, and various combinations thereof. In some embodiments, the symptom of traumatic brain injury or post-concussion syndrome comprises anxiety or depression. In some embodiments, the symptom of traumatic brain injury or post-concussion syndrome comprises anxiety. In some embodiments, the symptom of traumatic brain injury or post-concussion syndrome comprises depression. In some embodiments, the maintenance dose is administered every day. In some embodiments, the maintenance dose is administered according to a predetermined schedule. In some embodiments, the loading dose comprises synthetic psilocybin. In some embodiments, the synthetic psilocybin is crystalline psilocybin. In some embodiments, the synthetic psilocybin is amorphous psilocybin. In some embodiments, the psilocybin is isolated from the mushroom Psilocybe cubensis. In some embodiments, the neurotransmitter activity modulator comprises one or more selected from the group consisting of a tricyclic antidepressant (TCA), a selective serotonergic reuptake inhibitor (SSRI), a serotonin-norepinephrine reuptake inhibitor (SNRI), and a norepinephrine reuptake inhibitor (NRI). In some embodiments, the neurotransmitter activity modulator comprises a tricyclic antidepressant (TCA). In some embodiments, the neurotransmitter activity modulator comprises an SSRI. In some embodiments, the neurotransmitter activity modulator comprises an SNRI. In some embodiments, the neurotransmitter activity modulator comprises an NRI. In some embodiments, the neurotransmitter activity modulator comprises imipramine.

Presently described herein are compositions comprising psilocybin and a neurotransmitter activity modulator from the group consisting of a tricyclic antidepressant, an SSRI, an SNRI, and an NRI. In some embodiments, the neurotransmitter activity modulator comprises a tricyclic antidepressant. In some embodiments, the neurotransmitter activity modulator comprises imipramine. In some embodiments, the neurotransmitter activity modulator comprises an SSRI. In some embodiments, the neurotransmitter activity modulator comprises an SNRI. In some embodiments, the neurotransmitter activity modulator comprises an NRI. In some embodiments, the neurotransmitter activity modulator comprises about 1 mg to about 600 mg of the neurotransmitter activity modulator. In some embodiments, the neurotransmitter activity modulator comprises about 1 mg to about 300 mg of the neurotransmitter activity modulator. In some embodiments, the neurotransmitter activity modulator comprises about 1 mg to about 200 mg of the neurotransmitter activity modulator. In some embodiments, the neurotransmitter activity modulator comprises about 1 mg to about 100 mg of the neurotransmitter activity modulator. In some embodiments, the neurotransmitter activity modulator comprises about 1 mg to about 50 mg of the neurotransmitter activity modulator. In some embodiments, the psilocybin comprises about 0.1 mg to about 0.5 mg. In some embodiments, the psilocybin comprises about 0.25 mg to about 0.3 mg.

Other features and advantages of the disclosure will be apparent from the detailed description, and from the claims.

### Definitions

Unless defined otherwise, all technical and scientific terms used herein have the meaning commonly understood by a person skilled in the art to which this disclosure belongs. As used herein, the following terms have the meanings ascribed to them below, unless specified otherwise.

By “cannabinoid” is meant a group of chemicals known to activate cannabinoid receptors in cells. In various embodiments, cannabinoids are obtained from Cannabis plants. In some embodiments, the cannabinoid is synthetic. In one embodiment, the cannabinoid is endogenous to an animal, i.e., an endocannabinoid. In one embodiment, the cannabinoid is derived from a plant, e.g., a plant of genus Cannabis, i.e., a phytocannabinoid. There are at least 113 different cannabinoids isolated from Cannabis, exhibiting varied (similar and different) effects. Non-limiting examples of cannabinoids include the following molecules: Cannabichromene (CBC), Cannabichromenic acid (CBCA), Cannabichromevarin (CBCV), Cannabichromevannic acid (CBCVA), Cannabicyclol (CBL), Cannabicyclolic acid (CBLA), Cannabicyclovarin (CBLV), Cannabidiol (CBD), Cannabidiol monomethylether (CBDM), Cannabidiolic acid (CBD A), Cannabidiorcol (CBD-C1), Cannabidivarin (CBDV), Cannabidivarinic acid (CBDVA), Cannabielsoic acid B (CBEA-B), Cannabielsoin (CBE), Cannabielsoin acid A (CBEA-A), Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA), Cannabinodiol (CBND), Cannabinodivarin (CBDV), Cannabinol (CBN), Cannabinol methylether (CBNM), Cannabinol-C2 (CBN-C2), Cannabinol-C4 (CBN-C4), Cannabinolic acid (CBN A), Cannabiorcool (CBN-C1), Cannabivarin (CBV), Cannabitriol (CBT), Cannabitriolvarin (CBTV), 10-Ethoxy-9-hydroxy-delta-6a-tetrahydrocannabinol, Cannbicitran (CBT), Cannabiripsol (CBR), 8,9-Dihydroxy-delta-6a-tetrahydrocannabinol, Delta-8-tetrahydrocannabinol (AS-THC), Delta-8-tetrahydrocannabinolic acid (AS-THCA), Delta-9-tetrahydrocannabinol (THC), Delta-9-tetrahydrocannabinol-C4 (THC-C4), Delta-9-tetrahydrocannabinolic acid A (THC A-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV), Delta-9-tetrahydrocannabivarinic acid (THCVA), 10-Oxo-delta-6a-tetrahydrocannabinol (OTHC), Cannabichromanon (CBCF), Cannabifuran (CBF), Cannabiglendol, Delta-9-cis-tetrahydrocannabinol (cis-THC), Tryhydroxy-delta-9-tetrahydrocannabinol (triOH-THC), Dehydrocannabifuran (DCBF), and 3,4,5,6-Tetrahydro-7-hydroxy-alpha-alpha-2-trimethyl-9-n-propyl-2,6-methano-2H-1-benzoxocin-5-methanol. In one embodiment, the term “cannabinoid” refers to a compound chosen from THC, THCA, THCV, THCVA, CBC, CBCA, CBCV, CBCVA, CBD, CBDA, CBDV, CBDVA, CBG, CBGA, CBGV, or CBGVA. In some embodiments, the cannabinoid is cannabidiol (CBD). In some embodiments, the cannabinoid is a nanoparticulate, for example a nanoparticulate CBD. In some embodiments, the cannabinoid is in the form of a Cannabis plant extract, optionally wherein the plant extract is free of THC (tetrahydrocannabinol) or THCA (tetrahydrocannabinolic acid).

By “cannabidiol (CBD)” is meant 2-[(6R)-3-methyl-6-prop-1-en-2-ylcyclohex-2-en-1-yl]-5-pentylbenzene-1,3-diol having CAS number 13956-29-1 and the structure,

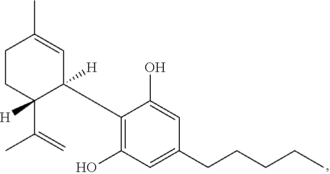 [SMILES: C=C(C)[C@@H]1CCC(C)=C[C@H]1Cc1c(O)cc(CCCCC)cc1O]

and pharmaceutically acceptable salts thereof. In some embodiments, cannabidiol is synthetic.

By “psilocybin” is meant [3-(2-dimethylaminoethyl)-1H-indol-4-yl] dihydrogen phosphate, which has the molecular formula C₁₂H₁₇N₂O₄P, having the CAS number 520-52-5 and the structure,

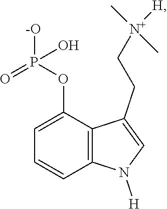

and pharmaceutically acceptable salts thereof. In some embodiments, psilocybin is synthetic. In some embodiments, the psilocybin is amorphous psilocybin. In other embodiments, psilocybin is extracted from a biological sample.

By “psilocin” is meant 4-hydroxy-N,N-dimethyltryptamine, having the CAS number 520-53-6 and the structure,

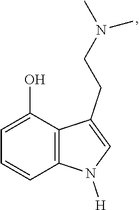

and pharmaceutically acceptable salts thereof. In some embodiments, “psilocin” is alternatively referred to as 4-HO-DMT, psilocine, psilocyn, or psilotsin. In some embodiments, the psilocin is synthetic.

In one embodiment, a psilocybin derivative within the compositions disclosed herein is a compound defined by the following structural formula A:

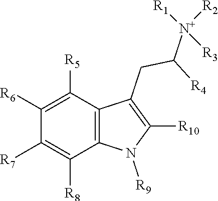

where each of R₁, R₂, R₃, R₄, R₅, R₆, R₇, R₈, R₉, and R₁₀ is chosen from an electron pair, a hydrogen, an alkyl, an alkenyl, an alkynyl, a phenyl, a halide, a hydroxyl, a carbonyl, an aldehyde, a haloformyl, a carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, or a phosphate.

In one embodiment, a psilocybin derivative is a compound defined by the structural formula A, wherein each of R₁, R₂, and R₃ is independently chosen from an electron pair, a hydrogen, or an alkyl; wherein R₄ is hydrogen; wherein R₅ is chosen from a hydroxyl, an ether, or a phosphate; and wherein each of R₆ R₇, R₈, R₉, and R₁₀ is hydrogen.

In one embodiment, a psilocybin derivative is a compound defined by the structural formula A, wherein each of R₁, R₂, and R₃ is independently chosen from an electron pair, a hydrogen, or an alkyl; wherein each of R₄ and R₅ is hydrogen; wherein R₈ is chosen from a hydroxyl, an ether, or a phosphate; and wherein each of R₇, R₈, R₉, and R₁₀ is hydrogen.

Unless otherwise specified, any of the compounds of the present disclosure may be, in various embodiments, present in its protonated or deprotonated (salt or freebase) forms or mixtures thereof. The form of a compound may depend on context, for example the pH of the solution or composition. Further, unless otherwise specified, a pharmaceutically acceptable salt of any of the compounds discussed herein is contemplated as being suitable for use in the compositions, methods, and/or dosage forms of the present disclosure.

In one embodiment, a psilocybin derivative is “4-hydroxy-N,N,N-trimethyltryptamine”, which refers to a compound, and/or salts thereof, with the following structural formula:

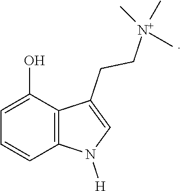 [SMILES: C[N+](C)(C)CCc1c[nH]c2cccc(O)c12]

In one embodiment, a psilocybin derivative is “[3-(2-methylaminoethyl)-1H-indol-4-yl]dihydrogen phosphate”, which refers to a compound, and/or salts thereof, with the following structural formula:

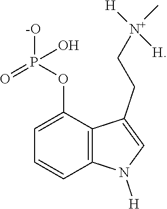

In one embodiment, a psilocybin derivative is “4-hydroxy-N-methyltryptamine”, which refers to a compound, and/or salts thereof, with the following structural formula:

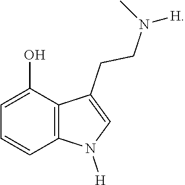

In one embodiment, a psilocybin derivative is “[3-(aminoethyl)-1H-indol-4-yl]dihydrogen phosphate”, which refers to a compound, and/or salts thereof, with the following structural formula:

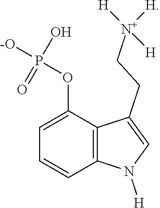

In one embodiment, a psilocybin derivative is “4-hydroxytryptamine”, which refers to a compound, and/or salts thereof, with the following structural formula:

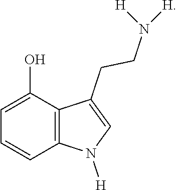

As used herein, the term “THC”, or tetrahydrocannabinol, refers to a compound with the following structural formula:

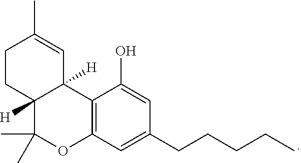 [SMILES: CCCCCc1cc(O)c2c(c1)OC(C)(C)[C@@H]1CCC(C)=C[C@@H]21]

As used herein, the term “THCA” refers to a compound with the following structural formula:

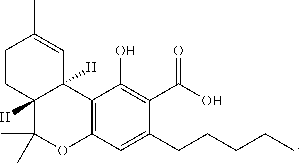 [SMILES: CCCCCc1cc2c(c(O)c1C(=O)O)[C@@H]1C=C(C)CC[C@H]1C(C)(C)O2]

As used herein, the term “THCV” refers to a compound with the following structural formula:

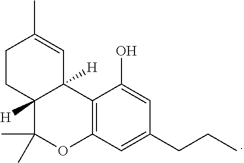 [SMILES: CCCc1cc(O)c2c(c1)OC(C)(C)[C@@H]1CCC(C)=C[C@@H]21]

As used herein, the term “THCVA” refers to a compound with the following structural formula:

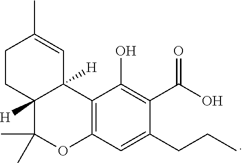 [SMILES: CCCc1cc2c(c(O)c1C(=O)O)[C@@H]1C=C(C)CC[C@H]1C(C)(C)O2]

As used herein, the term “CBC” refers to a compound with the following structural formula:

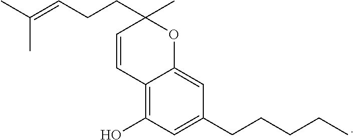 [SMILES: CCCCCc1cc(O)c2c(c1)OC(C)(CCC=C(C)C)C=C2]

As used herein, the term “CBCA” refers to a compound with the following structural formula:

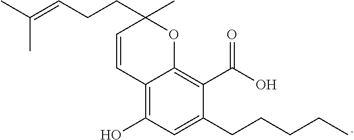 [SMILES: CCCCCc1cc(O)c2c(c1C(=O)O)OC(C)(CCC=C(C)C)C=C2]

As used herein, the term “CBCV” refers to a compound with the following structural formula:

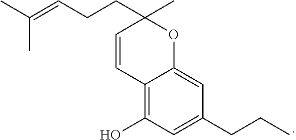 [SMILES: CCCc1cc(O)c2c(c1)OC(C)(CCC=C(C)C)C=C2]

As used herein, the term “CBCVA” refers to a compound with the following structural formula:

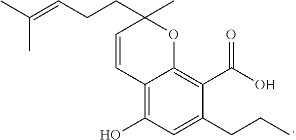 [SMILES: CCCc1cc(O)c2c(c1C(=O)O)OC(C)(CCC=C(C)C)C=C2]

As used herein, the term “CBDA” refers to a compound with the following structural formula:

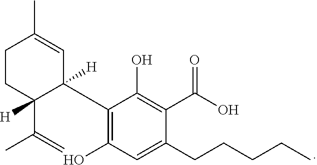 [SMILES: C=C(C)[C@@H]1CCC(C)=C[C@H]1c1c(O)cc(CCCCC)c(C(=O)O)c1O]

As used herein, the term “CBDV” refers to a compound with the following structural formula:

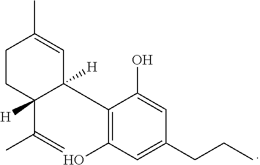 [SMILES: C=C(C)[C@@H]1CCC(C)=C[C@H]1c1c(O)cc(CCC)cc1O]

As used herein, the term “CBDVA” refers to a compound with the following structural formula:

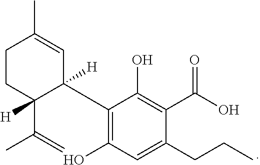 [SMILES: C=C(C)[C@@H]1CCC(C)=C[C@H]1c1c(O)cc(CCC)c(C(=O)O)c1O]

As used herein, the term “CBG” refers to a compound with the following structural formula:

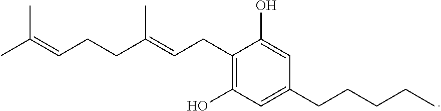 [SMILES: CCCCCc1cc(O)c(C/C=C(\C)CCC=C(C)C)c(O)c1]

As used herein, the term “CBGA” refers to a compound with the following structural formula:

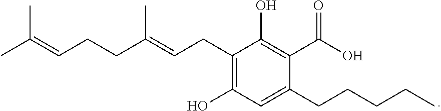 [SMILES: CCCCCc1cc(O)c(C/C=C(\C)CCC=C(C)C)c(O)c1C(=O)O]

As used herein, the term “CBGV” refers to a compound with the following structural formula:

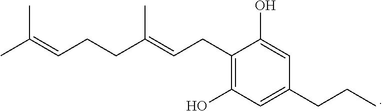 [SMILES: CCCc1cc(O)c(C/C=C(\C)CCC=C(C)C)c(O)c1]

As used herein, the term “CBGVA” refers to a compound with the following structural formula:

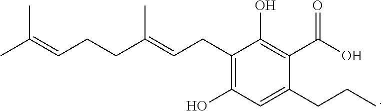 [SMILES: CCCc1cc(O)c(C/C=C(\C)CCC=C(C)C)c(O)c1C(=O)O]

The term “administer” can refer to dosing, treating, giving, or providing. Administering or administration can refer to one particular administration event. In other embodiments, administering or administration can refer to an administration protocol, wherein parts of the composition are provided at intervals, wherein administering can refer to a part or the whole of the treatment protocol.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change in a measured quantity. The change can be an increase or a decrease. As used herein, an alteration includes a 10% change, a 25% change, a 40% change, a 50% change, a 60% change, a 70% change, an 80% change, a 90% change, or a 95% or greater change.

In this disclosure, “comprises,” “comprising,” “containing,” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

By “derivative” is meant a compound retaining a physiological activity of an original compound. In some embodiments, the derivative of a compound has substantially the same or improved activity relative to the starting compound. The derivative may be a metabolite of the original compound from which it is derived. The derivative may be produced in some embodiments according to standard principles of medicinal chemistry. In some embodiments, the derivative may exhibit a lesser degree of activity than the starting material, so long as sufficient activity is retained as to be therapeutically effective. The derivative of a compound may exhibit improved solubility, reduced toxicity, improved potency, enhanced uptake, and the like. The derivative can be a derivative of a cannabinoid or of psilocybin or psilocin. The derivative can be a prodrug form of a compound.

The terms “mushroom blend” and “mushroom composition” are used interchangeably herein. In various embodiments, a mushroom blend comprises dried powder from one or more mushrooms.

The term “dry weight” refers to a measurement of the mass of a sample after removing all, or substantially all, the liquid from the sample. In one embodiment, removing liquid comprises dehydrating, heating, stirring, filtering, and/or any other method suitable for liquid water. In one embodiment, the compositions disclosed herein are in the form of a dried powder. In one embodiment, the compositions disclosed herein comprise between 0-15 mass percent of water as determined by dry weight. In one embodiment, the compositions disclosed herein comprise between 0-10 mass percent of water as determined by dry weight. In one embodiment, the compositions disclosed herein comprise between 0-5 mass percent of water as determined by dry weight. In one embodiment, the compositions disclosed herein comprise between 0-1 mass percent of water as determined by dry weight. In various embodiments, the mass percentages (wt %) referenced throughout the disclosure are determined on a dry-weight basis for a composition.

As used herein, the term “dried powder” refers to a substance composed of fine particles and comprising little or no liquid material. In one embodiment, a dried powder is derived by evaporating alcohol from a solution leaving behind dried particles. In one embodiment, a dried powder is a precipitate from a solution. In one embodiment, a dried powder is a solid collected from a plant (e.g., mushrooms) and pulverized into a powder, e.g., using a mortar and pestle.

By “effective amount” or “therapeutically effective amount” is meant the amount of an agent required to reduce the symptoms of a condition relative to a reference. In one embodiment, a reference is an untreated patient or is the state of the subject at an earlier point in time (e.g., prior to treatment). In some embodiments, the effective amount ameliorates the symptoms of the condition. The effective amount of active compound(s) used to practice the present disclosure for therapeutic treatment of a condition varies depending upon the manner of administration, the age, body weight, and general health of the subject. Ultimately, an attending physician or veterinarian will decide the appropriate amount and dosage regimen. Such amount is referred to as an “effective” amount.

The terms “isolated,” “essentially pure”, “purified,” or “biologically pure” refer to material that is free to varying degrees from components which normally accompany it as found in its native state. In some embodiments, an “essentially pure” compound has a degree of purity of up to at least 90%, 95%, or 99% by weight. In some embodiments “essentially pure” means free from other naturally occurring compounds, such as, other minor cannabinoids and molecules such as terpenes. “Isolate” denotes a degree of separation from original source or surroundings. “Purify” denotes a degree of separation that is higher than isolation. Purity and homogeneity are typically determined using analytical chemistry techniques, for example, high performance liquid chromatography (HPLC) or mass spectroscopy (e.g., GC/MS or LC/MS/MS).

By “neurological condition” is meant a disorder of the brain, spinal cord, or nerves. In some embodiments, the neurological condition is a traumatic brain injury, post-concussion syndrome, anxiety, depression, suicide ideation, stress, post-traumatic headache, and/or progressive headache, and/or symptoms thereof.

As used herein, “obtaining” as in “obtaining an agent” includes synthesizing, purchasing, or otherwise acquiring the agent.

The term “organic” when used with reference to a plant-derived compound is used according to the meaning assigned by the U.S. Department of Agriculture to the term as of November of 2020.

By “pharmaceutically acceptable salt” is meant salts or esters prepared using pharmaceutically acceptable non-toxic bases or acids. In some embodiments, the base or acid includes inorganic bases or acids and/or organic bases or acids.

By “post-concussion syndrome” is meant symptoms associated with a concussion that persist beyond the expected recovery period after. In some embodiments, the traumatic brain injury is a mild or acute traumatic brain injury (mTBI). In various embodiments, the expected recovery period is 1 week, 2, weeks, 3 weeks, 4 weeks, 1 month, 2 months, 3 months, 4 months, 5 months, 6 months, or a year following a concussive injury. In various embodiments, symptoms of post-concussion syndrome include headaches, dizziness, fatigue, irritability, anxiety, insomnia, loss of concentration and/or memory, ringing in the ears, blurry vision, noise and/or light sensitivity, decrease in taste and/or smell, suicide ideation, and various combinations thereof.

By “post-traumatic headache” is meant a headache associated with a traumatic brain injury. A post-traumatic headache can be a headache developing within 1, 2, 3, 4, 5, 6, 7, 8, 9, 10, 11, 12, 13, or 14 days of a traumatic brain injury and/or after regaining consciousness. In embodiments, a post-traumatic headache is a headache developing within 7 days of a traumatic brain injury and/or after regaining consciousness. In embodiments, the post-traumatic headache is a persistent post-traumatic headache, which can be a post-traumatic headache and/or a recurring post-traumatic headache that continues and/or recurs for more than or over about 2 weeks, 1 month, 2 months, 3 months, 4 months, 5 months, or 6 months after a traumatic brain injury. In embodiments, symptoms of a post-traumatic headache include, but are not limited to, pain, pulsing, nausea, vomiting, dizziness, insomnia, trouble concentrating, memory problems, sensitivity to light and/or sound, and/or mood and personality changes, such as depression, suicide ideation, and nervousness. Distinguishing features of post-traumatic headache and methods for diagnosis are known in the art and are described, for example, in Labastida-Ramirez, et al., “Persistent post-traumatic headache: a migrainous loop or not? The clinical evidence”, The Journal of Headache and Pain, vol. 21, article no. 55 (2020), the disclosure of which is incorporated herein by reference in its entirety for all purposes; and in Defrin, “Chronic post-traumatic headache: clinical findings and possible mechanisms”, Journal of Manual and Manipulative Therapy, 22:36-43 (2013), the disclosure of which is incorporated herein by reference.

By “progressive headache” is meant a headache or recurring headache that increases in frequency, duration, and/or severity over time. The progressive headache can be associated with a traumatic brain injury. By “reduces” is meant a negative alteration of at least about 10%, 25%, 50%, 75%, or 100%.

By “reference” is meant a standard or control condition. In some embodiments, the reference is the state of a subject prior to treatment. The state of a subject may include the symptoms experienced prior to treatment. In other embodiments, a reference is the state of an untreated control patient.

By “subject” is meant a mammal. In various embodiments, the mammal is a human or non-human mammal, such as a bovine, equine, canine, ovine, feline, or rodent such as a rat or mouse. As used herein, the terms “patient” and “subject” are used interchangeably. The subject may be male or female. The subject may be a geriatric subject, a pediatric subject, a teenage subject, a young adult subject, or a middle aged subject. In some embodiments, the subject is less than about 18 years of age. In some embodiments, the subject is at least about 18 years of age. In some embodiments, the subject is about 5-10, about 10-15, about 15-20, about 20-25, about 25-30, about 30-35, about 35-40, about 40-45, about 45-50, about 50-55, about 55-60, about 60-65, about 65-70, about 70-75, about 75-80, about 85-90, about 90-95, or about 95-100 years of age.

By “suicide ideation” is meant thoughts about taking one's own life. Suicide ideation can be passive or active. Passive suicidal ideation refers to when one wishes to die or be dead, but without any plans having been constructed to bring about the death. Active suicidal ideation refers to when one wishes to die or be dead and has planned or is planning how to bring about the death. Symptoms of suicide ideation can include self-isolation; feelings of hopelessness; feelings of being trapped; talking about death or suicide; giving away possessions; increase in substance abuse or misuse; increase in mood swings, anger, rage, and/or irritability; engaging in risk-taking behavior like using drugs or having unprotected sex; accessing the means to kill one's self like medications, drugs, or firearms; acting like one is saying goodbye to people; and/or feeling extremely anxious. It is within the skill of a medical practitioner to detect and diagnose suicide ideations in a subject. Suicide ideations can be measured using any method known in the art or provided herein.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. By “traumatic brain injury (TBI)” is meant a disruption in the normal function of the brain associated with a blow to the head. In some embodiments, the traumatic brain injury is a concussion. In some embodiments, the traumatic brain injury results in post-concussion syndrome. In some embodiments, the force to the head includes penetration of the skull and/or brain by an object. In some embodiments, the traumatic brain injury is a concussion. In some embodiments, the physical force is a bump, blow, or jolt to the head, or a penetrating head injury. In some embodiments, the traumatic brain injury is caused by repeated subconcussive head impacts (i.e., head impacts that do not result in symptoms consistent with a diagnosis of concussion). In some embodiments, the traumatic brain injury is caused by multiple concussions. By “subconcussive head impact” is meant a head impact of insufficient force to result in diagnosis of a concussion. In some embodiments, the traumatic brain injury results in disruption of the blood-brain barrier of a subject. In some embodiments, the traumatic brain injury results from about or at least about 1, 2, 3, 4, 5, 6, 7, 8, 9, 10, 11, 12, 13, 14, 15, 16, 17, 18, 19, or 30 instances of a head being subjected to a physical force, optionally wherein each physical force results in a concussion and/or is subconcussive.

As used herein, the terms “treat,” treating,” “treatment,” and the like refer to reducing or ameliorating a condition and/or symptoms associated therewith. It will be appreciated that, although not precluded, treating a disorder or condition does not require that the condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the following Medical Dictionary for Regulatory Activities (MedDRA) terms are considered to be adverse events that are psychedelic in nature: altered mood, altered state of consciousness, autoscopy, delusional perception, disinhibition, dissociation, dissociative identity disorder, dreamy state, emotional disorder, euphoric mood, feeling abnormal, hallucination, hyperacusis, hyperaesthesia, hypoaesthesia, illusion, paranoia, parosmia, photophobia, sensory disturbance, time perception altered, thinking abnormal, synaesthesia, substance-induced psychotic distress, and somatic hallucination. In various embodiments, a maintenance dose of the present disclosure, as described further below, comprises a sufficiently low dose of CBD or a derivative thereof, psilocybin or a derivative thereof, or psilocin or a derivative thereof to avoid or reduce the occurrence of an adverse psychedelic event.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF FIGURES

FIGS. 1A-1G demonstrate that the tested compositions did not have an effect on spontaneous locomotor activity in mice. Specifically, four groups were tested (Group 1=vehicle control; Group 2=psilocybin 0.3 mg/kg+CBD 120 mg/kg+6 mushroom blend 5640 mg/kg; Group 3=psilocybin 0.3 mg/kg, CBD 60 mg/kg+6 mushroom blend 2820 mg/kg; Group 4=psilocybin 0.3 mg+CBD 30 mg/kg+6 mushroom blend 1410 mg/kg). FIG. 1A is a bar graph showing ambulatory distance of the mice of the four groups over the 30 minute test. No statistical differences between groups were observed. FIG. 1B is a bar graph showing ambulatory distance in center of the mice of the four groups over the 30 minute test. No statistical differences between groups were observed. FIG. 1C is a bar graph showing vertical counts of the mice of the four groups over the 30 minute test. No statistical differences between groups were observed. FIG. 1D is a bar graph showing vertical counts in center of the mice of the four groups over the 30 minute test. No statistical differences between groups were observed. FIG. 1E is a bar graph showing average velocity of the mice of the four groups over the 30 minute test. No statistical differences between the groups were observed. FIG. 1F is a bar graph showing average resting time of the mice of the four groups over the 30 minute test. No statistical difference between the groups were observed. FIG. 1G is a bar graph showing average resting time in center of the mice of the four groups over the 30 minute test. No statistical differences between the groups were observed.

FIG. 2 is a bar graph demonstrating that the tested compositions and treatment regimens decrease symptoms of anxiety in mice. Specifically, the bar graph shows that the percentage of time mice spent in an open space was significantly increased when tested two weeks after the mice were administered a loading dose of 5 mg/kg psilocybin.

FIGS. 3A-3C are bar graphs demonstrating that the tested compositions and treatment regimens decrease symptoms of depression in mice across groups at Day 22 post loading dose. Specifically, FIG. 3A is a bar graph showing that a cross-group comparison at Day 22 post loading dose, the amount of time mice spent immobile during a tail suspension test (TST) decreased as the concentration of CBD and a 6 mushroom blend maintenance doses increased.

FIG. 3B is a bar graph showing the same data as in FIG. 3A, wherein the treatment conditions were expressed as a difference from the vehicle condition (expressed as “0”). FIG. 3C is a bar graph showing a percentage improvement from the Day 22 LDP+Vehicle group. LDP=loading dose psilocybin (5 mg/kg); MDP=maintenance dose psilocybin (0.3 mg/kg); LCBD or LDCBD=low dose CBD (30 mg/kg); MCBD or MDCBD=mid dose CBD (60 mg/kg); HCBD or HDCBD (120 mg/kg).

FIGS. 4A-4B are bar graphs showing the “within group” changes in depressive behavior from Day 15 (last day of “LDP only” status) to Day 22 (7 days after adding a maintenance dose of psilocybin+CBD+a six mushroom blend). Specifically, FIG. 4A is a bar graph showing a within group comparison, wherein time spent immobile was documented at Day 15, and at Day 22, seven days after initiation of a high, mid, or low maintenance dose of psilocybin+CBD+a 6 mushroom blend (6 MB) (120:5670 mg/kg, 60:2820, and 30:1410 mg/kg of CBD and the 6 MB, respectively). FIG. 4B is a bar graph showing a percentage improvement from the Day 15 LDP+Vehicle group. LDP=loading dose psilocybin (5 mg/kg); MDP=maintenance dose psilocybin (0.3 mg/kg); LCBD or LDCBD=low dose CBD (30 mg/kg); MCBD or MDCBD=mid dose CBD (60 mg/kg); HCBD or HDCBD (120 mg/kg).

FIG. 5 is a bar graph demonstrating that the tested compositions and treatment regimens decrease symptoms of depression in mice across groups at Day 29 post loading dose. Specifically, FIG. 5 is a bar graph showing that a cross-group comparison at Day 29 post loading dose (fourteen days after initiation of a high, mid, or low maintenance dose of psilocybin+CBD+a 6 mushroom blend (120:5670 mg/kg, 60:2820, and 30:1410 mg/kg of CBD and the 6 MB, respectively), the amount of time spent immobile during a tail suspension test (TST) decreased as compared to vehicle for all groups tested. LDP=loading dose psilocybin (5 mg/kg); MDP=maintenance dose psilocybin (0.3 mg/kg); LCBD or LDCBD=low dose CBD (30 mg/kg); MCBD or MDCBD=mid dose CBD (60 mg/kg); HCBD or HDCBD (120 mg/kg).

FIGS. 6A-6B are bar graphs demonstrating that a loading dose of psilocybin potentiates the anti-depressive properties of the tricyclic antidepressant imipramine. Specifically, FIG. 6A is a bar graph showing that administering a loading dose of psilocybin (5 mg/kg) at Day 1 potentiates the anti-depressive properties of imipramine (15 mg/kg) when administered one hour before tail suspension testing (TST) on Day 22, or Day 29. At Day 22, the group which received a loading dose of psilocybin at Day 1 spent 39% less time immobile than the group which only received imipramine (15 mg/kg) one hour before TST. At Day 29, the group which received a loading dose of psilocybin at Day 1 spent 45% less time immobile than the group which only received imipramine (15 mg/kg) one hour before TST. FIG. 6B is a bar graph showing cross-group changes in depressive behavior at Day 22 for groups that received imipramine (15 mg/kg) at Day 22, a loading dose of psilocybin (5 mg/kg) at Day 1, and a loading dose of psilocybin (5 mg/kg) at Day 1, and imipramine (15 mg/kg) at Day 22.

### DETAILED DESCRIPTION

The disclosure features compositions and methods that are useful for treatment of neurological conditions. In some embodiments, the compositions and methods are useful in the treatment of a neurological condition, such as TBI, anxiety, depression, suicide ideation, stress, post-concussion syndrome, post-traumatic headache, and/or progressive headache.

### Compositions

The compositions of the present disclosure in various embodiments comprise psilocybin or a derivative thereof and/or a cannabinoid, and/or a neurotransmitter activity modulator, optionally in combination with a mushroom blend. In some embodiments, the compositions are pharmaceutical compositions or nutraceutical compositions (e.g., dietary supplements and functional foods) comprising one or more pharmaceutically acceptable additives (e.g., a carrier, excipient, or diluent). The compositions can be used for the treatment of TBI or other neurological condition (e.g., anxiety, depression, suicide ideation, stress, post-concussion syndrome, post-traumatic headache, and/or progressive headache). In some embodiments, one or more compositions described herein can be added to a second composition described herein.

### Psilocybin and Derivatives Thereof

The compositions and methods described herein include psilocybin or a derivative of psilocybin, either of which may be purified from a natural source (e.g., a mushroom plant) or synthetic (i.e., manufactured by artificial means, such as by organic synthesis in a laboratory). In some embodiments, a source of psilocybin in the composition is dried Psilocybe cubensis or a member of the genus Psilocybe (e.g., Psilocybe azurescens, P. semilanceata, or P. cyanescens). In some embodiments, the psilocybin is synthetic psilocybin. In some embodiments, the psilocybin is amorphous psilocybin. In some embodiments the amorphous psilocybin can be sprayed onto a filler or other composition (e.g., a CBD and/or mushroom composition). In some embodiments, the psilocybin is deuterated psilocybin. In some embodiments, the derivative of psilocybin is a metabolite of psilocybin (e.g., psilocin). In some embodiments, the psilocin is naturally extracted. In some embodiments, the psilocin is synthetic. In some embodiments the psilocin is amorphous. In some embodiments, the psilocin is deuterated. In some embodiments the derivative of psilocybin is a prodrug of psilocybin. By “prodrug” is meant a therapeutically inactive compound that can be metabolized in a subject's body to produce a therapeutically active compound.

In various embodiments, the psilocybin derivative is selected from one or more of the following: [3-(2-Dimethylaminoethyl)-1H-indol-4-yl] dihydrogen phosphate; 4-hydroxytryptamine; 4-hydroxy-N,N-dimethyltryptamine; [3-(2-methylaminoethyl)-1H-indol-4-yl] dihydrogen phosphate; 4-hydroxy-N-methyltryptamine; [3-(aminoethyl)-1H-indol-4-yl]dihydrogen phosphate; [3-(2-trimethylaminoethyl)-1H-indol-4-yl] dihydrogen phosphate; and 4-hydroxy-N,N,N-trimethyltryptamine.

In some embodiments, the composition comprises psilocybin in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises psilocybin in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %.

The composition may include synthetic psilocybin or a derivative thereof in an amount of from about 1 mg to about 100 mg. In some embodiments, the psilocybin is synthetic psilocybin. In some embodiments, the psilocybin is crystalline psilocybin. In some embodiments, the psilocybin is amorphous psilocybin. In some embodiments, the psilocybin is deuterated psilocybin. This includes about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 mg, and any amounts therebetween.

The compositions may include synthetic psilocybin or a derivative thereof in an amount from about 1 mg to about 100 mg, about 1 mg to about 90 mg, about 1 mg to about 80 mg, about 1 mg to about 70 mg, about 1 mg to about 60 mg, about 1 mg to about 50 mg, about 1 mg to about 45 mg, about 1 mg to about 40 mg, about 1 mg to about 35 mg, about 1 mg to about 34 mg, about 1 mg to about 33 mg, about 1 mg to about 32 mg, about 1 mg to about 31 mg, about 1 mg to about 30 mg, about 1 mg to about 29 mg, about 1 mg to about 28 mg, about 1 mg to about 27 mg, about 1 mg to about 26 mg, about 1 mg to about 25 mg, about 1 mg to about 24 mg, about 1 mg to about 23 mg, about 1 mg to about 22 mg, about 1 mg to about 21 mg, about 1 mg to about 20 mg, about 1 mg to about 19 mg, about 1 mg to about 18 mg, about 1 mg to about 15 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 45 mg, about 10 mg to about 40 mg, about 10 mg to about 35 mg, about 10 mg to about 34 mg, about 10 mg to about 33 mg, about 10 mg to about 32 mg, about 10 mg to about 31 mg, about 10 mg to about 30 mg, about 10 mg to about 29 mg, about 10 mg to about 28 mg, about 10 mg to about 27 mg, about 10 mg to about 26 mg, about 10 mg to about 25 mg, about 10 mg to about 24 mg, about 10 mg to about 23 mg, about 10 mg to about 22 mg, about 10 mg to about 21 mg, about 10 mg to about 20 mg, about 10 mg to about 19 mg, about 10 mg to about 18 mg, about 10 mg to about 15 mg, about 15 mg to about 90 mg, about 15 mg to about 80 mg, about 15 mg to about 70 mg, about 15 mg to about 60 mg, about 15 mg to about 50 mg, about 15 mg to about 45 mg, about 15 mg to about 40 mg, about 15 mg to about 35 mg, about 15 mg to about 34 mg, about 15 mg to about 33 mg, about 15 mg to about 32 mg, about 15 mg to about 31 mg, about 15 mg to about 30 mg, about 20 mg to about 35 mg, about 21 mg to about 35 mg, about 22 mg to about 35 mg, about 22 mg to about 35 mg, about 23 mg to about 35 mg, about 24 mg to about 35 mg, about 25 mg to about 35 mg, about 20 mg to about 34 mg, about 21 mg to about 34 mg, about 22 mg to about 34 mg, about 23 mg to about 34 mg, about 23 mg to about 33 mg, about 23 mg to about 32 mg, about 23 mg to about 31 mg, about 24 mg to about 35 mg, about 24 mg to about 34 mg, about 24 mg to about 33 mg, about 24 mg to about 32 mg, about 24 mg to about 31 mg, about 24 mg to about 30 mg, about 25 mg to about 35 mg, about 25 mg to about 34 mg, about 25 mg to about 33 mg, about 25 mg to about 32 mg, about 25 mg to about 31 mg, or about 25 mg to about 30 mg psilocybin.

In some embodiments, the synthetic psilocybin or derivative thereof or psilocin or derivative thereof, can be calculated based on the subject's body weight. In some embodiments, the composition comprises about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, or about 1.0 mg/kg of the subject's body weight. In some embodiments, the synthetic psilocybin or a derivative thereof, or psilocin or a derivative thereof can comprise about 0.1 mg/kg to about 1 mg/kg, about 0.2 mg/kg to about 1 mg/kg, about 0.3 mg/kg to about 1 mg/kg, about 0.4 mg/kg to about 1 mg/kg, about 0.1 mg/kg to about 0.9 mg/kg, about 0.1 mg/kg to about 0.8 mg/kg, about 0.1 mg/kg to about 0.7 mg/kg, about 0.1 mg/kg to about 0.6 mg/kg, about 0.1 mg/kg to about 0.5 mg/kg, about 0.1 mg/kg to about 0.4 mg/kg, about 0.2 mg/kg to about 0.8 mg/kg, about 0.3 mg/kg to about 0.7 mg/kg, about 0.3 mg/kg to about 0.6 mg/kg, about 0.3 mg/kg to about 0.5 mg/kg, about 0.3 mg/kg to about 0.45 mg/kg, or about 0.3 mg/kg to about 0.4 mg/kg of the subject's body weight.

The composition may include natural psilocybin or a derivative thereof in an amount of from about 0.5 g to about 6 g. In some embodiments, the psilocybin or derivative there, or the psilocin or derivative thereof is in the form of a dried mushroom, such as Psilocybe cubensis, or dried mushroom blend. In some embodiments, the psilocybin is in the form of natural dried mushroom. In some embodiments, the psilocybin is an extract from natural dried mushrooms.

This includes about 0.5 g, about 0.6 g, about 0.7 g, about 0.8 g, about 0.9 g, about 1.0 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, about 5.0 g, about 5.2 g, about 5.4 g, about 5.6 g, about 5.8 g, about 6.0 g. The compositions may include psilocybin or a derivative thereof in an amount from about 0.5 g mg to about 6 g, about 1 g to about 6 g, about 1 g to about 5 g, about 1 g to about 4 g, about 1 g to about 3 g, about 1 g to about 2 g, about 2 g to about 6 g, about 2 g to about 5 g, about 2 g to about 4 g, about 2 g to about 3 g, about 3 g to about 6 g, about 3 g to about 5 g, about 3 g to about 4 g, about 4 g to about 6 g, about 4 g to about 5 g. In some embodiments, the amount of psilocybin or derivative thereof or psilocin or derivative thereof is about 5 g of dried mushrooms or a dried mushroom blend.

The composition may include psilocybin or a derivative thereof, or psilocin or a derivative thereof is in the form of a dried mushroom, such as Psilocybe cubensis, or dried mushroom blend. In some embodiments, the psilocybin is an extract from natural dried mushrooms. The amount of Psilocybe cubensis, or dried mushroom blend can be calculated based on the subject's body weight. In some embodiments, the composition comprises about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, or about 0.2 mg/kg of the subject's body weight. In some embodiments the psilocybin or a derivative thereof, or psilocin or a derivative thereof is in the form of a dried mushroom or dried mushroom blend, wherein the composition comprises about 0.01 mg/kg to about 0.2 mg/kg, about 0.02 mg/kg to about 0.2 mg/kg, about 0.03 mg/kg to about 0.2 mg/kg, about 0.04 mg/kg to about 0.2 mg/kg, about 0.05 mg/kg to about 0.2 mg/kg, about 0.06 mg/kg to about 0.2 mg/kg, about 0.07 mg/kg to about 0.2 mg/kg, about 0.08 mg/kg to about 0.2 mg/kg, about 0.09 mg/kg to about 0.2 mg/kg, about 0.07 mg/kg to about 0.1 mg/kg, about 0.06 mg/kg to about 0.11 mg/kg, about 0.05 mg/kg to about 0.13 mg/kg, about 0.4 mg/kg to about 0.13 mg/kg, about 0.4 mg/kg to about 0.14 mg/kg, about 0.4 mg/kg to about 0.14 mg/kg of the subject's body weight.

The composition may include an amount of synthetic psilocybin or a derivative thereof that provides from about 0.01 mg/day up to about 10 mg/day of the psilocybin or the derivative thereof. This includes about 0.01 mg/day, about 0.02 mg/day, about 0.03 mg/day, about 0.04 mg/day, about 0.05 mg/day, about 0.06 mg/day, about 0.07 mg/day, about 0.08 mg/day, about 0.09 mg/day, about 0.1 mg/day, about 0.11 mg/day, about 0.12 mg/day, about 0.13 mg/day, about 0.14 mg/day, about 0.15 mg/day, about 0.16 mg/day, about 0.17 mg/day, about 0.18 mg/day, about 0.19 mg/day, about 0.2 mg/day, about 0.21 mg/day, about 0.22 mg/day, about 0.23 mg/day, about 0.24 mg/day, about 0.25 mg/day, about 0.26 mg/day, about 0.27 mg/day, about 0.28 mg/day, about 0.29 mg/day, about 0.3 mg/day, about 0.31 mg/day, about 0.32 mg/day, about 0.33 mg/day, about 0.34 mg/day, about 0.35 mg/day, about 0.36 mg/day, about 0.37 mg/day, about 0.38 mg/day, about 0.39 mg/day, about 0.4 mg/day, about 0.41 mg/day, about 0.42 mg/day, about 0.43 mg/day, about 0.44 mg/day, about 0.45 mg/day, about 0.46 mg/day, about 0.47 mg/day, about 0.48 mg/day, about 0.49 mg/day, about 0.5 mg/day, about 0.51 mg/day, about 0.52 mg/day, about 0.53 mg/day, about 0.54 mg/day, about 0.55 mg/day, about 0.56 mg/day, about 0.57 mg/day, about 0.58 mg/day, about 0.59 mg/day, about 0.6 mg/day, about 0.7 mg/day, about 0.8 mg/day, about 0.9 mg/day, about 1 mg/day, about 2 mg/day, about 3 mg/day, about 4 mg/day, about 5 mg/day, about 6 mg/day, about 7 mg/day, about 8 mg/day, about 9 mg/day, or about 10 mg/day, and any amounts therebetween. For example, the dosage form may include an amount of synthetic psilocybin that provides from about 0.01 mg/day up to about 6 mg/day psilocybin. Other non-limiting ranges include about 0.1 mg/day up to about 9 mg/day, about 0.1 mg/day up to about 8 mg/day, about 0.1 mg/day up to about 7 mg/day, about 0.1 mg/day up to about 6 mg/day, about 0.1 mg/day up to about 5 mg/day, about 0.1 mg/day up to about 4 mg/day, about 0.1 mg/day up to about 3 mg/day, about 0.1 mg/day up to about 2 mg/day, about 0.1 mg/day up to about 1 mg/day, about 0.1 mg/day up to about 0.9 mg/day, about 0.1 mg/day up to about 0.8 mg/day, about 0.1 mg/day up to about 0.7 mg/day, about 0.1 mg/day up to about 0.6 mg/day, about 0.1 mg/day up to about 0.5 mg/day, about 0.1 mg/day up to about 0.4 mg/day, 0.2 mg/day up to about 9 mg/day, about 0.2 mg/day up to about 8 mg/day, about 0.2 mg/day up to about 7 mg/day, about 0.2 mg/day up to about 6 mg/day, about 0.2 mg/day up to about 5 mg/day, about 0.2 mg/day up to about 4 mg/day, about 0.2 mg/day up to about 3 mg/day, about 0.2 mg/day up to about 2 mg/day, about 0.2 mg/day up to about 1 mg/day, about 0.2 mg/day up to about 0.9 mg/day, about 0.2 mg/day up to about 0.8 mg/day, about 0.2 mg/day up to about 0.7 mg/day, about 0.2 mg/day up to about 0.6 mg/day, about 0.2 mg/day up to about 0.5 mg/day, about 0.2 mg/day up to about 0.4 mg/day, about 0.2 mg/day up to about 0.3 mg/day, about 0.25 mg/day up to about 0.4 mg/day, about 0.3 mg/day up to about 0.4 mg/day, about 0.5 mg/day up to about 9 mg/day, about 0.5 mg/day up to about 8 mg/day, about 0.5 mg/day up to about 7 mg/day, about 0.5 mg/day up to about 6 mg/day, about 0.5 mg/day up to about 5 mg/day, about 0.5 mg/day up to about 4 mg/day, about 0.5 mg/day up to about 3 mg/day, about 0.5 mg/day up to about 2 mg/day, about 0.5 mg/day up to about 1 mg/day, about 1 mg/day up to about 9 mg/day, about 1 mg/day up to about 8 mg/day, about 1 mg/day up to about 7 mg/day, about 1 mg/day up to about 6 mg/day, about 1 mg/day up to about 5 mg/day, 1 mg/day up to about 4 mg/day, about 1 mg/day up to about 3 mg/day, about 1 mg/day up to about 2 mg/day of psilocybin.

In some embodiments, the synthetic psilocybin or derivative thereof or psilocin or derivative thereof, can be calculated based on the subject's body weight. In some embodiments, the composition comprises about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, or about 0.1 mg/kg of the subject's body weight. In some embodiments, the synthetic psilocybin or a derivative thereof, or psilocin or a derivative thereof can comprise about 0.01 mg/kg to about 0.1 mg/kg, about 0.02 mg/kg to about 0.1 mg/kg, about 0.03 mg/kg to about 0.1 mg/kg, about 0.04 mg/kg to about 0.1 mg/kg, about 0.01 mg/kg to about 0.09 mg/kg, about 0.01 mg/kg to about 0.08 mg/kg, about 0.01 mg/kg to about 0.07 mg/kg, about 0.01 mg/kg to about 0.06 mg/kg, about 0.01 mg/kg to about 0.05 mg/kg, about 0.01 mg/kg to about 0.04 mg/kg, about 0.02 mg/kg to about 0.08 mg/kg, about 0.03 mg/kg to about 0.07 mg/kg, about 0.03 mg/kg to about 0.06 mg/kg, about 0.03 mg/kg to about 0.05 mg/kg, about 0.03 mg/kg to about 0.045 mg/kg, or about 0.03 mg/kg to about 0.04 mg/kg of the subject's body weight.

The composition may include psilocybin or a derivative thereof in an amount of from about 0.05 g to about 0.6 g. In some embodiments, the psilocybin or derivative thereof, or the psilocin or derivative thereof is in the form of a dried mushroom, such as Psilocybe cubensis, or dried mushroom blend. In some embodiments, the psilocybin is an extract from natural dried mushrooms. This includes about 0.05 g, about 0.06 g, about 0.07 g, about 0.08 g, about 0.09 g, about 0.1 g, about 0.12 g, about 0.14 g, about 0.16 g, about 0.18 g, about 0.2 g, about 0.22 g, about 0.24 g, about 0.26 g, about 0.28 g, about 0.30 g, about 0.32 g, about 0.34 g, about 0.36 g, about 0.38 g, about 0.4 g, about 0.42 g, about 0.44 g, about 0.46 g, about 0.48 g, about 0.5 g, about 0.52 g, about 0.54 g, about 0.56 g, about 0.58 g, about 0.6 g. The compositions may include psilocybin or a derivative thereof in an amount from about 0.05 g mg to about 0.6 g, about 0.1 g to about 0.6 g, about 0.1 g to about 0.5 g, about 0.1 g to about 0.4 g, about 0.1 g to about 0.3 g, about 0.1 g to about 0.2 g, about 0.2 g to about 0.6 g, about 0.2 g to about 0.5 g, about 0.2 g to about 0.4 g, about 0.2 g to about 0.3 g, about 0.3 g to about 0.6 g, about 0.3 g to about 0.5 g, about 0.3 g to about 0.4 g, about 0.4 g to about 0.6 g, about 0.4 g to about 0.5 g. In some embodiments, the amount of psilocybin or derivative thereof or psilocin or derivative thereof is about 0.25 g to about 0.5 g of dried mushrooms or a dried mushroom blend.

The composition may include psilocybin or a derivative thereof, or psilocin or a derivative thereof is in the form of a dried mushroom, such as Psilocybe cubensis, or extract from a dried mushroom blend, wherein the composition comprises about 0.001 mg/kg, about 0.002 mg/kg, about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.011 mg/kg, about 0.012 mg/kg, about 0.013 mg/kg, about 0.014 mg/kg, about 0.015 mg/kg, about 0.016 mg/kg, about 0.017 mg/kg, about 0.018 mg/kg, about 0.019 mg/kg, or about 0.02 mg/kg of the subject's body weight. In some embodiments the psilocybin or a derivative thereof, or psilocin or a derivative thereof is in the form of a dried mushroom or dried mushroom blend, wherein the composition comprises about 0.001 mg/kg to about 0.02 mg/kg, about 0.002 mg/kg to about 0.02 mg/kg, about 0.003 mg/kg to about 0.02 mg/kg, about 0.004 mg/kg to about 0.02 mg/kg, about 0.005 mg/kg to about 0.02 mg/kg, about 0.006 mg/kg to about 0.02 mg/kg, about 0.007 mg/kg to about 0.02 mg/kg, about 0.008 mg/kg to about 0.02 mg/kg, about 0.009 mg/kg to about 0.02 mg/kg, about 0.007 mg/kg to about 0.01 mg/kg, about 0.006 mg/kg to about 0.011 mg/kg, about 0.005 mg/kg to about 0.013 mg/kg, about 0.04 mg/kg to about 0.013 mg/kg, about 0.04 mg/kg to about 0.014 mg/kg, about 0.04 mg/kg to about 0.014 mg/kg of the subject's body weight.

As noted above, the psilocybin may be isolated, extracted, purified, crystalline, amorphous, or deuterated psilocybin. In some embodiments, the psilocybin is purified psilocybin manufactured under Good Manufacturing Practices (GMP) conditions. Alternatively, as also noted above, the psilocybin may be in the form of dried Psilocybe cubensis, as discussed in more detail above in the context of mushroom blends.

### Cannabinoids and Derivatives

The cannabidiol may be synthetic cannabidiol or may be purified cannabidiol that has been extracted from a natural source. In some embodiments, the cannabidiol can be full synthetic cannabidiol, nano-emulsified cannabidiol, deuterated cannabidiol, naturally extracted cannabidiol, or amorphous cannabidiol. In some embodiments, the cannabinoid can be a nanoparticulate cannabinoid, such as nanoparticulate cannabidiol. In some embodiments, the nanoparticulate cannabinoid increases absorption of the cannabinoid. In some embodiments, the absorption is increased between 5% and 50% better than non-nanoparticulate formulations of the cannabinoid. In some embodiments, the absorption is increased between 10% and 50%, between 15% and 50%, between 20% and 40%, between 20% and 35%, between 25% and 30% better than non-nanoparticulate formulations of the cannabinoid. In some embodiments, the absorption is increased about 10% more, about 15% more, about 20% more, about 25% more, about 30% more, about 35% more, about 40% more, about 45% more, about 50% more than non-nanoparticulate formulations of the cannabinoid. In some embodiments, additional synthetic cannabinoids such as CBG or CBA and/or synthetic terpenes can be added to confer additional functionality.

Various methods are available in the art for obtaining an extract from a natural source (e.g., a cannabidiol-containing extract). For example, an extract can be prepared by contacting plant material with supercritical or subcritical carbon dioxide. In some embodiments, an extract can be prepared by contacting plant material with a heated gas (e.g., at a temperature in excess of 100° C.) sufficient to volatilize one or more components of the plant material to be extracted and condensing the vapor to form an extract.

As set forth above, the composition of the methods and compositions described herein can include a cannabinoid or a derivative thereof. The composition can include the cannabinoid in combination with psilocybin or a derivative thereof. In some embodiments, the composition comprises a cannabinoid or a derivative thereof in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt % 30 wt %, 35 wt %, 40 wt % 45 wt % 50 wt %, 55 wt % 60 wt %, 65 wt %, 70 wt %, 75 wt %, or 80 wt %. In some embodiments, the composition comprises a cannabinoid or a derivative thereof in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt % 5 wt %, 6 wt %, 7 wt %, 8 wt %, 9 wt % 10 wt % 15 wt % 20 wt % 25 wt % 30 wt % 35 wt % 40 wt %, 45 wt %, 50 wt %, 55 wt %, 60 wt %, 65 wt %, 70 wt %, 75 wt %, or 80 wt %. In some embodiments, the cannabinoid is cannabidiol.

The compositions may include from about 1 mg to about 1000 mg of a cannabinoid. The cannabinoid in various embodiments is cannabidiol (CBD). In some embodiments, the cannabinoid is synthetic CBD. The composition may include, e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, or about 1000 mg cannabinoid (e.g., CBD), or any amount therebetween.

Thus, the compositions may include a synthetic cannabinoid in an amount from about 1 mg to about 1000 mg, about 1 mg to about 600 mg, about 1 mg to about 500 mg, about 1 mg to about 400 mg, about 1 mg to about 300 mg, about 1 mg to about 200 mg, about 1 mg to about 100 mg, about 1 mg to about 50 mg, about 10 mg to about 1000 mg, about 10 mg to about 500 mg, about 10 mg to about 400 mg, about 10 mg to about 300 mg, about 10 mg to about 200 mg, about 10 mg to about 100 mg, about 100 mg to about 1000 mg, about 100 mg to about 900 mg, about 100 mg to about 800 mg, about 100 mg to about 700 mg, about 100 mg to about 600 mg, about 200 mg to about 1000 mg, about 200 mg to about 900 mg, about 200 mg to about 800 mg, about 200 mg to about 700 mg, about 200 mg to about 600 mg, about 250 mg to about 1000 mg, about 250 mg to about 900 mg, about 250 mg to about 800 mg, about 250 mg to about 700 mg, about 250 mg to about 600 mg, about 300 mg to about 1000 mg, about 300 mg to about 900 mg, about 300 mg to about 800 mg, about 300 mg to about 700 mg, about 300 mg to about 600 mg, about 350 mg to about 900 mg, about 350 mg to about 800 mg, about 350 mg to about 700 mg, about 350 mg to about 650 mg, about 350 mg to about 600 mg. In some embodiments, the compositions may include a synthetic cannabinoid in an amount from about 1 mg to about 5 mg, about 1 mg to about 10 mg, about 1 mg to about 15 mg, about 1 mg to about 20 mg, about 1 mg to about 25 mg, about 1 mg to about 30 mg, about 1 mg to about 35 mg, about 1 mg to about 40 mg, about 1 mg to about 45 mg, about 1 mg to about 50 mg, about 1 mg to about 55 mg, about 1 mg to about 60 mg, about 1 mg to about 65 mg, about 1 mg to about 70 mg, about 1 mg to about 75 mg, about 1 mg to about 80 mg, about 1 mg to about 85 mg, about 1 mg to about 90 mg, about 1 mg to about 95 mg, about 1 mg to about 100 mg, about 1 mg to about 105 mg, about 1 mg to about 110 mg, about 1 mg to about 115 mg, about 1 mg to about 120 mg, about 1 mg to about 125 mg, about 1 mg to about 130 mg, about 1 mg to about 135 mg, about 1 mg to about 140 mg, about 1 mg to about 145 mg, about 1 mg to about 150 mg, about 1 mg to about 155 mg, about 1 mg to about 160 mg, about 1 mg to about 165 mg, about 1 mg to about 170 mg, about 1 mg to about 175 mg, about 1 mg to about 180 mg, about 1 mg to about 185 mg, about 1 mg to about 190 mg, about 1 mg to about 195 mg, about 1 mg to about 200 mg, about 1 mg to about 205 mg, about 1 mg to about 210 mg, about 1 mg to about 215 mg, about 1 mg to about 220 mg, about 1 mg to about 225 mg, about 1 mg to about 230 mg, about 1 mg to about 235 mg, about 1 mg to about 240 mg, about 1 mg to about 245 mg, about 1 mg to about 250 mg, about 1 mg to about 255 mg, about 1 mg to about 260 mg, about 1 mg to about 265 mg, about 1 mg to about 270 mg, about 1 mg to about 275 mg, about 1 mg to about 280 mg, about 1 mg to about 285 mg, about 1 mg to about 290 mg, about 1 mg to about 295 mg, or about 1 mg to about 300 mg.

In some embodiments, the synthetic cannabidiol or derivative thereof, can be calculated based on the subject's body weight. In some embodiments, the composition comprises about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, or about 15 mg/kg of the subject's body weight. In some embodiments, the synthetic cannabidiol or a derivative thereof can comprise about 1 mg/kg to about 15 mg/kg, about 1 mg/kg to about 13 mg/kg, about 1 mg/kg to about 11 mg/kg, about 2 mg/kg to about 11 mg/kg, about 3 mg/kg to about 10 mg/kg, about 4 mg/kg to about 9 mg/kg of the subject's body weight.

In some embodiments, the cannabidiol or derivative thereof can be a natural cannabidiol, for example in the form of a full kief. In some embodiments, the composition comprises about 1 to about 1000 mg of natural cannabidiol, for example, about 1 mg, about 5, about 10 mg, about 15 mg, about 20 mg, about 30 mg, about 35 mg, about 40 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 125 mg, about 126 mg, about 127 mg, about 128 mg, about 129, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 155 mg, about 160 mg, about 165 mg, about 170 mg, about 175 mg, about 180 mg, about 185 mg, about 190 mg, about 195 mg, about 200 mg cannabinoid (e.g., CBD), or any amount therebetween. In some embodiments, the composition comprises natural CBD in an amount from about 1-1000 mg, e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, or about 1000 mg cannabinoid (e.g., CBD), or any amount therebetween. In some embodiments, the composition comprises natural CBD in an amount from about 1 mg to about 1000 mg, about 1 mg to about 600 mg, about 1 mg to about 500 mg, about 1 mg to about 400 mg, about 1 mg to about 300 mg, about 1 mg to about 200 mg, about 1 mg to about 100 mg, about 1 mg to about 50 mg, about 10 mg to about 1000 mg, about 10 mg to about 500 mg, about 10 mg to about 400 mg, about 10 mg to about 300 mg, about 10 mg to about 200 mg, about 10 mg to about 100 mg, about 100 mg to about 1000 mg, about 100 mg to about 900 mg, about 100 mg to about 800 mg, about 100 mg to about 700 mg, about 100 mg to about 600 mg, about 200 mg to about 1000 mg, about 200 mg to about 900 mg, about 200 mg to about 800 mg, about 200 mg to about 700 mg, about 200 mg to about 600 mg, about 250 mg to about 1000 mg, about 250 mg to about 900 mg, about 250 mg to about 800 mg, about 250 mg to about 700 mg, about 250 mg to about 600 mg, about 300 mg to about 1000 mg, about 300 mg to about 900 mg, about 300 mg to about 800 mg, about 300 mg to about 700 mg, about 300 mg to about 600 mg, about 350 mg to about 900 mg, about 350 mg to about 800 mg, about 350 mg to about 700 mg, about 350 mg to about 650 mg, about 350 mg to about 600 mg. In some embodiments, the compositions may include a natural cannabinoid (e.g., in the form of a full kief) in an amount from about 1 mg to about 5 mg, about 1 mg to about 10 mg, about 1 mg to about 15 mg, about 1 mg to about 20 mg, about 1 mg to about 25 mg, about 1 mg to about 30 mg, about 1 mg to about 35 mg, about 1 mg to about 40 mg, about 1 mg to about 45 mg, about 1 mg to about 50 mg, about 1 mg to about 55 mg, about 1 mg to about 60 mg, about 1 mg to about 65 mg, about 1 mg to about 70 mg, about 1 mg to about 75 mg, about 1 mg to about 80 mg, about 1 mg to about 85 mg, about 1 mg to about 90 mg, about 1 mg to about 95 mg, about 1 mg to about 100 mg, about 1 mg to about 105 mg, about 1 mg to about 110 mg, about 1 mg to about 115 mg, about 1 mg to about 120 mg, about 1 mg to about 125 mg, about 1 mg to about 130 mg, about 1 mg to about 135 mg, about 1 mg to about 140 mg, about 1 mg to about 145 mg, about 1 mg to about 150 mg, about 1 mg to about 155 mg, about 1 mg to about 160 mg, about 1 mg to about 165 mg, about 1 mg to about 170 mg, about 1 mg to about 175 mg, about 1 mg to about 180 mg, about 1 mg to about 185 mg, about 1 mg to about 190 mg, about 1 mg to about 195 mg, about 1 mg to about 200 mg, about 1 mg to about 205 mg, about 1 mg to about 210 mg, about 1 mg to about 215 mg, about 1 mg to about 220 mg, about 1 mg to about 225 mg, about 1 mg to about 230 mg, about 1 mg to about 235 mg, about 1 mg to about 240 mg, about 1 mg to about 245 mg, about 1 mg to about 250 mg, about 1 mg to about 255 mg, about 1 mg to about 260 mg, about 1 mg to about 265 mg, about 1 mg to about 270 mg, about 1 mg to about 275 mg, about 1 mg to about 280 mg, about 1 mg to about 285 mg, about 1 mg to about 290 mg, about 1 mg to about 295 mg, or about 1 mg to about 300 mg.

In some embodiments, the natural cannabidiol, can be calculated based on the subject's body weight. In some embodiments, the composition comprises about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, or about 3.0 mg/kg of the subject's body weight. In some embodiments, the natural cannabidiol can comprise about 0.1 mg/kg to about 3 mg/kg, about 0.2 mg/kg to about 3 mg/kg, about 0.3 mg/kg to about 3 mg/kg, about 0.4 mg/kg to about 3 mg/kg, about 0.5 mg/kg to about 3 mg/kg, about 0.6 mg/kg to about 3 mg/kg, about 0.7 mg/kg to about 3 mg/kg, about 0.5 mg/kg to about 2.9 mg/kg, about 0.5 mg/kg to about 2.8 mg/kg, about 0.5 mg/kg to about 2.7 mg/kg, about 0.5 mg/kg to about 2.6 mg/kg, about 0.5 mg/kg to about 2.5 mg/kg, about 0.7 mg/kg to about 2.9 mg/kg, about 0.7 mg/kg to about 2.8 mg/kg, about 0.7 mg/kg to about 2.7 mg/kg, about 0.7 mg/kg to about 2.6 mg/kg, or about 0.7 mg/kg to about 2.5 mg/kg of the subject's body weight.

### Mushroom Blends

In various embodiments, the methods and compositions described herein involve or contain mushroom blends. In some embodiments, the mushroom blends are dried mushroom blends. Typically, the dried mushroom blends are prepared by a process that includes steam activation prior to drying, which may be carried out by methods known in the art. In some embodiments, the mushroom blends may be formulated as mushroom extracts (e.g., liquid).

Each of the mushrooms referenced herein are available commercially, including in dried form (e.g., steam-activated dried form). Mushrooms may be grown under organic practices and steam activated after harvest to break down parts of the mushroom fruiting body and/or mycelium, before being freeze-dried to powder. Thus, the compositions and methods described herein may be prepared and practiced with specific combinations of specific amounts of commercially available mushroom products.

As set forth above, the mushroom blends comprise two or more selected from the group consisting of Inonotus obliquus (also known as the Chaga mushroom), Agaricus subrufescens (also known as Agaricus blazei or the almond mushroom), Laricifomes officinalis (also known as the Agarikon mushroom), Trametes versicolor (also known as Turkey Tail), Ganoderma lingzhi (also known as the reishi mushroom), Schizophyllum commune (also known as the suehirotake mushroom or split-fold mushroom), Hericium erinaceus (also known as the Lion's Mane mushroom), Wolfiporia extensa (also known as the poria mushroom), Cordyceps sp. (such as, but not limited to, Cordyceps militaris), Fomes fomentarius (also known as the True Tinder Polypore), Pleurotus ostreatus (also known as the oyster mushroom), Phellinus linteus (also known as the Mesima mushroom), Grifola frondosa (also known as the maitake mushroom), and Lentinula edodes (also known as shiitake mushroom), various sub-combinations thereof, and, optionally, Psilocybe cubensis.

Thus, the mushroom blends may comprise Inonotus obliquus, Agaricus subrufescens, Laricifomes officinalis, Trametes versicolor, Ganoderma lingzhi, Schizophyllum commune, Hericium erinaceus, Wolfiporia extensa, Cordyceps sp., Fomes fomentarius, Pleurotus ostreatus, Phellinus linteus, Grifola frondosa, and Lentinula edodes, or, in embodiments comprising Psilocybe cubensis, may comprise, consist essentially of, or consist of Inonotus obliquus, Agaricus subrufescens, Laricifomes officinalis, Trametes versicolor, Ganoderma lingzhi, Schizophyllum commune, Hericium erinaceus, Wolfiporia extensa, Cordyceps sp., Fomes fomentarius, Pleurotus ostreatus, Phellinus linteus, Grifola frondosa, and Lentinula edodes, or various sub-combinations thereof. In some embodiments, the mushroom blends further comprise dried Psilocybe cubensis.

The mushroom blend may comprise Inonotus obliquus (also known as the Chaga mushroom), Laricifomes officinalis (also known as the Agarikon mushroom), Trametes versicolor (also known as Turkey Tail), Ganoderma lingzhi (also known as the reishi mushroom), Hericium erinaceus (also known as the Lion's Mane mushroom), Cordyceps sp. (such as, but not limited to, Cordyceps militaris). The mushroom blend may consist essentially of or consist of Inonotus obliquus, Laricifomes officinalis, Trametes versicolor, Ganoderma lingzhi, Hericium erinaceus, and Cordyceps sp.

The mushroom blend may comprise Hericium erinaceus (also known as the Lion's Mane mushroom) and Ganoderma lingzhi (also known as the reishi mushroom). The mushroom blend may consist essentially of or consist of Ganoderma lingzhi (also known as the reishi mushroom), Hericium erinaceus (also known as the Lion's Mane mushroom).

The dried Inonotus obliquus may be present in the composition in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt, 9 wt % 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Inonotus obliquus in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt % 6 wt %, 7 wt % 8 wt %, 9 wt %, 10 wt %, 15 wt %, 20 wt %, 25 wt %, 30 wt % 35 wt %, 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Inonotus obliquus. In some embodiments, the composition comprises between about 0.01 and about 4.5 mg, about 0.01 g and about 4 g, about 0.01 g and about 3.5 g, about 0.01 mg and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Inonotus obliquus. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 g to about 0.5 g, about 0.3 g to about 0.5 g Inonotus obliquus.

The dried Agaricus subrufescens may be present in the composition in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Agaricus subrufescens in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt % 8 wt % 9 wt % 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Agaricus subrufescens. In some embodiments, the composition comprises between about 0.01 g and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Agaricus subrufescens. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Agaricus subrufescens.

Dried Laricifomes officinalis may be present in the composition in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt, 9 wt % 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Laricifomes officinalis in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt % 5 wt % 6 wt %, 7 wt %, 8 wt %, 9 wt % 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Laricifomes officinalis. In some embodiments, the composition comprises between about 0.01 g and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Laricifomes officinalis. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Laricifomes officinalis.

Dried Trametes versicolor may be present in the composition in an amount of about or at least about 00.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt, 9 wt % 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Trametes versicolor in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt %, 9 wt % 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Trametes versicolor. In some embodiments, the composition comprises between about 0.01 g and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Trametes versicolor. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Trametes versicolor.

Dried Ganoderma lingzhi may be present in the composition in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt % 4 wt % 5 wt % 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Ganoderma lingzhi in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt % 4 wt %, 5 wt %, 6 wt %, 7 wt %, 8 wt %, 9 wt % 10 wt %, 15 wt %, 20 wt, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Ganoderma lingzhi. In some embodiments, the composition comprises between about 0.01 g and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Ganoderma lingzhi. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Ganoderma lingzhi.

Dried Schizophyllum commune may be present in the composition in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, wt %, 2 wt %, 3 wt %, 4 wt %, 5 wt %, 6 wt %, 7 wt %, 8 wt %, 9 wt % 10 wt %, 15 wt % 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Schizophyllum commune in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt % 5 wt % 6 wt %, 7 wt %, 8 wt %, 9 wt % 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Schizophyllum commune. In some embodiments, the composition comprises between about 0.01 g and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Schizophyllum commune. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Schizophyllum commune.

Dried Hericium erinaceus may be present in the composition in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt % 4 wt % 5 wt % 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Hericium erinaceus in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt % 4 wt %, 5 wt %, 6 wt %, 7 wt %, 8 wt %, 9 wt % 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Hericium erinaceus. In some embodiments, the composition comprises between about 0.01 and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Hericium erinaceus. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Hericium erinaceus.

Dried Wolfiporia extensa may be present in the composition in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt % 4 wt % 5 wt % 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Wolfiporia extensa in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt % 5 wt % 6 wt %, 7 wt %, 8 wt %, 9 wt % 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Wolfiporia extensa. In some embodiments, the composition comprises between about 0.01 g and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Wolfiporia extensa. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Wolfiporia extensa.

Dried Cordyceps sp. may be present in the composition in an amount of about or at least about 0.01 wt %, 0.02 wt %, 0.03 wt %, 0.04 wt %, 0.05 wt %, 0.1 wt %, 0.2 wt %, 0.3 wt %, 0.4 wt % 0.5 wt %, 0.75 wt %, 1 wt %, 2 wt %, 3 wt % 4 wt % 5 wt % 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Cordyceps sp. in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt % 8 wt %, 9 wt %, 10 wt %, 15 wt %, 20 wt %, 25 wt %, 30 wt %, 35 wt % 40 wt %, 45 wt %, 50 wt %, 55 wt %, 60 wt %, 65 wt %, 70 wt %, 75 wt %, or 80 wt %. In some embodiments, the dried Cordyceps sp. present in the composition comprises dried Cordyceps militaris. In some embodiments, the composition comprises between about 0.01 g and about 5 g Cordyceps sp. In some embodiments, the composition comprises between about 0.01 g and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Cordyceps sp. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Cordyceps sp.

Dried Fomes fomentarius may be present in the composition in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Fomes fomentarius in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt % 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Fomes fomentarius. In some embodiments, the composition comprises between about 0.01 g and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Fomes fomentarius. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Fomes fomentarius.

Dried Pleurotus ostreatus may be present in the composition in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Pleurotus ostreatus in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt % 4 wt %, 5 wt %, 6 wt %, 7 wt %, 8 wt %, 9 wt % 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Pleurotus ostreatus. In some embodiments, the composition comprises between about 0.01 g and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Pleurotus ostreatus. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Pleurotus ostreatus.

Dried Phellinus linteus may be present in the composition in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Phellinus linteus in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt % 5 wt %, 6 wt % 7 wt % 8 wt %, 9 wt % 10 wt %, 15 wt %, 20 wt % 25 wt % 30 wt % 35 wt % 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Phellinus linteus. In some embodiments, the composition comprises between about 0.01 g and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, 0.01 g and about 1 g about 0.01 g and about 0.5 g, about 0.01 g and about 0.4 g, about 0.01 g and about 0.3 g, about 0.01 g and about 0.2 g, and about 0.01 and about 0.1 g Phellinus linteus. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Phellinus linteus.

Dried Grifola frondosa may be present in the composition in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Grifola frondosa in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt % 7 wt % 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Grifola frondosa. In some embodiments, the composition comprises between about 0.01 g and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Grifola frondosa. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Grifola frondosa.

Dried Lentinula edodes may be present in the composition in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Lentinula edodes in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt % 5 wt %, 6 wt % 7 wt % 8 wt %, 9 wt %, 10 wt %, 15 wt %, 20 wt %, 25 wt % 30 wt %, 35 wt % 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Lentinula edodes. In some embodiments, the composition comprises between about 0.01 g and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Lentinula edodes. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Lentinula edodes.

In some embodiments, the mushroom blends further comprise a dried mushroom(s) of the genus Psilocybe. In some embodiments, the dried mushroom is Psilocybe cubensis. When present, dried Psilocybe may be present in an amount of about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt % 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises Psilocybe in an amount of no more than about 0.01 wt %, 0.02 wt %, 0.03 wt %, 0.04 wt %, 0.05 wt %, 0.1 wt %, 0.2 wt %, 0.3 wt % 0.4 wt % 0.5 wt %, 0.75 wt % wt %, 2 wt %, 3 wt %, 4 wt %, 5 wt % 6 wt % 7 wt % 8 wt %, 9 wt % 10 wt %, 15 wt % 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %. In some embodiments, the composition comprises between about 0.01 g and about 5 g Psilocybe cubensis. In some embodiments, the composition comprises between about 0.01 g and about 4.5 g, about 0.01 g and about 4 g, about 0.01 g and about 3.5 g, about 0.01 g and about 3 g, about 0.01 g and about 2.5 g, about 0.01 g and about 2 g, about 0.01 g and about 1.5 g, about 0.01 g and about 1 g, about 0.01 g and about 0.5 g, about 0.01 g and about 0.4 g, about 0.01 g and about 0.3 g, about 0.01 g and about 0.2 g, and about 0.01 and about 0.1 g Psilocybe cubensis. In some embodiments, the composition comprises between about 0.02 and about 0.5 g, about 0.03 and about 0.5 g, about 0.04 and about 0.5 g, about 0.05 and about 0.5 g, about 0.06 and about 0.5 g, about 0.07 and about 0.5 g, about 0.08 and about 0.5 g, about 0.09 and about 0.5 g, about 0.1 g to about 0.5 g, about 0.15 g to about 0.5 g, about 0.2 g to about 0.5 g, about 0.25 to about 0.5, about 0.3 g to about 0.5 g Psilocybe cubensis. In some embodiments, the Psilocybe is a part of a mushroom blend comprising two or more mushrooms.

In some embodiments, as described elsewhere in this application, the Psilocybe is administered/taken as a part of a loading dose and/or a microdose (maintenance dose) separately from the mushroom blend. Dosage amounts for Psilocybe when taken as a loading or microdose (not as part of a mushroom blend) are described elsewhere in the disclosure.

The dried Inonotus obliquus may be present in the compositions in an amount from about 100 mg to about 400 mg by dry weight. This includes an amount from about 100 mg to about 200 mg, about 100 mg to about 300 mg, about 200 mg to about 300 mg, about 210 mg to about 290 mg, about 220 mg to about 280 mg, about 230 mg to about 275 mg, about 250 to about 275 mg, about 260 mg to about 270 mg, about 200 mg to about 400 mg, or about 300 mg to about 400 mg by dry weight. For example, dried Inonotus obliquus may be present in the compositions in an amount of about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 mg, or any amount therebetween, by dry weight. In some embodiments, dried Inonotus obliquus is present in the compositions in an amount of about 268.9 mg by dry weight.

The dried Agaricus subrufescens may be present in the compositions in an amount from about 10 mg to about 50 mg by dry weight. This includes an amount from about 10 mg to about 20 mg, about 10 mg to about 30 mg, about 10 mg to about 40 mg, about 20 mg to about 30 mg, about 20 mg to about 40 mg, about 20 mg to about 50 mg, about 30 mg to about 40 mg, about 30 mg to about 50 mg, about 20 mg to about 25 mg, about 21 mg to about 24 mg, about 21 mg to about 23 mg, about 21 mg to about 22 mg, or about 40 mg to about 50 mg by dry weight. Thus, dried Agaricus subrufescens may be present in the compositions in an amount of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg, or any amount therebetween, by dry weight. In some embodiments, dried Agaricus subrufescens is present in the compositions in an amount of about 20.4 mg by dry weight.

Dried Laricifomes officinalis may be present in the compositions in an amount from about 100 mg to about 400 mg by dry weight. This includes an amount from about 100 mg to about 200 mg, about 100 mg to about 300 mg, about 200 mg to about 300 mg, about 210 mg to about 290 mg, about 220 mg to about 280 mg, about 230 mg to about 270 mg, about 240 mg to about 260 mg, about 245 mg about 255 mg, about 247 mg to about 253 mg, about 200 mg to about 400 mg, or about 300 mg to about 400 mg by dry weight. Thus, dried Laricifomes officinalis may be present in the compositions in an amount of about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 mg, or any amount therebetween, by dry weight. In some embodiments, dried Laricifomes officinalis is present in the compositions in an amount of about 251.9 mg by dry weight.

Dried Trametes versicolor may be present in the compositions in an amount from about 100 mg to about 400 mg by dry weight. This includes an amount from about 100 mg to about 200 mg, about 100 mg to about 300 mg, about 200 mg to about 300 mg, about 210 mg to about 290 mg, about 220 mg to about 280 mg, about 230 mg to about 270 mg, about 240 mg to about 260 mg, about 245 mg about 260 mg, about 250 mg to about 260 mg, about 253 mg to about 257 mg, about 200 mg to about 400 mg, or about 300 mg to about 400 mg by dry weight. Thus, dried Trametes versicolor may be present in the compositions in an amount of about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 mg, or any amount therebetween, by dry weight. In some embodiments, dried Trametes versicolor is present in the compositions in an amount of about 255.3 mg by dry weight.

Dried Ganoderma lingzhi may be present in the compositions in an amount from about 100 mg to about 400 mg by dry weight. This includes an amount from about 100 mg to about 200 mg, about 100 mg to about 300 mg, about 200 mg to about 300 mg, about 210 mg to about 290 mg, about 220 mg to about 280 mg, about 230 mg to about 270 mg, about 240 mg to about 270 mg, about 250 mg about 270 mg, about 260 mg to about 270 mg, about 200 mg to about 400 mg, or about 300 mg to about 400 mg by dry weight. Thus, dried Ganoderma lingzhi may be present in the compositions in an amount of about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 mg, or any amount therebetween, by dry weight. In some embodiments, dried Ganoderma lingzhi is present in the compositions in an amount of about 268.9 mg by dry weight.

Dried Schizophyllum commune may be present in the compositions in an amount from about 10 mg to about 50 mg by dry weight. This includes an amount from about 10 mg to about 20 mg, about 11 mg to about 20 mg, about 12 mg to about 20 mg, about 13 mg to about 20 mg, about 14 mg to about 20 mg, about 15 mg to about 20 mg, about 16 mg to about 20 mg, about 16 mg to about 19 mg, about 16 mg to about 18 mg, about 10 mg to about 30 mg, about 10 mg to about 40 mg, about 20 mg to about 30 mg, about 20 mg to about 40 mg, about 20 mg to about 50 mg, about 30 mg to about 40 mg, about 30 mg to about 50 mg, or about 40 mg to about 50 mg by dry weight. Thus, dried Schizophyllum commune may be present in the compositions in an amount of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg, or any amount therebetween, by dry weight. In some embodiments, dried Schizophyllum commune is present in the compositions in an amount of about 17 mg by dry weight.

Dried Hericium erinaceus may be present in the compositions in an amount from about 100 mg to about 400 mg by dry weight. This includes an amount from about 100 mg to about 200 mg, about 100 mg to about 300 mg, about 200 mg to about 300 mg, about 210 mg to about 290 mg, about 220 mg to about 280 mg, about 230 mg to about 270 mg, about 240 mg to about 270 mg, about 250 mg about 270 mg, about 260 mg to about 270 mg, about 200 mg to about 400 mg, or about 300 mg to about 400 mg by dry weight. Thus, dried Hericium erinaceus may be present in the compositions in an amount of about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 mg, or any amount therebetween, by dry weight. In some embodiments, dried Hericium erinaceus is present in the compositions in an amount of about 268.9 mg by dry weight.

Dried Wolfiporia extensa may be present in the compositions in an amount from about 10 mg to about 50 mg by dry weight. This includes an amount from about 10 mg to about 20 mg, about 10 mg to about 30 mg, about 10 mg to about 40 mg, about 20 mg to about 25 mg, about 20 mg to about 24 mg, about 20 mg to about 23 mg, about 20 mg to about 22 mg, about 20 mg to about 21 mg, about 20 mg to about 30 mg, about 20 mg to about 40 mg, about 20 mg to about 50 mg, about 30 mg to about 40 mg, about 30 mg to about 50 mg, or about 40 mg to about 50 mg by dry weight. Thus, dried Wolfiporia extensa may be present in the compositions in an amount of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg, or any amount therebetween, by dry weight. In some embodiments, dried Wolfiporia extensa is present in the compositions in an amount of about 20.4 mg by dry weight.

Dried Cordyceps sp. may be present in the compositions in an amount from about 100 mg to about 400 mg by dry weight. This includes an amount from about 100 mg to about 200 mg, about 100 mg to about 300 mg, about 200 mg to about 300 mg, about 210 mg to about 290 mg, about 220 mg to about 280 mg, about 230 mg to about 270 mg, about 240 mg to about 270 mg, about 250 mg about 270 mg, about 260 mg to about 270 mg, about 200 mg to about 400 mg, or about 300 mg to about 400 mg by dry weight. Thus, dried Cordyceps sp. may be present in the compositions in an amount of about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 mg, or any amount therebetween, by dry weight. In some embodiments, dried Cordyceps sp. is present in the compositions in an amount of about 268.9 mg by dry weight. In some embodiments, the dried Cordyceps sp. present in the compositions comprises dried Cordyceps militaris.

Dried Fomes fomentarius may be present in the compositions in an amount from about 10 mg to about 50 mg by dry weight. This includes an amount from about 10 mg to about 20 mg, about 11 mg to about 20 mg, about 12 mg to about 20 mg, about 13 mg to about 20 mg, about 14 mg to about 20 mg, about 15 mg to about 20 mg, about 16 mg to about 20 mg, about 16 mg to about 19 mg, about 16 mg to about 18 mg, about 10 mg to about 30 mg, about 10 mg to about 40 mg, about 20 mg to about 30 mg, about 20 mg to about 40 mg, about 20 mg to about 50 mg, about 30 mg to about 40 mg, about 30 mg to about 50 mg, or about 40 mg to about 50 mg by dry weight. Thus, dried Fomes fomentarius may be present in the compositions in an amount of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg, or any amount therebetween, by dry weight. In some embodiments, dried Fomes fomentarius is present in the compositions in an amount of about 17 mg by dry weight.

Dried Pleurotus ostreatus may be present in the compositions in an amount from about 10 mg to about 50 mg by dry weight. This includes an amount from about 10 mg to about 20 mg, about 11 mg to about 20 mg, about 12 mg to about 20 mg, about 13 mg to about 20 mg, about 14 mg to about 20 mg, about 15 mg to about 20 mg, about 16 mg to about 20 mg, about 16 mg to about 19 mg, about 16 mg to about 18 mg, about 10 mg to about 30 mg, about 10 mg to about 40 mg, about 20 mg to about 30 mg, about 20 mg to about 40 mg, about 20 mg to about 50 mg, about 30 mg to about 40 mg, about 30 mg to about 50 mg, or about 40 mg to about 50 mg by dry weight. Thus, dried Pleurotus ostreatus may be present in the compositions in an amount of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg, or any amount therebetween, by dry weight. In some embodiments, dried Pleurotus ostreatus is present in the compositions in an amount of about 17 mg by dry weight.

Dried Phellinus linteus may be present in the compositions in an amount from about 10 mg to about 50 mg by dry weight. This includes an amount from about 10 mg to about 20 mg, about 10 mg to about 30 mg, about 10 mg to about 40 mg, about 20 mg to about 30 mg, about 20 mg to about 40 mg, about 22 mg to about 38 mg, about 24 mg to about 38 mg, about 26 mg to about 38 mg, about 28 mg to about 38 mg, about 30 mg to about 38 mg, about 32 mg to about 38 mg, about 34 mg to about 38 mg, about 20 mg to about 50 mg, about 30 mg to about 40 mg, about 30 mg to about 50 mg, or about 40 mg to about 50 mg by dry weight. Thus, dried Phellinus linteus may be present in the compositions in an amount of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg, or any amount therebetween, by dry weight. In some embodiments, dried Phellinus linteus is present in the compositions in an amount of about 34 mg by dry weight.

Dried Grifola frondosa may be present in the compositions in an amount from about 10 mg to about 50 mg by dry weight. This includes an amount from about 10 mg to about 20 mg, about 10 mg to about 30 mg, about 10 mg to about 40 mg, about 20 mg to about 30 mg, about 20 mg to about 25 mg, about 20 mg to about 24 mg, about 20 mg to about 23 mg, about 20 mg to about 22 mg, about 20 mg to about 21 mg, about 20 mg to about 40 mg, about 20 mg to about 50 mg, about 30 mg to about 40 mg, about 30 mg to about 50 mg, or about 40 mg to about 50 mg by dry weight. Thus, dried Grifola frondosa may be present in the compositions in an amount of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg, or any amount therebetween, by dry weight. In some embodiments, dried Grifola frondosa is present in the compositions in an amount of about 20.4 mg by dry weight.

Dried Lentinula edodes may be present in the compositions in an amount from about 10 mg to about 50 mg by dry weight. This includes an amount from about 10 mg to about 20 mg, about 10 mg to about 30 mg, about 10 mg to about 40 mg, about 20 mg to about 30 mg, about 20 mg to about 25 mg, about 20 mg to about 24 mg, about 20 mg to about 23 mg, about 20 mg to about 22 mg, about 20 mg to about 21 mg, about 20 mg to about 40 mg, about 20 mg to about 50 mg, about 30 mg to about 40 mg, about 30 mg to about 50 mg, or about 40 mg to about 50 mg by dry weight. Thus, dried Lentinula edodes may be present in the compositions in an amount of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg, or any amount therebetween, by dry weight. In some embodiments, dried Lentinula edodes is present in the compositions in an amount of about 20.4 mg by dry weight.

In exemplary embodiments, the mushroom blends comprise, consist essentially of, or consist of:
about 100 mg to about 400 mg by dry weight of Inonotus obliquus,
about 10 mg to about 50 mg by dry weight of Agaricus subrufescens,
about 100 mg to about 400 mg by dry weight of Laricifomes officinalis,
about 100 mg to about 400 mg by dry weight of Trametes versicolor,
about 100 mg to about 400 mg by dry weight of Ganoderma lingzhi,
about 10 mg to about 50 mg by dry weight of Schizophyllum commune,
about 100 mg to about 400 mg by dry weight of Hericium erinaceus,
about 10 mg to about 50 mg by dry weight of Wolfiporia extensa,
about 100 mg to about 400 mg by dry weight of Cordyceps sp., optionally Cordyceps militaris,
about 10 mg to about 50 mg by dry weight of Fomes fomentarius,
about 10 mg to about 50 mg by dry weight of Pleurotus ostreatus,
about 10 mg to about 50 mg by dry weight of Phellinus linteus,
about 10 mg to about 50 mg by dry weight of Grifola frondosa, and
about 10 mg to about 50 mg by dry weight of Lentinula edodes.

In more specific exemplary embodiments, the mushroom blends comprise, consist essentially of, or consist of:
about 268.9 mg by dry weight of Inonotus obliquus,
about 20.4 mg by dry weight of Agaricus subrufescens,
about 251.9 mg by dry weight of Laricifomes officinalis,
about 255.3 mg by dry weight of Trametes versicolor,
about 268.9 mg by dry weight of Ganoderma lingzhi,
about 17 mg by dry weight of Schizophyllum commune,
about 268.9 mg by dry weight of Hericium erinaceus,
about 20.4 mg by dry weight of Wolfiporia extensa,
about 268.9 mg by dry weight of Cordyceps militaris,
about 17 mg by dry weight of Fomes fomentarius,
about 17 mg by dry weight of Pleurotus ostreatus,
about 34 mg by dry weight of Phellinus linteus,
about 20.4 mg by dry weight of Grifola frondosa, and
about 20.4 mg by dry weight of Lentinula edodes.

In some embodiments, the mushroom blends further comprise a dried mushroom(s) of the genus Psilocybe. In some embodiments, the mushroom is Psilocybe cubensis. When present, dried Psilocybe may be present in the composition in an amount from about 50 mg to about 500 mg by dry weight. This includes an amount of about 50 mg to about 100 mg, about 50 mg to about 200 mg, about 50 mg to about 300 mg, about 50 mg to about 400 mg, about 100 mg to about 200 mg, about 100 mg to about 300 mg, about 100 mg to about 400 mg, about 100 mg to about 500 mg, about 200 mg to about 300 mg, about 200 mg to about 400 mg, about 200 mg to about 500 mg, about 300 mg to about 400 mg, about 300 mg to about 500 mg, or about 400 mg to about 500 mg by dry weight. Thus, dried Psilocybe is present in the composition in an amount of about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 mg, or any amount therebetween, by dry weight. In some embodiments, dried Psilocybe is present in the composition in an amount of about 100 mg by dry weight. The dried Psilocybe present in the composition may comprise Psilocybe cubensis PE6.

Thus, in some exemplary embodiments, the mushroom blends comprise, consist essentially of, or consist of:
about 100 mg to about 400 mg by dry weight of Inonotus obliquus,
about 10 mg to about 50 mg by dry weight of Agaricus subrufescens,
about 100 mg to about 400 mg by dry weight of Laricifomes officinalis,
about 100 mg to about 400 mg by dry weight of Trametes versicolor,
about 100 mg to about 400 mg by dry weight of Ganoderma lingzhi,
about 10 mg to about 50 mg by dry weight of Schizophyllum commune,
about 100 mg to about 400 mg by dry weight of Hericium erinaceus,
about 10 mg to about 50 mg by dry weight of Wolfiporia extensa,
about 100 mg to about 400 mg by dry weight of Cordyceps sp., optionally Cordyceps militaris,
about 10 mg to about 50 mg by dry weight of Fomes fomentarius,
about 10 mg to about 50 mg by dry weight of Pleurotus ostreatus,
about 10 mg to about 50 mg by dry weight of Phellinus linteus,
about 10 mg to about 50 mg by dry weight of Grifola frondosa,
about 10 mg to about 50 mg by dry weight of Lentinula edodes, and (optionally) about 50 mg to about 500 mg by dry weight of Psilocybe cubensis.

In other specific exemplary embodiments, the mushroom blends comprise, consist essentially of, or consist of:
about 268.9 mg by dry weight of Inonotus obliquus,
about 20.4 mg by dry weight of Agaricus subrufescens,
about 251.9 mg by dry weight of Laricifomes officinalis,
about 255.3 mg by dry weight of Trametes versicolor,
about 268.9 mg by dry weight of Ganoderma lingzhi,
about 17 mg by dry weight of Schizophyllum commune,
about 268.9 mg by dry weight of Hericium erinaceus,
about 20.4 mg by dry weight of Wolfiporia extensa,
about 268.9 mg by dry weight of Cordycepsmilitaris,
about 17 mg by dry weight of Fomes fomentarius,
about 17 mg by dry weight of Pleurotus ostreatus,
about 34 mg by dry weight of Phellinus linteus,
about 20.4 mg by dry weight of Grifola frondosa,
about 20.4 mg by dry weight of Lentinula edodes, and (optionally) about 100 mg by dry weight of Psilocybe cubensis.

In some embodiments, the dosage form comprises about or at least about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,500 mg, or about 2,000 mg of a mushroom blend. In some embodiments, the dosage form comprises no more than about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,500 mg, or about 2,000 mg of a mushroom blend.

### Neurotransmitter Activity Modulators

In various embodiments, the compositions can comprise a neurotransmitter activity modulator. The neurotransmitter activity modulator in some embodiments is a serotonergic drug, a dopaminergic drug, an anxiolytic drug, and/or an adrenergic drug.

As used herein, the term “neurotransmitter activity modulator” or neuromodulator refers to a compound or composition that reacts with or influences activity at a neurotransmitter receptor. In some embodiments, the neurotransmitter activity modulator is a serotonergic drug, an adrenergic receptor, a dopamine receptor, a GABAergic receptor, a glutaminergic receptor, a histaminergic receptor, a cholinergic receptor, an opioid receptor, or a glycinergic receptor. In one embodiment, a neurotransmitter activity modulator binds on a neurotransmitter receptor. In one embodiment, a neurotransmitter activity modulator indirectly affects a neurotransmitter receptor, e.g., via interactions affecting the reactivity of other molecules at a neurotransmitter receptor. In one embodiment, a neurotransmitter activity modulator is an agonist. In one embodiment, a neurotransmitter activity modulator is an antagonist. In one embodiment, a neurotransmitter activity modulator acts (either directly or indirectly) at more than one type of neurotransmitter receptor. In some embodiments, the neurotransmitter activity modulator is a compound that influences activity at one or more of the following receptors: serotonin receptor, adrenergic receptor, dopamine receptor, GABA receptor, glutaminergic receptor, histaminergic receptor, cholinergic receptor, opioid receptor, glycinergic receptor, alpha 1 and alpha 2 receptors, sigma 1 and sigma 2 receptors, and muscarinic receptors.

Non-limiting examples of neurotransmitter activity modulators include aripiprazole, bupropion, citalopram, clomipramine, dextroamphetamine, duloxetine, escitalopram, fluoxetine, fluvoxamine, milnacipran, mirtazapine, paroxetine, quetiapine, reboxetine, risperidone, sertraline, and venlafaxine.

As used herein, the term “serotonergic drug” refers to a compound that binds to, blocks, or otherwise influences activity at a serotonin receptor. In one embodiment, a serotonergic drug binds to a serotonin receptor. In one embodiment, a serotonergic drug indirectly affects a serotonin receptor, e.g., via interactions affecting the reactivity of other molecules at the serotonin receptor. In one embodiment, a serotonergic drug is an agonist, e.g., a compound activating a serotonin receptor. In one embodiment, a serotonergic drug is an antagonist, e.g., a compound binding but not activating a serotonin receptor, e.g., blocking a receptor. In one embodiment, a serotonergic drug is an effector molecule, e.g., a compound binding to an enzyme for allosteric regulation. In one embodiment, a serotonergic drug acts (either directly or indirectly) at more than one type of receptor (e.g., 5HT, dopamine, adrenergic, acetylcholine, etc.). In one embodiment, a serotonergic drug is an antidepressant. In one embodiment, a serotonergic drug is an anxiolytic. In some embodiments, a serotonergic drug is a tricyclic antidepressant (TCA). In one embodiment, a serotonergic drug is a selective serotonin reuptake inhibitor (SSRI). In one embodiment, a serotonergic drug is a selective serotonin norepinephrine reuptake inhibitor (SNRI).

Some exemplary serotonergic drugs include the following molecules: 4-hydroxy-N-methyltryptamine (also known as 3-[2-(methylamino)ethyl]-1H-indol-4-ol), aeruginascin (also known as [3-[2-(trimethylazaniumyl)ethyl]-1H-indol-4-yl] hydrogen phosphate), baeocystin (also known as [3-[2-(methylamino)ethyl]-1H-indol-4-yl] dihydrogen phosphate), bufotenidine (also known as 3-[2-(trimethylazaniumyl)ethyl]-1H-indol-5-olate), bufotenin (also known as 3-[2-(dimethylamino)ethyl]-1H-indol-5-ol), ethocybin (also known as [3-[2-(diethylamino)ethyl]-1H-indol-4-yl] dihydrogen phosphate), norbaeocystin (also known as [3-(2-aminoethyl)-1H-indol-4-yl] dihydrogen phosphate), norpsilocin, psilocin (also known as 3-[2-(dimethylamino)ethyl]-1H-indol-4-ol), psilocybin (also known as [3-[2-(dimethylamino)ethyl]-1H-indol-4-yl] dihydrogen phosphate), serotonin (also known as 3-(2-aminoethyl)-1H-indol-5-ol), 1P-LSD (also known as (6aR,9R)—N,N-diethyl-7-methyl-4-propanoyl-6,6a,8,9-tetrahydroindolo[4,3-fg]quinoline-9-carboxamide), ALD-52 (also known as (6aR,9R)-4-acetyl-N,N-diethyl-7-methyl-6,6a,8,9-tetrahydroindolo[4,3-fg]quinoline-9-carboxamide), AL-LAD (also known as (6aR,9R)—N,N-diethyl-7-prop-2-enyl-6,6a,8,9-tetrahydro-4H-indolo[4,3-fg]quinoline-9-carboxamide), BU-LAD (also known as (6aR,9R)-7-butyl-N,N-diethyl-6,6a,8,9-tetrahydro-4H-indolo[4,3-fg]quinoline-9-carboxamide), DAL (also known as (6aR,9R)-7-methyl-N,N-bis(prop-2-enyl)-6,6a,8,9-tetrahydro-4H-indolo[4,3-fg]quinoline-9-carboxamide), DAM-57 (also known as (6aR,9R)—N,N,7-trimethyl-6,6a,8,9-tetrahydro-4H-indolo[4,3-fg]quinoline-9-carboxamide), EIPLA (also known as (6aR,9R)—N-ethyl-7-methyl-N-propan-2-yl-6,6a,8,9-tetrahydro-4H-indolo[4,3-fg]quinoline-9-carboxamide), ETH-LAD (also known as (6aR,9R)—N,N,7-triethyl-6,6a,8,9-tetrahydro-4H-indolo[4,3-fg]quinoline-9-carboxamide), LAE-32 (also known as (6aR,9R)—N-ethyl-7-methyl-6,6a,8,9-tetrahydro-4H-indolo[4,3-fg]quinoline-9-carboxamide), LPD-824 (also known as [(6aR,9R)-7-methyl-6,6a,8,9-tetrahydro-4H-indolo[4,3-fg]quinoline-9-yl]-pyrrolidin-1-ylmethanone), LSB (also known as (6aR,9R)—N-butan-2-yl-7-methyl-6,6a,8,9-tetrahydro-4H-indolo[4,3-fg]quinoline-9-carboxamide), LSA (also known as (6aR,9R)-7-methyl-6,6a,8,9-tetrahydro-4H-indolo[4,3-fg]quinoline-9-carboxamide), LSD-25 (also known as (6aR,9R)—N,N-diethyl-7-methyl-6,6a,8,9-tetrahydro-4H-indolo[4,3-fg]quinoline-9-carboxamide), LSD-PiP (also known as (7-methyl-6,6a,8,9-tetrahydro-4H-indolo[4,3-fg]quinoline-9-yl)-piperidin-1-ylmethanone), LSM-775 (also known as [(6aR,9R)-7-methyl-6,6a,8,9-tetrahydro-4H-indolo[4,3-fg]quinoline-9-yl]-morpholin-4-ylmethanone), LSP (also known as (6aR,9R)-7-methyl-N-pentan-3-yl-6,6a,8,9-tetrahydro-4H-indolo[4,3-fg]quinoline-9-carboxamide), LSZ (also known as [(6aR,9R)-7-methyl-6,6a,8,9-tetrahydro-4H-indolo[4,3-fg]quinoline-9-yl]-[(2S,4S)-2,4-dimethylazetidin-1-yl]methanone), methergine (also known as (6aR,9R)—N-(1-hydroxybutan-2-yl)-7-methyl-6,6a,8,9-tetrahydro-4H-indolo[4,3-fg]quinoline-9-carboxamide), MiPLA (also known as (6aR,9R)—N,7-dimethyl-N-propan-2-yl-6,6a,8,9-tetrahydro-4H-indolo[4,3-fg]quinoline-9-carboxamide), NDTDI, PARGY-LAD, PRO-LAD (also known as (6aR,9R)—N,N-diethyl-7-propyl-6,6a,8,9-tetrahydro-4H-indolo[4,3-fg]quinoline-9-carboxamide), 2-Me-DET (also known as N,N-diethyl-2-(2-methyl-1H-indol-3-yl)ethanamine), 2-Me-DMT (also known as N,N-dimethyl-2-(2-methyl-1H-indol-3-yl)ethanamine), 2,alpha-DMT (also known as 1-(2-methyl-1H-indol-3-yl)propan-2-amine), 4-AcO-DALT (also known as [3-[2-[bis(prop-2-enyl)amino]ethyl]-1H-indol-4-yl] acetate), 4-AcO-DET (also known as [3-[2-(diethylamino)ethyl]-1H-indol-4-yl] acetate), 4-AcO-DIPT (also known as 3-[2-(Diisopropylamino)ethyl]-1H-indol-4-yl acetate), 4-AcO-DMT (also known as [3-[2-(dimethylamino)ethyl]-1H-indol-4-yl] acetate), 4-AcO-DPT (also known as [3-[2-(dipropylamino)ethyl]-1H-indol-4-yl] acetate), 4-AcO-EPT (also known as 3-{2-[Ethyl(propyl)amino]ethyl}-1H-indol-4-yl acetate), 4-AcO-MET (also known as [3-[2-[ethyl(methyl)amino]ethyl]-1H-indol-4-yl] acetate), 4-AcO-MIPT (also known as [3-[2-[methyl(propan-2-yl)amino]ethyl]-1H-indol-4-yl] acetate), 4-AcO-MPT, 4-HO-DBT (also known as 3-[2-(dibutylamino)ethyl]-1H-indol-4-ol), 4-HO-DET (also known as 3-[2-(diethylamino)ethyl]-1H-indol-4-ol), 4-HO-DIPT (also known as 3-[2-[di(propan-2-yl)amino]ethyl]-1H-indol-4-ol), 4-HO-DPT (also known as 3-[2-(dipropylamino)ethyl]-1H-indol-4-ol), 4-HO-EPT, 4-HO-MCPT, 4-HO-MET (also known as 3-[2-[ethyl(methyl)amino]ethyl]-1H-indol-4-ol), 4-HO-MIPT (also known as 3-[2-[methyl(propan-2-yl)amino]ethyl]-1H-indol-4-ol), 4-HO-MPMI (also known as 3-[(1-methylpyrrolidin-2-yl)methyl]-1H-indol-4-ol), 4-HO-MPT (also known as 3-[2-[methyl(propyl)amino]ethyl]-1H-indol-4-ol), 4-HO-pyr-T (also known as 3-(2-pyrrolidin-1-ylethyl)-1H-indol-4-ol), 4-MeO-MIPT (also known as N-[2-(4-methoxy-1H-indol-3-yl)ethyl]-N-methylpropan-2-amine), 4,5-MDO-DIPT (also known as N-[2-(6H-[1,3]dioxolo[4,5-e]indol-8-yl)ethyl]-N-propan-2-ylpropan-2-amine), 4,5-MDO-DMT (also known as 2-(6H-[1,3]dioxolo[4,5-e]indol-8-yl)-N,N-dimethylethanamine), 5-BROMO-DMT (also known as 2-(5-bromo-1H-indol-3-yl)-N,N-dimethylethanamine), 5-chloro-alpha-MT (also known as 1-(5-chloro-1H-indol-3-yl)propan-2-amine), 5-fluoro-AMT (also known as 1-(5-fluoro-1H-indol-3-yl)propan-2-amine), 5-MeO-AET (also known as 1-(5-methoxy-1H-indol-3-yl)butan-2-amine), 5-MEO-AMT (also known as 1-(5-methoxy-1H-indol-3-yl)propan-2-amine), 5-MeO-DALT (also known as N-[2-(5-methoxy-1H-indol-3-yl)ethyl]-N-prop-2-enylprop-2-en-1-amine), 5-MeO-DET (also known as N,N-diethyl-2-(5-methoxy-1H-indol-3-yl)ethanamine), 5-MeO-DiPT (also known as N-[2-(5-methoxy-1H-indol-3-yl)ethyl]-N-propan-2-ylpropan-2-amine), 5-MeO-DMT (also known as 2-(5-methoxy-1H-indol-3-yl)-N,N-dimethylethanamine), 5-MeO-DPT (also known as N-[2-(5-methoxy-1H-indol-3-yl)ethyl]-N-propylpropan-1-amine), 5-MeO-EiPT (also known as N-ethyl-N-[2-(5-methoxy-1H-indol-3-yl)ethyl]propan-2-amine), 5-MeO-MALT (also known as N-[2-(5-Methoxy-1H-indol-3-yl)ethyl]-N-methylprop-2-en-1-amine), 5-MeO-MiPT (also known as N-[2-(5-methoxy-1H-indol-3-yl)ethyl]-N-methylpropan-2-amine), 5-MeO-NMT (also known as 2-(5-methoxy-1H-indol-3-yl)-N-methylethanamine; hydrochloride), 5-MeO-pyr-T (also known as 4-fluoro-5-methoxy-3-(2-pyrrolidin-1-ylethyl)-1H-indole), 5-MeO-TMT (also known as 2-(5-methoxy-2-methyl-1H-indol-3-yl)-N,N-dimethylethanamine), 5-MeS-DMT (also known as N,N-dimethyl-2-(5-methylsulfanyl-1H-indol-3-yl)ethanamine), 5,6-MDO-DIPT (also known as N-[2-(5H-[1,3]dioxolo[4,5-f|indol-7-yl)ethyl]-N-propan-2-ylpropan-2-amine), 5,6-MDO-DMT (also known as 2-(5H-[1,3]dioxolo[4,5-f]indol-7-yl)-N,N-dimethylethanamine), 5,6-MDO-MIPT (also known as N-[2-(5H-[1,3]dioxolo[4,5-f|indol-7-yl)ethyl]-N-methylpropan-2-amine), 5,6-MeO-MIPT (also known as N-[2-(5,6-dimethoxy-1H-indol-3-yl)ethyl]-N-methylpropan-2-amine), 5,N,N-TMT (also known as N,N-dimethyl-2-(5-methyl-1H-indol-3-yl)ethanamine), 6-MeO-THH (also known as 6-methoxy-1-methyl-2,3,4,9-tetrahydro-1H-pyrido[3,4-b]indole), alpha-ET (also known as 1-(1H-indol-3-yl)butan-2-amine), alpha-MT (also known as 1-(1H-indol-3-yl)propan-2-amine), alpha-TMT (also known as 1-(1H-indol-3-yl)-N,N-dimethylpropan-2-amine), alpha, N-DMT (also known as 2-(1H-indol-3-yl)-N,N-dimethylethanamine), alpha, N,G-TMS (also known as 1-(5-methoxy-1H-indol-3-yl)-N-methylpropan-2-amine), alpha, G-DMS (also known as 1-(5-methoxy-1H-indol-3-yl)propan-2-amine), DALT (also known as N-[2-(1H-indol-3-yl)ethyl]-N-prop-2-enylprop-2-en-1-amine), DBT (also known as N-butyl-N-[2-(1H-indol-3-yl)ethyl]butan-1-amine), DET (also known as N,N-diethyl-2-(1H-indol-3-yl)ethanamine), DiPT (also known as N-[2-(5-methoxy-1H-indol-3-yl)ethyl]-N-propan-2-ylpropan-2-amine), DMT (also known as 2-(1H-indol-3-yl)-N,N-dimethylethanamine), DPT (also known as N-[2-(1H-indol-3-yl)ethyl]-N-propylpropan-1-amine), EiPT (also known as N-ethyl-N-[2-(1H-indol-3-yl)ethyl]propan-2-amine), Harmaline (also known as 7-methoxy-1-methyl-3,4-dihydro-2H-pyrido[3,4-b]indole), Harmine (also known as 7-methoxy-1-methyl-9H-pyrido[3,4-b]indole), MALT, MBT (also known as 3H-1,3-benzothiazole-2-thione), Melatonin (also known as N-[2-(5-methoxy-1H-indol-3-yl)ethyl]acetamide), MET (also known as N-ethyl-2-(1H-indol-3-yl)-N-methylethanamine), MiPT (also known as N-[2-(1H-indol-3-yl)ethyl]-N-methylpropan-2-amine), MPT (also known as 3-[2-[methyl(propyl)amino]ethyl]-1H-indol-4-ol), NET (also known as N-ethyl-2-(1H-indol-3-yl)ethanamine), NMT (also known as 2-(1H-indol-3-yl)-N-methylethanamine), PiPT (also known as N-[2-(1H-indol-3-yl)ethyl]-N-propan-2-ylpropan-1-amine), pyr-T (also known as 3-(2-pyrrolidin-1-ylethyl)-1H-indole), T (also known as 2-(1H-indol-3-yl)ethanamine), Tetrahydroharmine (also known as 7-methoxy-1-methyl-2,3,4,9-tetrahydro-1H-pyrido[3,4-b]indole), 2-Br-4,5-MDA (also known as 1-(6-bromo-1,3-benzodioxol-5-yl)propan-2-amine), 2-TIM (also known as 2-(3,4-dimethoxy-2-methylsulfanylphenyl)ethanamine), 2-TOET (also known as 1-(4-ethyl-5-methoxy-2-methylsulfanylphenyl)propan-2-amine), 2-TOM (also known as 1-(5-methoxy-4-methyl-2-methylsulfanylphenyl)propan-2-amine), 2,4-DMA (also known as 1-(2,4-dimethoxyphenyl)propan-2-amine), 2,5-DMA (also known as 1-(2,5-dimethoxyphenyl)propan-2-amine), 2C-B (also known as 2-(4-bromo-2,5-dimethoxyphenyl)ethanamine), 2C-C (also known as 2-(4-chloro-2,5-dimethoxyphenyl)ethanamine), 2C-D (also known as 2-(2,5-dimethoxy-4-methylphenyl)ethanamine), 2C-E (also known as 2-(4-ethyl-2,5-dimethoxyphenyl)ethanamine), 2C-F (also known as 2-(4-fluoro-2,5-dimethoxyphenyl)ethanamine), 2C-G (also known as 2-(2,5-dimethoxy-3,4-dimethylphenyl)ethanamine), 2C-G-3 (also known as 2-(4,7-dimethoxy-2,3-dihydro-1H-inden-5-yl)ethanamine), 2C-G-4 (also known as 2-(1,4-dimethoxy-5,6,7,8-tetrahydronaphthalen-2-yl)ethanamine), 2C-G-5 (also known as CAS 207740-20-3), 2C-G-N (also known as 2-(1,4-dimethoxynaphthalen-2-yl)ethanamine), 2C-H (also known as 2-(2,5-dimethoxyphenyl)ethanamine), 2C-I (also known as 2-(4-iodo-2,5-dimethoxyphenyl)ethanamine), 2C-N (also known as 2-(2,5-dimethoxy-4-nitrophenyl)ethanamine), 2C-0-4 (also known as 2-(2,5-dimethoxy-4-propan-2-yloxyphenyl)ethanamine), 2C-P (also known as 2-(2,5-dimethoxy-4-propylphenyl)ethanamine), 2C-SE (also known as 2-(2,5-dimethoxy-4-methylselanylphenyl)ethanamine), 2C-T (also known as 2-(2,5-dimethoxy-4-methylsulfanylphenyl)ethanamine), 2C-T-13 (also known as 2-[2,5-dimethoxy-4-(2-methoxyethylsulfanyl)phenyl]ethanamine), 2C-T-15 (also known as 2-(4-cyclopropylsulfanyl-2,5-dimethoxyphenyl)ethanamine), 2C-T-17 (also known as 2-(4-butan-2-ylsulfanyl-2,5-dimethoxyphenyl)ethanamine), 2C-T-2 (also known as 2-(4-ethylsulfanyl-2,5-dimethoxyphenyl)ethanamine), 2C-T-21 (also known as 2-[4-(2-fluoroethylsulfanyl)-2,5-dimethoxyphenyl]ethanamine), 2C-T-4 (also known as 2-(2,5-dimethoxy-4-propan-2-ylsulfanylphenyl)ethanamine), 2C-T-7 (also known as 2-(2,5-dimethoxy-4-propylsulfanylphenyl)ethanamine), 2C-T-8 (also known as 2-[4-(cyclopropylmethylsulfanyl)-2,5-dimethoxyphenyl]ethanamine), 2C-T-9 (also known as 2-(4-butylsulfanyl-2,5-dimethoxyphenyl)ethanamine), 2C-TFM (also known as 2-[2,5-dimethoxy-4-(trifluoromethyl)phenyl]ethanamine), 2T-MMDA-3a (also known as 1-(4-methylsulfanyl-1,3-benzodioxol-5-yl)propan-2-amine), 3-T-TRIS (also known as 2-(3,4-diethoxy-5-ethylsulfanylphenyl)ethanamine), 3-TASB (also known as 2-(3-ethoxy-4-ethylsulfanyl-5-methoxyphenyl)ethanamine), 3-TE (also known as 2-(4-ethoxy-3-methoxy-5-methylsulfanylphenyl)ethanamine), 3-TIM (also known as 2-(2,4-dimethoxy-3-methylsulfanylphenyl)ethanamine), 3-TM (also known as 2-(3,4-dimethoxy-5-methylsulfanylphenyl)ethanamine), 3-TME (also known as 2-(3-ethylsulfanyl-4,5-dimethoxyphenyl)ethanamine), 3-TSB (also known as 2-(3-ethoxy-5-ethylsulfanyl-4-methoxyphenyl)ethanamine), 3,4-DMA (also known as 1-(3,4-dimethoxyphenyl)propan-2-amine), 3C-BZ (also known as 1-(3,5-dimethoxy-4-phenylmethoxyphenyl)propan-2-amine), 3C-E (also known as 1-(4-ethoxy-3, 5-dimethoxyphenyl)propan-2-amine), 4-Br-3,5-DMA (also known as 1-(4-bromo-3,5-dimethoxyphenyl)propan-2-amine), 4-D (also known as CAS 1020518-87-9), 4-MA (also known as 1-(4-methoxyphenyl)propan-2-amine), 4-T-TRIS (also known as 2-(3,5-diethoxy-4-ethylsulfanylphenyl)ethanamine), 4-TASB (also known as 2-(3-ethoxy-4-ethylsulfanyl-5-methoxyphenyl)ethanamine), 4-TE (also known as 2-(4-ethylsulfanyl-3,5-dimethoxyphenyl)ethanamine), 4-TIM (also known as 2-(2,3-dimethoxy-4-methylsulfanylphenyl)ethanamine), 4-TM (also known as 2-(3,5-dimethoxy-4-methylsulfanylphenyl)ethanamine), 4-TME (also known as 2-(3-ethoxy-5-methoxy-4-methylsulfanylphenyl)ethanamine), 4-TSB (also known as 2-(3, 5-diethoxy-4-methylsulfanylphenyl)ethanamine), 4T-MMDA-2 (also known as 1-(5-methoxy-1,3-benzoxathiol-6-yl)propan-2-amine), 5-TASB (also known as 2-(3, 4-diethoxy-5-methylsulfanylphenyl)ethanamine), 5-TME (also known as 2-(3-ethoxy-4-methoxy-5-methylsulfanylphenyl)ethanamine), 5-TOET (also known as 1-(4-ethyl-2-methoxy-5-methylsulfanylphenyl)propan-2-amine), 5-TOM (also known as 1-(2-methoxy-4-methyl-5-methylsulfanylphenyl)propan-2-amine), 25B-NBF (also known as 2-(4-bromo-2,5-dimethoxyphenyl)-N-[(2-fluorophenyl)methyl]ethanamine), 25B-NBOH (also known as 2-[[2-(4-bromo-2,5-dimethoxyphenyl)ethylamino]methyl]phenol), 25B-NBOMe (also known as 2-(4-bromo-2,5-dimethoxyphenyl)-N-[(2-methoxyphenyl)methyl]ethanamine), 25C-NB30Me (also known as 2-(4-chloro-2,5-dimethoxyphenyl)-N-[(3-methoxyphenyl)methyl]ethanamine), 25C-NB40Me (also known as 2-(4-chloro-2,5-dimethoxyphenyl)-N-[(4-methoxyphenyl)methyl]ethanamine), 25C-NBF (also known as 2-(4-chloro-2,5-dimethoxyphenyl)-N-[(2-fluorophenyl)methyl]ethanamine), 25C-NBOH (also known as 2-[[2-(4-chloro-2,5-dimethoxyphenyl)ethylamino]methyl]phenol), 25C-NBOMe (also known as 2-(4-chloro-2,5-dimethoxyphenyl)-N-[(2-methoxyphenyl)methyl]ethanamine), 25CN-NBOH (also known as 4-[2-[(2-hydroxyphenyl)methylamino]ethyl]-2,5-dimethoxybenzonitrile), 25CN-NBOMe (also known as CAS 1354632-16-8), 25D-NBOMe (also known as 2-(2,5-dimethoxy-4-methylphenyl)-N-[(2-methoxyphenyl)methyl]ethanamine), 25E-NBOMe (also known as 2-(4-ethyl-2,5-dimethoxyphenyl)-N-[(2-methoxyphenyl)methyl]ethanamine), 25G-NBOMe (also known as 2-(2,5-dimethoxy-3,4-dimethylphenyl)-N-[(2-methoxyphenyl)methyl]ethanamine), 25H-NBOMe (also known as 2-(2,5-dimethoxyphenyl)-N-[(2-methoxyphenyl)methyl]ethanamine), 25I-NB34MD (also known as N-(1,3-benzodioxol-5-ylmethyl)-2-(4-iodo-2,5-dimethoxyphenyl)ethanamine), 25I-NB30Me (also known as 2-(4-iodo-2,5-dimethoxyphenyl)-N-[(3-methoxyphenyl)methyl]ethanamine), 25I-NB40Me (also known as 2-(4-iodo-2,5-dimethoxyphenyl)-N-[(4-methoxyphenyl)methyl]ethanamine), 25I-NBF (also known as N-[(2-fluorophenyl)methyl]-2-(4-iodo-2,5-dimethoxyphenyl)ethanamine), 25I-NBMD (also known as N-(1,3-benzodioxol-4-ylmethyl)-2-(4-iodo-2,5-dimethoxyphenyl)ethanamine), 25I-NBOH (also known as 2-[[2-(4-iodo-2,5-dimethoxyphenyl)ethylamino]methyl]phenol), 251-NBOMe (also known as 2-(4-iodo-2,5-dimethoxyphenyl)-N-[(2-methoxyphenyl)methyl]ethanamine), 25iP-NBOMe (also known as 2-(2,5-dimethoxy-4-propan-2-ylphenyl)-N-[(2-methoxyphenyl)methyl]ethanamine), 25N-NBOMe (also known as 2-(2,5-dimethoxy-4-nitrophenyl)-N-[(2-methoxyphenyl)methyl]ethanamine), 25P-NBOMe (also known as 2-(2,5-dimethoxy-4-propylphenyl)-N-[(2-methoxyphenyl)methyl]ethanamine), 25TFM-NBOMe (also known as 2-[2,5-dimethoxy-4-(trifluoromethyl)phenyl]-N-[(2-methoxyphenyl)methyl]ethanamine), 2CBCB-NBOMe (also known as 1-[(7R)-3-bromo-2,5-dimethoxy-7-bicyclo[4.2.0]octa-1(6),2,4-trienyl]-N-[(2-methoxyphenyl)methyl]methanamine), 2CBFly-NBOMe (also known as 2-(4-bromo-2,3,6,7-tetrahydrofuro[2,3-f|[1]benzofuran-8-yl)-N-[(2-methoxyphenyl)methyl]ethanamine), AEM (also known as 1-(3,4,5-trimethoxyphenyl)butan-2-amine), AL (also known as 2-(3,5-dimethoxy-4-prop-2-enoxyphenyl)ethanamine), ALEPH (also known as 1-(2,5-dimethoxy-4-methylsulfanylphenyl)propan-2-amine; hydrochloride), ALEPH-2 (also known as 1-(4-ethylsulfanyl-2,5-dimethoxyphenyl)propan-2-amine), ALEPH-4 (also known as 1-(2,5-dimethoxy-4-propan-2-ylsulfanylphenyl)propan-2-amine), ALEPH-6 (also known as 1-(2,5-dimethoxy-4-phenylsulfanylphenyl)propan-2-amine), ALEPH-7 (also known as 1-(2,5-dimethoxy-4-propylsulfanylphenyl)propan-2-amine), ARIADNE (also known as (2R)-1-(2,5-dimethoxy-4-methylphenyl)butan-2-amine), ASB (also known as 2-(3,4-diethoxy-5-methoxyphenyl)ethanamine), B (also known as 2-(4-butoxy-3,5-dimethoxyphenyl)ethanamine), BEATRICE (also known as 1-(2,5-dimethoxy-4-methylphenyl)-N-methylpropan-2-amine), beta-D (also known as 2,2-dideuterio-2-(3,4,5-trimethoxyphenyl)ethanamine), BIS-TOM (also known as 1-[4-methyl-2,5-bis(methylsulfanyl)phenyl]propan-2-amine), bk-2C-B (also known as 2-amino-1-(4-bromo-2,5-dimethoxyphenyl)ethanone), BOB (also known as 2-(4-bromo-2,5-dimethoxyphenyl)-2-methoxyethanamine), BOD (also known as 2-(2,5-dimethoxy-4-methylphenyl)-2-methoxyethanamine), BOH (also known as 2-(1,3-benzodioxol-5-yl)-2-methoxyethanamine), BOHD (also known as 2-amino-1-(2,5-dimethoxy-4-methylphenyl)ethanol), BOM (also known as 2-methoxy-2-(3,4,5-trimethoxyphenyl)ethanamine), bromo-dragonFLY (also known as 1-(4-bromofuro[2,3-f|[1]benzofuran-8-yl)propan-2-amine), butylone (also known as 1-(1,3-benzodioxol-5-yl)-2-(methylamino)butan-1-one), CPM (also known as 2-[4-(cyclopropylmethoxy)-3,5-dimethoxyphenyl]ethanamine), DESOXY (also known as 2-(3,5-dimethoxy-4-methylphenyl)ethanamine), DMCPA (also known as 2-(2,5-dimethoxy-4-methylphenyl)cyclopropan-1-amine), DME (also known as 2-amino-1-(3,4-dimethoxyphenyl)ethanol), DMMDA (also known as 1-(4,7-dimethoxy-1,3-benzodioxol-5-yl)propan-2-amine), DMMDA-2 (also known as 1-(6,7-dimethoxy-1,3-benzodioxol-5-yl)propan-2-amine), DMPEA (also known as 2-(3,4-dimethoxyphenyl)ethanamine), DOAM (also known as 1-(2,5-dimethoxy-4-pentylphenyl)propan-2-amine), DOB (also known as 1-(4-bromo-2,5-dimethoxyphenyl)propan-2-amine), DOBU (also known as 1-(4-butyl-2,5-dimethoxyphenyl)propan-2-amine), DOC (also known as 1-(4-chloro-2,5-dimethoxyphenyl)propan-2-amine), DOEF (also known as 1-[4-(2-fluoroethyl)-2,5-dimethoxyphenyl]propan-2-amine), DOET (also known as 1-(4-ethyl-2,5-dimethoxyphenyl)propan-2-amine), DOF (also known as 1-(4-fluoro-2,5-dimethoxyphenyl)propan-2-amine), DOI (also known as 1-(4-iodo-2,5-dimethoxyphenyl)propan-2-amine), DOM (also known as 1-(2,5-dimethoxy-4-methylphenyl)propan-2-amine), DON (also known as 1-(2,5-dimethoxy-4-nitrophenyl)propan-2-amine), DOPR (also known as 1-(2,5-dimethoxy-4-propylphenyl)propan-2-amine), DOTFM (also known as 1-[2,5-dimethoxy-4-(trifluoromethyl)phenyl]propan-2-amine), E (also known as 2-(4-ethoxy-3,5-dimethoxyphenyl)ethanamine), EBDP (also known as 1-(1,3-benzodioxol-5-yl)-N-ethylpentan-2-amine), EEE (also known as 1-(2,4,5-triethoxyphenyl)propan-2-amine), EEM (also known as 1-(2,4-diethoxy-5-methoxyphenyl)propan-2-amine), EME (also known as 1-(2,5-diethoxy-4-methoxyphenyl)propan-2-amine), EMM (also known as 1-(2-ethoxy-4, 5-dimethoxyphenyl)propan-2-amine), ETHYL-J (also known as 1-(1,3-benzodioxol-5-yl)-N-ethylbutan-2-amine), ETHYL-K (also known as 1-(1,3-benzodioxol-5-yl)-N-ethylpentan-2-amine), F-2 (also known as 1-(5-methoxy-2-methyl-2,3-dihydro-1-benzofuran-6-yl)propan-2-amine), F-22 (also known as 1-(5-methoxy-2,2-dimethyl-3H-1-benzofuran-6-yl)propan-2-amine), FLEA (also known as N-[1-(1,3-benzodioxol-5-yl)propan-2-yl]-N-methylhydroxylamine), G-3 (also known as 1-(4,7-dimethoxy-2,3-dihydro-1H-inden-5-yl)propan-2-amine), G-4 (also known as 1-(1,4-dimethoxy-5,6,7,8-tetrahydronaphthalen-2-yl)propan-2-amine), G-5 (also known as 3,6-dimethoxy-4-(2-aminopropyl)benzonorbornane), G-N (also known as 1-(1,4-dimethoxynaphthalen-2-yl)propan-2-amine), GANESHA (also known as 1-(2,5-dimethoxy-3,4-dimethylphenyl)propan-2-amine), HOT-17 (also known as N-[2-(4-butan-2-ylsulfanyl-2,5-dimethoxyphenyl)ethyl]hydroxylamine), HOT-2 (also known as N-[2-(4-ethylsulfanyl-2,5-dimethoxyphenyl)ethyl]hydroxylamine), HOT-7 (also known as N-[2-(2,5-dimethoxy-4-propylsulfanylphenyl)ethyl]hydroxylamine), IDNNA (also known as 1-(4-iodo-2,5-dimethoxyphenyl)-N,N-dimethylpropan-2-amine), IM (also known as 2-(2,3,4-trimethoxyphenyl)ethanamine), IP (also known as 2-(3,5-dimethoxy-4-propan-2-yloxyphenyl)ethanamine), IRIS (also known as 1-(5-ethoxy-2-methoxy-4-methylphenyl)propan-2-amine), J (also known as 1-(1, 3-benzodioxol-5-yl)butan-2-amine), jimscaline (also known as [(1R)-4,5,6-trimethoxy-2,3-dihydro-1H-inden-1-yl]methanamine), LOPHOPHINE (also known as 2-(7-methoxy-1,3-benzodioxol-5-yl)ethanamine), M (also known as 2-(3,4,5-trimethoxyphenyl)ethanamine), MADAM-6 (also known as N-methyl-1-(6-methyl-1,3-benzodioxol-5-yl)propan-2-amine), MAL (also known as 2-[3,5-dimethoxy-4-(2-methylprop-2-enoxy)phenyl]ethanamine), MDA (also known as 1-(1,3-benzodioxol-5-yl)propan-2-amine), MDAL (also known as 1-(1,3-benzodioxol-5-yl)-N-prop-2-enylpropan-2-amine), MDBU (also known as N-[1-(1,3-benzodioxol-5-yl)propan-2-yl]butan-1-amine), MDBZ (also known as 1-(1,3-benzodioxol-5-yl)-N-benzylpropan-2-amine), MDCPM (also known as 1-(3a,7a-dihydro-1,3-benzodioxol-5-yl)-N-(cyclopropylmethyl)propan-2-amine), MDDM (also known as 1-(1,3-benzodioxol-5-yl)-N,N-dimethylpropan-2-amine), MDE (also known as 1-(1,3-benzodioxol-5-yl)-N-ethylpropan-2-amine), MDHOET (also known as 2-[1-(1,3-benzodioxol-5-yl)propan-2-ylamino]ethanol), MDIP (also known as 1-(1,3-benzodioxol-5-yl)-N-propan-2-ylpropan-2-amine), MDMA (also known as 1-(1,3-benzodioxol-5-yl)-N-methylpropan-2-amine), MDMC (also known as 1-(2,3-dihydro-1,4-benzodioxin-6-yl)-N-methylpropan-2-amine), MDMEO (also known as 1-(1,3-benzodioxol-5-yl)-N-methoxypropan-2-amine), MDMEOET (also known as 1-(1,3-benzodioxol-5-yl)-N-(2-methoxyethyl)propan-2-amine), MDMP (also known as 1-(1,3-benzodioxol-5-yl)-N,2-dimethylpropan-2-amine), MDOH (also known as N-[1-(1,3-benzodioxol-5-yl)propan-2-yl]hydroxylamine), MDPEA (also known as 2-(1,3-benzodioxol-5-yl)ethanamine), MDPH (also known as 1-(1,3-benzodioxol-5-yl)-2-methylpropan-2-amine), MDPL (also known as 1-(1,3-benzodioxol-5-yl)-N-prop-2-ynylpropan-2-amine), MDPR (also known as 1-(1,3-benzodioxol-5-yl)-N-propylpropan-2-amine), ME (also known as 2-(3-ethoxy-4,5-dimethoxyphenyl)ethanamine), MEDA (also known as 1-(5-methoxy-2,3-dihydro-1,4-benzodioxin-7-yl)propan-2-amine), MEE (also known as 1-(4,5-diethoxy-2-methoxyphenyl)propan-2-amine), MEM (also known as 1-(4-ethoxy-2,5-dimethoxyphenyl)propan-2-amine), MEPEA (also known as 2-(4-ethoxy-3-methoxyphenyl)ethanamine), META-DOB (also known as 1-(5-bromo-2,4-dimethoxyphenyl)propan-2-amine), META-DOT (also known as 1-(2,4-dimethoxy-5-methylsulfanylphenyl)propan-2-amine), METHYL-DMA (also known as 1-(2,5-dimethoxyphenyl)-N-methylpropan-2-amine), METHYL-DOB (also known as 1-(4-bromo-2,5-dimethoxyphenyl)-N-methylpropan-2-amine), METHYL-J (also known as 1-(1,3-benzodioxol-5-yl)-N-methylbutan-2-amine), METHYL-K (also known as 1-(1,3-benzodioxol-5-yl)-N-methylpentan-2-amine), METHYL-MA (also known as 1-(4-methoxyphenyl)-N-methylpropan-2-amine), METHYL-MMDA-2 (also known as 1-(6-methoxy-1,3-benzodioxol-5-yl)-N-methylpropan-2-amine), MMDA (also known as 1-(7-methoxy-1,3-benzodioxol-5-yl)propan-2-amine), MMDA-2 (also known as 1-(6-methoxy-1,3-benzodioxol-5-yl)propan-2-amine), MMDA-3a (also known as 1-(4-methoxy-1,3-benzodioxol-5-yl)propan-2-amine), MMDA-3b (also known as 1-(7-methoxy-1,3-benzodioxol-4-yl)propan-2-amine), MME (also known as 1-(5-ethoxy-2,4-dimethoxyphenyl)propan-2-amine), MP (also known as 2-(3,4-dimethoxy-5-propoxyphenyl)ethanamine), MPM (also known as 1-(2,4-dimethoxy-5-propoxyphenyl)propan-2-amine), NBOMe-mescaline (also known as N-[(2-methoxyphenyl)methyl]-2-(3,4,5-trimethoxyphenyl)ethanamine), ORTHO-DOT (also known as 1-(4,5-dimethoxy-2-methylsulfanylphenyl)propan-2-amine), P (also known as 2-(3,5-dimethoxy-4-propoxyphenyl)ethanamine), PE (also known as 2-[3,5-dimethoxy-4-(2-phenylethoxy)phenyl]ethanamine), PEA (also known as 2-phenylethanamine), PROPYNYL (also known as 2-(3,5-dimethoxy-4-prop-2-ynoxyphenyl)ethanamine), psi-2C-T-4, psi-DOM (also known as 1-(2,6-dimethoxy-4-methylphenyl)propan-2-amine), SB (also known as 2-(3,5-diethoxy-4-methoxyphenyl)ethanamine), TA (also known as 1-(2,3,4,5-tetramethoxyphenyl)propan-2-amine), TB (also known as 2-(4-butylsulfanyl-3,5-dimethoxyphenyl)ethanamine), TCB-2 (also known as (3-bromo-2,5-dimethoxy-7-bicyclo[4.2.0]octa-1(6),2,4-trienyl)methanamine; hydrobromide), TMA (also known as 1-(3,4,5-trimethoxyphenyl)propan-2-amine), TMA-2 (also known as 1-(2,4,5-trimethoxyphenyl)propan-2-amine), TMA-3 (also known as 1-(2,3,4-trimethoxyphenyl)propan-2-amine), TMA-4 (also known as 1-(2,3,5-trimethoxyphenyl)propan-2-amine), TMA-5 (also known as 1-(2,3,6-trimethoxyphenyl)propan-2-amine), TMA-6 (also known as 1-(2,4,6-trimethoxyphenyl)propan-2-amine), TMPEA (also known as 2-(2,4,5-trimethoxyphenyl)ethanamine), TOMSO (also known as 1-(2-methoxy-4-methyl-5-methylsulfinylphenyl)propan-2-amine), TP (also known as 2-(3,5-dimethoxy-4-propylsulfanylphenyl)ethanamine), and TRIS (also known as 2-(3,4,5-triethoxyphenyl)ethanamine). For additional information regarding these compounds, see Shulgin, A. T., & Shulgin, A. (2016). Tihkal: The Continuation. Berkeley, CA: Transform Press.

In some embodiments, the serotonergic drug is chosen from alprazolam, amphetamine, aripiprazole, azapirone, a barbiturate, bromazepam, bupropion, buspirone, a cannabinoid, chlordiazepoxide, citalopram, clonazepam, clorazepate, dextromethorphan, diazepam, duloxetine, escitalopram, fluoxetine, flurazepam, fluvoxamine, lorazepam, lysergic acid diethylamide, lysergamide, 3,4-methylenedioxymethamphetamine, milnacipran, mirtazapine, naratriptan, paroxetine, pethidine, phenethylamine, psicaine, oxazepam, reboxetine, serenic, serotonin, sertraline, temazepam, tramadol, triazolam, a tryptamine, venlafaxine, vortioxetine, and/or derivatives and/or combinations thereof.

In some embodiments, the serotonergic drug is a tricyclic antidepressant (TCA), which can include amineptine, amitriptyline, amitriptylinoxide, amoxapine, butriptyline, clomipramine, desipramine, demexiptiline, dibenzepin, dimetacrine, doxepine, fluacizine, imipramine, imipraminoxide, iprinodole, lofepramine, maprotiline, melitracen, metapramine, nitroxazepine, nortripyline, noxiptiline, opipramol, protriptyline, propizepine, tianeptine, trimipramine, and quinupramine.

In some embodiments, the serotonergic drug is a selective serotonin reuptake inhibitor (SSRI), which can include citalopram, dapoxetine, escitalopram, fluoxetine, fluvoxamine, paroxetine, sertraline, and vilazodone.

In some embodiments, the serotonergic drug is a serotonin norepinephrine reuptake inhibitor (SNRI), which can include desvenlafaxine, duloxetine, levomilnacipran, venlafaxine, and desvenlafaxine.

A person of skill in the art would understand the terms tricyclic, SSRI, SNRI, and NRI are descriptive terms, wherein a particular compound could be described by one or more of the previous terms. Additionally, there may be neurotransmitter activity modulators contemplated by the current disclosure which may be described by other or additional terms (e.g., buproprion can be described as an atypical antidepressant).

As used herein, the term “dopaminergic drug” refers to a compound that binds, blocks, or otherwise influences activity at a dopamine receptor. In one embodiment, a dopaminergic drug is a dopamine transporter inhibitor. In one embodiment, a dopaminergic drug is a vesicular monoamine transporter inhibitor.

In one embodiment, a dopaminergic drug is chosen from amineptine, apomorphine, benzylpiperazine, bromocriptine, cabergoline, chlorpromazine, clozapine, dihydrexidine, domperidone, dopamine, fluphenazine, haloperidol, ketamine, loxapine, methamphetamine, olanzapine, pemoline, perphenazine, pergolide, phencyclidine, phenethylamine, phenmetrazine, pimozide, piribedil, a psychostimulant, reserpine, risperidone, ropinirole, tetrabenazine, thioridazine, or a combination thereof.

As used herein, the term “adrenergic drug” refers to a compound that binds, blocks, or otherwise influences activity at an adrenergic receptor. In one embodiment, an adrenergic drug binds to an adrenergic receptor. In one embodiment, an adrenergic drug indirectly affects an adrenergic receptor, e.g., via interactions affecting the reactivity of other molecules at the adrenergic receptor. In one embodiment, an adrenergic drug is an agonist, e.g., a compound activating an adrenergic receptor. In one embodiment, an adrenergic drug is an antagonist, e.g., a compound binding but not activating an adrenergic receptor, e.g., blocking a receptor. In one embodiment, an adrenergic drug is an effector molecule, e.g., a compound binding to an enzyme for allosteric regulation. In one embodiment, an adrenergic drug acts (either directly or indirectly) at more than one type of receptor (e.g., 5HT, dopamine, adrenergic, acetylcholine, etc.). In one embodiment, an adrenergic drug is an antidepressant. In one embodiment, an adrenergic drug is a norepinephrine transporter inhibitor (NRI). In one embodiment, an adrenergic drug is a vesicular monoamine transporter inhibitor.

In one embodiment, an adrenergic drug is chosen from adrenaline, agmatine, amoxapine, aptazapine, atomoxetine, bupropion, clonidine, doxepin, duloxetine, esmirtazpine, mianserin, mirabegron, mirtazapine, norepinephrine, phentolamine, phenylephrine, piperoxan, reserpine, ritodrine, setiptiline, tesofensine, timolol, trazodone, trimipramine, or xylazine.

In some embodiments, an adrenergic drug is considered to be a norepinephrine reuptake inhibitor (NRI). In some embodiments, an NRI can include bupropion or atomoxetine.

In some embodiments, the compositions of the present disclosure contain an anxiolytic drug. Non-limiting examples of anxiolytic drugs include alprazolam, an alpha blocker, an antihistamine, a barbiturate, a beta blocker, bromazepam, a carbamate, chlordiazepoxide, clonazepam, clorazepate, diazepam, flurazepam, lorazepam, an opioid, oxazepam, temazepam, or triazolam.

In some embodiments, the compositions of the present disclosure contain an antidepressant. Non-limiting examples of antidepressants include buproprion, citalopram, duloxetine, escitalopram, fluoxetine, fluvoxamine, imipramine, milnacipran, mirtazapine, paroxetine, reboxetine, sertraline, and venlafaxine.

### Combination Compositions

Also provided herein are orally administrable compositions (e.g., pharmaceutical compositions) comprising a therapeutically effective combination of cannabidiol and/or a derivative thereof, and/or psilocybin and/or a derivative thereof, and/or psilocin and/or a derivative thereof, and optionally mushroom blends, and various combinations thereof. The therapeutically effective combination of cannabidiol, and/or psilocybin, and/or psilocin, and optionally mushroom blends may be provided as a combination composition wherein each of the cannabidiol, and/or psilocybin, and optionally mushroom blends are provided as separate compositions in separate dosage forms, or may be provided as a combination composition wherein each of the cannabidiol, and/or psilocybin, and/or psilocin, and optionally mushroom blends are provided in a single composition, in a single dosage form or multiple dosage forms containing divided doses of the composition. Thus, for example, a combination composition may include one or more capsules containing mushroom blends, one or more capsules containing psilocybin and/or psilocin, and one or more capsules containing cannabidiol, or may include one or more capsules containing each of mushroom blends, psilocybin and/or psilocin, and cannabidiol. Alternatively, two or more of a mushroom blend, psilocybin and/or psilocin, and cannabidiol may be provided in the same composition, in a single dosage forms or multiple dosage forms containing divided doses. Thus, for example, a combination composition may include one or more capsules containing mushroom blends and cannabidiol and one or more capsules containing psilocybin and/or psilocin only, or one or more capsules containing mushroom blends and psilocybin, and one or more capsules containing cannabidiol only, or one or more capsules containing mushroom blends and cannabidiol, and one or more capsules containing mushroom blends, wherein the psilocybin may be contained in either type of capsule.

In any of these embodiments, the mushroom blends of the combination may be provided in a single composition, in a single dosage form or multiple dosage forms containing divided doses, or may be provided in separate compositions, wherein each composition may comprise different, overlapping, or the same mushroom varieties, in different, overlapping or same amounts. In any of these embodiments, one or more or all of the compositions may optionally further comprise a pharmaceutically acceptable carrier or excipient suitable for oral administration. In some embodiments, the maintenance dose can be a solid dosage form or a liquid.

The compositions optionally may further include a composition comprising a loading dose of psilocybin as described herein, in a single dosage form or multiple dosage forms containing divided doses. In some embodiments, a loading dose can be a solid dosage form or a liquid.

In some instances, a composition can include psilocybin or derivatives thereof, in combination with a neurotransmitter activity modulator. In some embodiments the neurotransmitter activity modulator is a serotonergic neurotransmitter activity modulator. In some embodiments, the neurotransmitter activity modulator is a tricyclic antidepressant. In some embodiments the neurotransmitter activity modulator is an SSRI. In some embodiments the neurotransmitter activity modulator is an SNRI. In some embodiments, the neurotransmitter activity modulator is a norepinephrine reuptake inhibitor (NRI). As with the combination of psilocybin and cannabidiol, and optionally a mushroom blend, the combination composition can comprise a single dosage form or multiple dosage forms containing divided doses of the composition. The combination composition can comprise separate dosages or dosage forms for the different combination components. In some instances, a composition can include adding one of identified psilocybin, and/or CBD, and/or neurotransmitter activity modulator, and/or mushroom blend compositions to another identified composition (e.g., psilocybin, and/or CBD, and/or neurotransmitter activity modulator, and/or mushroom blend). For example, a compsosition of the disclosure can include adding psilocybin to any of the identified neurotransmitter activity modulators identified herein (e.g., adding psilocybin to a tricyclic antidepressant, an SSRI, and SNRI, and/or an NRI).

The compositions described herein can be manufactured by conventional methods, such as those including mixing, dissolving, granulating, levigating, emulsifying, encapsulating, and/or lyophilization processes. The compositions can be formulated in accordance with conventional practices, optionally with one or more pharmaceutically acceptable carriers or excipients suitable for oral administration.

Therapeutic combination products with complementary, additive, or synergistic mechanisms of action can provide greater efficacy while reducing adverse effect profiles. Combination products are frequently used in the therapeutic management of multiple and complex diseases/conditions including but not limited to methods for treating traumatic brain injury (TBI). Other neurological conditions (e.g., anxiety, depression, suicide ideation, stress, post-concussion syndrome, post-traumatic headache, and/or progressive headache) or symptoms thereof, are also treatable with the methods described herein. The novel integrated combination of psilocybin and/or psilocin, and cannabidiol (CBD) provides therapeutic synergistic benefits based on their differences in molecular structure and complementary mechanisms of action on the serotonergic system that yield synergistic physiologic neural health benefits.

Psilocybin (0-phosphoryl-4-hydroxy-N,N-dimethyltryptamine) and its active metabolite psilocin (4-hydroxy-N,N-dimethyltryptamine) are tryptamine/indolamine hallucinogens that are chemically and structurally related to serotonin. Psilocin is the active molecule that produces the pharmacologic effects of a selective agonist of serotonin (5-HT) family of receptors, which includes the 5-HT1A, 5-HT2A, 5-HT2B, and 5-HT2C receptor subtypes. Preclinical studies have shown that 5-HT2A receptor activation in the cortical and subcortical structures is one of the primary mechanisms that results in pharmacologic effects. Psilocybin also significantly decreases 11C-raclopride binding potential bilaterally in the caudate nucleus and putamen, which results in a reciprocal increase in endogenous dopamine. Dual actions from the presynaptic and postsynaptic 5-HT2A receptor activation by psilocin forms a cyclic feedback process for 5-HT2A receptor activation of glutamate effects in the central nervous system, leading to a complex cortical-thalamic neurocircuitry. These mechanisms along with a potential for increasing neurogenesis and neural plasticity and improving cerebral blood flow contribute to the established therapeutic benefits of psilocybin to overall central nervous system (CNS) health and in management of disease (e.g., depression, anxiety, and traumatic brain injury).

Cannabidiol (2-[(6R)-3-methyl-6-prop-1-en-2-ylcyclohex-2-en-1-yl]-5-pentylbenzene-1,3-diol) is a phytocannabinoid derived from Cannabis species, which is devoid of psychoactive activity, with analgesic, anti-inflammatory, and neuroprotective, and anxiolytic activities. Upon administration, cannabidiol (CBD) exerts activity through various mechanisms. Cannabidiol binds to CB1 receptor and changes the binding site through negative allosteric modulation which is thought to provide its anxiolytic properties (e.g., mitigation of the anxiety associated with THC consumption). Cannabidiol can act as a positive modulator of 5HT1A-mediated neurotransmission or as an agonist at transient receptor potential cation channel subfamily V member 1 (TRPV1) and peroxisome proliferator-activated receptor gamma (PPARγ) receptors.

CBD also increases the natural cannabinoid anandamide levels by blocking reuptake and inhibiting the enzymatic breakdown of anandamide. Cannabidiol has been shown to promote a complex set of changes in crucial intracellular pathways such as mammalian target of rapamycin (mTOR), autophagy, and glycogen synthase kinase 3 beta (GSK30) resulting in neuroprotection, decreased inflammation and increased neuroplasticity which suggests benefits for overall CNS health and in management of neuropsychiatric disease. Additionally, cannabidiol demonstrates positive effects on attenuating psychotic, anxiety, and depressive-like behaviors.

Through complementary mechanisms of action, the integrated combination product(s) of psilocybin and CBD, and optionally with a mushroom blend, results in therapeutic synergistic benefits by increasing cerebral blood flow, improving neuronal crosstalk, and establishing new neural connections that directly affect serotonergic pathways and provide beneficial neuroprotective and anti-inflammatory effects.

Additionally, serotonergic activity modulators, for example tricyclic antidepressants, SSRIs, and SNRIs can have synergistic and beneficial activities when taken with (e.g., in a single composition or in a schedule or regimen) psilocybin and/or psilocin. An example of a synergistic effect is a greater than expected antidepressant effect when a serotonergic activity modulator is administered in conjunction with or after administration of a psilocybin loading dose. In some embodiments, administration of a psilocybin loading dose along with a serotonergic activity modulator increases the antidepressant effect as compared to psilocybin and/or psilocin alone, the serotonergic activity modulator alone, and is more than an additive effect of the psilocybin and serotonergic activity modulator together. In some embodiments, administration of a psilocybin loading dose allows for less serotonergic activity modulator to be administered while achieving the same antidepressant effect associated with a higher dosage of serotonergic activity modulator. This can be especially advantageous for some serotonergic activity modulators having a significant side effect profile, including cardiovascular and neurological toxicity. For example, the tricyclic antidepressant class of drugs can be associated with severe overdose symptoms, including fatal drug poisoning. Therefore, at least one advantage of the compositions and methods described herein is the ability to elicit a particular antidepressant effect while administering less tricyclic antidepressant than if the tricyclic antidepressant were administered alone. Thus, an advantage of the compositions and methods described herein can include decreasing adverse risk profiles associated with serotonergic activity modulators.

Norepinephrine reuptake inhibitors (NRIs) can have synergistic and beneficial activities when taken with (e.g., in a single composition or in a schedule or regimen) psilocybin and/or psilocin. As psilocybin activates serotonergic receptors, effecting the serotonergic system, pairing psilocybin with a NRI creating a novel SNRI composition. This novel SNRI composition can have a beneficial adverse effect profile as compared to known SNRI compounds. In some embodiments, the novel composition is more efficacious at treating a symptom of migraine (e.g., anxiety or depression) as compared to known SNRI compounds at a particular dosage. In some embodiments, administration of psilocybin, as a loading dose or a maintenance dose, allows for less NRI to be administered while achieving the same antidepressant effect associated with a higher dosage of NRI or SNRI. Therefore, an advantage of the compositions and methods described herein can include decreasing risk profiles associated with NRIs or SNRIs.

### Additives and Other Components

The compositions of the present disclosure can include various additives or other components.

The compositions of the present disclosure can comprise turmeric. In some embodiments, the composition comprises about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt % 6 wt %, 7 wt % 8 wt %, 9 wt %, 10 wt %, 20 wt %, 30 wt %, 40 wt %, 50 wt % 60 wt % 70 wt %, or 80 wt % turmeric. In some embodiments, the composition comprises turmeric in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt %, 9 wt %, 10 wt %, 20 wt %, 30 wt %, 40 wt %, 50 wt %, 60 wt %, 70 wt %, or 80 wt %. In various embodiments, the compositions of the present disclosure comprise about or at least about 10 mg to about 150 mg turmeric, 20 mg to about 150 mg, 30 mg to about 150 mg, 40 mg to about 150 mg, 50 mg to about 150 mg, 60 mg to about 150 mg, 70 mg to about 150 mg, 80 mg to about 150 mg, 90 mg to about 150 mg, 100 mg to about 150 mg, or about 125 mg to about 150 mg turmeric. In various embodiments, the compositions of the present disclosure comprise no more than about 0.01 mg to about 10 mg, 0.01 mg to about 20 mg, 0.01 mg to about 30 mg, 0.01 mg to about 40 mg, 0.01 mg to about 50 mg, 0.01 mg to about 60 mg, 0.01 mg to about 70 mg, 0.01 mg to about 80 mg, 0.01 mg to about 90 mg, 0.01 mg to about 100 mg, or 0.01 mg to about 150 mg turmeric.

The compositions of the present disclosure can comprise niacin. In various embodiments, the niacin comprises about or at least about 0.5 mg to about 10 mg, 1 mg to about 10 mg, 2 mg to about 10 mg, 3 mg to about 10 mg, 4 mg to about 10 mg, 5 mg to about 10 mg, 6 mg to about 10 mg, 7 mg to about 10 mg, 8 mg to about 10 mg, or about 9 mg to about 10 mg niacin. In various embodiments, the compositions comprises no more than about 0.01 mg to about 0.5 mg, about 0.01 mg to about 1 mg, about 0.01 mg to about 2 mg, about 0.01 mg to about 3 mg, about 0.01 mg to about 4 mg, about 0.01 mg to about 5 mg, about 0.01 mg to about 6 mg, about 0.01 mg to about 7 mg, about 0.01 mg to about 8 mg, or about 0.01 mg to about 10 mg niacin.

The compositions of the present disclosure can comprise piperine. In some embodiments, the composition comprises about or at least about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt % 5 wt %, 6 wt % 7 wt % 8 wt %, 9 wt %, 10 wt %, 20 wt %, 30 wt %, 40 wt %, 50 wt %, 60 wt %, 70 wt %, or 80 wt % turmeric. In some embodiments, the composition comprises piperine in an amount of no more than about 0.01 wt %, 0.02 wt %, 0.03 wt %, 0.04 wt %, 0.05 wt %, 0.1 wt %, 0.2 wt %, 0.3 wt %, 0.4 wt %, 0.5 wt %, 0.75 wt %, 1 wt %, 2 wt %, 3 wt %, 4 wt %, 5 wt %, 6 wt %, 7 wt %, 8 wt %, 9 wt %, 10 wt %, 20 wt %, 30 wt %, 40 wt %, 50 wt %, 60 wt %, 70 wt %, or 80 wt %.

The compositions of the present disclosure can include terpenes (or terpenoids). Terpenes are volatile organic compounds found in many plants. Terpenes can correct or enhance the effect of a compound (e.g., a cannabinoid) so that less of the compound is necessary to achieve a therapeutic effect. In some embodiments, terpenes can reduce a psychoactive effect of a compound. Terpenes are typically derived biosynthetically from units of isoprene.

In some embodiments, the compositions of the disclosure comprise one or more of the following non-limiting examples of terpenes: acetanisole, acetyl cedrene, anethole, anisole, benzaldehyde, bornyl acetate, borneol, cadinene, cafestol, caffeic acid, camphene, camphor, capsaicin, carene, carotene, carvacrol, carvone, alpha-caryophyllene, beta-caryophyllene, caryophyllene oxide, cedrene, cedrene epoxide, cecanal, cedrol, cembrene, cinnamaldehyde, cinnamic acid, citronellal, citronellol, cymene, eicosane, elemene, estragole, ethyl acetate, ethyl cinnamate, ethyl maltol, eucalyptol/1,8-cineole, eudesmol, eugenol, euphol, farnesene, farnesol, fenchone, geraniol, geranyl acetate, guaia-1 (10), 11-diene, guaiacol, guaiol, guaiene, gurjunene, herniarin, hexanaldehyde, hexanoic acid, humulene, ionone, ipsdienol, isoamyl acetate, isoamyl alcohol, isoamyl formate, isoborneol, isomyrcenol, isoprene, isopulegol, isovaleric acid, lavandulol, limonene, gamma-linolenic acid, linalool, longifolene, lycopene, menthol, methyl butyrate, 3-mercapto-2-methylpentanal, beta-mercaptoethanol, mercaptoacetic acid, methyl salicylate, methylbutenol, methyl-2-methylvalerate, methyl thiobutyrate, beta-myrcene, gamma-muurolene, nepetalactone, nerol, nerolidol, neryl acetate, nonanaldehyde, nonanoic acid, ocimene, octanal, octanoic acid, pentyl butyrate, phellandrene, phenylacetaldehyde, phenylacetic acid, phenylethanethiol, phytol, pinene, propanethiol, pristimerin, pulegone, retinol, rutin, sabinene, squalene, taxadiene, terpineol, terpine-4-ol, terpinolene, thujone, thymol, umbelliferone, undecanal, verdoxan, and vanillin.

In some embodiments, the compositions of the present disclosure contain a flavoring agent. In some embodiments, the flavoring agent comprises vanillin and/or peppermint. In some embodiments, the flavoring agent is a carbohydrate (e.g., a sugar).

In some embodiments, the compositions can comprise a pharmaceutically acceptable carrier, such as diluents, fillers, salts, buffers, stabilizers, solubilizers, and the like. In some embodiments, the compositions can comprise an anti-caking and/or anti-clumping agent, e.g., a desiccant. The compositions may contain pharmaceutically acceptable excipients for modifying conditions such as pH, osmolarity, taste (e.g., a flavoring agent), viscosity, sterility, lipophilicity, and/or solubility. The choice of diluents, carriers, or excipients will depend on the desired dosage form, which may in turn be dependent on the intended route of administration to a subject.

In some embodiments, the compositions can comprise an antioxidant. The antioxidant can be selected from ascorbic acid, lycopene, tocopherol, melatonin, retinol, astaxanthin, lutein, apigenin, carnosine, selenium, zinc, curcumin, and/or a salt or derivative thereof.

A composition may comprise small amounts of liquid that are negligible in the final measurement of a sample. In one example, it is acceptable for a composition of this disclosure to comprise as much as 1% water as measured by mass percent.

Solid carriers and excipients are generally known in the art and include, as non-limiting examples, magnesium carbonate, magnesium stearate, talc, sugar, or lactose.

### Dosage Forms

The compositions of the present disclosure may be formulated as pharmaceutical or nutraceutical dosage forms. The formulations can be used in the treatment of a neurological condition (e.g., anxiety, depression, suicide ideation, stress, traumatic brain injury, post-concussion syndrome, post-traumatic headache, and/or progressive headache). In some embodiments, the compositions of the present disclosure are formulated as dosage forms for administration to a subject. The dosage form can be a solid dosage form (e.g., tablets, capsules, powders, dispersible granules, cachets, and suppositories, including sustained release and delayed release formulations. Powders and tablets in various embodiments may comprise from about 0.1% to about 70% active ingredient. Tablets, powders, cachets, fast-dissolving films, liquids, extracts, tinctures, and capsules are all non-limiting examples of suitable dosage forms for oral administration. In some embodiments, the dosage form is a cream. Standard texts, such as “Remington's Pharmaceutical Science”, 17th edition, 1985, incorporated herein by reference, may be consulted to prepare suitable dosage forms, without undue experimentation.

In some embodiments, the compositions of the present disclosure may be combined with a food to be consumed by a subject. As a non-limiting example, the dosage forms of the present disclosure may include a solid (e.g., a powder) or liquid (e.g., a tincture, solution, or extraction) combined with a solid and/or liquid food for consumption by a subject. In some embodiments, the liquid food may be a beverage (e.g., water or a smoothie). In some embodiments, the dosage form may be a foodstuff (e.g., a nutritional powder). In some embodiments, the compositions of the present disclosure may be used as a food ingredient.

In some embodiments, the dosage form may be a sachet. The sachet may in some embodiments be dissolvable in water. Liquid dosage forms may be administered orally or by intravenous, intracerebral, intraperitoneal, parenteral, or intramuscular injection, or infusion. Sterile injectable formulations may comprise a sterile solution or suspension of an active agent in a non-toxic, pharmaceutically acceptable diluent or solvent. Dosage forms, including liquid dosage forms, also include solutions, sprays, or other suitable dosage forms for intranasal, buccal, or sublingual administration. Suitable liquid dosage forms include solutions, suspensions, and emulsions.

In some embodiments, the pharmaceutical formulation is a parenteral dosage form. In some embodiments, the dosage form is suitable for transdermal administration, including creams, lotions, aerosols, and/or emulsions. These dosage forms may be included in transdermal patches of a matrix or reservoir type. In some embodiments, the pharmaceutical formulation is an oral dosage form. In some embodiments, the pharmaceutical composition comprises a tablet. In some embodiments, the pharmaceutical composition comprises a capsule. In some embodiments, the pharmaceutical composition comprises a dry powder. In some embodiments, the pharmaceutical composition comprises a solution. In some embodiments, more than one dosage form is administered to the subject at substantially the same time. In some embodiments, the subject may be administered the entire therapeutic dose in one tablet or capsule. In some embodiments, the therapeutic dose may be split among multiple tablets or capsules. For example, for a dose of 25 mg, the subject may be administered 5 tablets or capsules each comprising 5 mg of psilocybin. Alternatively, for a dose of 30 mg, the subject may be administered 3 tablets or capsules each comprising 10 mg of psilocybin.

In some embodiments, an oral dosage form comprises a functional filler. The functional filler may be a silicified filler, such as, but not limited to silicified microcrystalline cellulose (SMCC). In some embodiments, the oral dosage form comprises high compatibility grades of SMCC with a particle size range of from about 45 to 150 microns. A mixture of two functional fillers having different particle size ranges may be used with the weight percentages of the two favoring the larger sized particles.

In some embodiments, the silicified microcrystalline filler may comprise a first filler, having a particle size range of from about 45 to 80 microns in an amount of up to 30%, up to 20%, up to 15%, or less by weight of filler, and a second filler, having a particle size range of from about 90 to 150 microns, in an amount of up to 70%, up to 80%, up to 85%, or more, by weight of filler. In some embodiments, a mushroom or mushroom blend can be used as a filler.

The formulation may further comprise or consist essentially of a disintegrant, including without limitation sodium starch glycolate; a glidant, including without limitation colloidal silicon dioxide; and a lubricant, including without limitation sodium stearyl fumarate. In some embodiments, the oral dosage form may comprise a disintegrant such as sodium starch glycolate.

In some embodiments, one or more compositions of the present disclosure are administered to a subject as separate dosage forms. In some embodiments, one or more components of a composition are administered as separate dosage forms. In some embodiments, the dosage form comprises one, two, three, or more pills. In some embodiments, the dosage form comprises different compositions each in a separate form; for example, a pill in combination with a liquid solution or two pills in combination with a liquid solution. In some embodiments, the dosage form comprises a first composition in a first dosage form, a second composition in a second dosage form, a third composition in a third dosage form, and various combinations thereof.

For example, a dosage form may consist of four capsules, wherein each of three capsules may consist of cannabidiol, Hericium erinaceus, Laricifomes officinalis, Cordyceps sp., Ganoderma lingzhi, Trametes versicolor, and Inonotus obliquus; and the remaining capsule may consist of Inonotus obliquus, Agaricus subrufescens, Laricifomes officinalis, Trametes versicolor, Ganoderma lingzhi, Schizophyllum commune, Hericium erinaceus, Wolfiporia extensa, Cordyceps sp., Fomes fomentarius, Pleurotus ostreatus, Phellinus linteus, Grifola frondosa, Lentinula edodes, or various combinations thereof. In some embodiments, the capsules further comprise a fungus of the genus Psilocybe (e.g., Psilocybe cubensis, P. azurescens, P. semilanceata, or P. cyanescens). In some embodiments, the capsules further comprise cannabidiol. Thus, the compositions disclosed herein may be orally administered in one or more capsules, such as 1, 2, 3, 4, 5, or more capsules.

In some embodiments, the dosage form is suitable for inhalation. The dosage form may contain solutions and solids in powder form. In some embodiments, the dosage form may comprise a composition combined with a pharmaceutically acceptable carrier, such as an inert compressed gas.

In some embodiments, the composition is suitable for transdermal administration, including creams, lotions, aerosols, and/or emulsions. These compositions may be included in transdermal patches of a matrix or reservoir type.

A cannabinoid may be administered in any suitable dosage form, such as any oral, or transmucosal dosage form (1-10 mg approximately), such as may be selected from tinctures (1-50 mg), fast-dissolving tablets, tablets (1-50 mg), solutions (1-50 mg), gums (1-50 mg), gummies (1-50 mg), and for application to a site in the oral cavity. For example, synthetic cannabidiol or purified cannabidiol may be administered in an oral dosage form, such as in an oral capsule. A cannabidiol may be in the form of dry sift kief. The cannabidiol may be in the form of dry sift kief that has been decarboxylated to an activated form, and then subsequently freeze-dried. The freeze-dried composition may then be encapsulated. Thus, the cannabidiol may be in the form of encapsulated, freeze-dried, decarboxylated dry sift kief. Cannabidiol in the form of dry sift kief is understood to be full-spectrum cannabidiol.

### Methods of Treating TBI Using Compositions Described Herein

Described herein are methods for treating a neurological condition (e.g., anxiety, depression, suicide ideation, stress, traumatic brain injury, post-concussion syndrome, post-traumatic headache, progressive headache, and/or acute or chronic ischemic events) with a composition comprising a cannabinoid and/or a derivative thereof, and/or psilocybin and/or a derivative thereof, and/or CBD, and optionally a mushroom blend, and various combinations thereof. In some embodiments, the methods for treating a neurological condition or symptom thereof include administering a composition comprising psilocybin, or a derivative thereof, in combination with a neurotransmitter activity modulator (e.g., a tricyclic, SNRI, SSRI, or NRI antidepressant). The present disclosure provides methods of treating a neurological condition or symptoms thereof (e.g., anxiety and/or depression) which comprise administering a therapeutically effective amount of a composition of the present disclosure. The method includes the step of administering to a subject (e.g. a mammal) a therapeutic amount of a composition of the present disclosure sufficient to treat the condition and/or a symptom thereof, under conditions such that the condition or symptom thereof is treated. The methods herein include administering to the subject (including a subject identified as in need of such treatment) an effective amount of a composition described herein, or a composition described herein to produce such effect.

In some embodiments the TBI is associated with other psychological or neurological conditions. As used herein, neurological conditions associated with TBI include, but are not limited to, anxiety, depression, suicide ideation, stress, traumatic brain injury, post-concussion syndrome, post-traumatic stress disorder, post-traumatic headache, and progressive headache. The compositions and methods described herein also may be useful for treating conditions associated with or resulting from traumatic brain injury post-concussion syndrome, post-traumatic headache, or progressive headache including, as non-limiting examples, headaches, feelings of dizziness, nausea and/or vomiting, noise sensitivity, being easily upset by loud noise, sleep disturbance, fatigue or tiring more easily, irritability, insomnia, depression, suicide ideation, nervousness, frustration or impatience, forgetfulness or poor memory, poor concentration, taking longer to think, blurred vision, light sensitivity, double vision, restlessness, anxiety, insomnia, loss of concentration and memory, ringing in the ears, blurry vision, decrease in taste and/or smell, and various combinations thereof.

In one embodiment, the disclosure provides methods of monitoring treatment progress. The method includes the step of determining a level of a diagnostic measurement in a subject suffering from or susceptible to a condition or symptoms thereof associated with a traumatic brain injury (e.g., post-concussion syndrome, post-traumatic headache, or progressive headache), in which the subject has been administered a therapeutic amount of a compound described herein sufficient to treat the disease or symptoms thereof. The level of diagnostic measurement determined in the method can be compared to known levels of the diagnostic measurement in either healthy normal controls or in other afflicted patients to establish the subject's condition status. In preferred embodiments, a second level of the diagnostic measurement in the subject is determined at a time point later than the determination of the first level, and the two levels are compared to monitor the course of disease or the efficacy of the therapy. In certain preferred embodiments, a pre-treatment level of diagnostic marker in the subject is determined prior to beginning treatment according to the methods of the present disclosure; this pre-treatment level of the diagnostic measurement can then be compared to the level of diagnostic measurement in the subject after the treatment commences, to determine the efficacy of the treatment.

In various embodiments, provided herein are methods of treating a neurological condition (e.g., TBI), or one or more symptoms thereof in a patient in need thereof, comprising orally administering to the patient a therapeutically effective composition containing psilocybin and/or a derivative thereof, cannabinoid and/or a derivative thereof, and optionally a mushroom blend or various combinations thereof. In some embodiments, the methods for treating TBI or a symptom thereof include administering a composition comprising psilocybin, or a derivative thereof, in combination with a neurotransmitter activity modulator (e.g., a tricyclic, SSRI, SNRI, or NRI antidepressant). As discussed in more detail above, the mushroom blend in various embodiments comprises one or more dried mushrooms from one or more of the following genuses: Inonotus (e.g., Chaga), Agaricus (e.g., Blazei), Laricifomes (e.g., Agarikon), Trametes (e.g., Turkey Tail), Ganoderma (e.g., Reishi), Schizophyllum (e.g., Suehirotake), Hericium (e.g., Lion's Mane), Wolfiporia (e.g., Poria), Cordyceps (e.g., Cordyceps; e.g., Cordyceps militaris), Fomes (e.g., True Tinder Polypore), Pleurotus (e.g., Oyster Mushroom), Phellinus (e.g., Mesima), Grifola (e.g., Maitake), and Lentinula (e.g., Shiitake), and various combinations thereof.

The methods may be performed with any of the psilocybin, cannabidiol, neurotransmitter activity modulator, and optionally mushroom blend compositions described above.

An effective combination of psilocybin and/or a derivative thereof, cannabinoid and/or a derivative thereof, and/or a neurotransmitter activity modulator or derivative thereof, and optionally a mushroom blend or various combinations thereof may be administered in a single dosage form or in multiple dosage forms, which may be administered concurrently, at substantially the same time, or at different times over the course of a day, week, or month. In some embodiments, an agent described herein is administered in divided doses over the course of a day, such as in two, three, four, or more, divided doses. In some embodiments, the composition is administered in a single dose (in single or multiple dosage forms) once a day, optionally on an empty stomach or on a full stomach. If the composition is administered in multiple dosage forms, each of the mushroom blend, psilocybin or a derivative thereof, and cannabidiol or a derivative thereof, and/or neurotransmitter activity modulator may be provided in one or more separate dosage forms (e.g., one or more capsules containing mushroom blend, one or more capsules containing psilocybin, and one or more capsules containing cannabidiol), or a single dosage form may comprise two or more of mushroom blend, psilocybin, a neurotransmitter activity modulator, and cannabidiol (e.g., one or more capsules containing mushroom blend and cannabidiol and one or more capsules containing psilocybin only, one or more capsules containing mushroom blend and psilocybin and one or more capsules containing cannabidiol only, or one or more capsules containing mushroom blend and cannabidiol and one or more capsules containing mushroom blend only, wherein the psilocybin may be contained in either type of capsule). In any of these embodiments, the mushroom blend may be provided in a single dosage form or multiple dosage forms, wherein each dosage form may comprise different, overlapping, or the same mushroom varieties, in different, overlapping or same amounts. In some embodiments, the administration of each of the following components: psilocybin, cannabinoid, a neurotransmitter activity modulator, and optionally a mushroom blend occurs within minutes, hours, or days of the administration of the other components.

### Loading Dose(s)

In some embodiments, the subject is administered a loading dose of psilocybin or a derivative thereof either before or after receiving a maintenance dose of psilocybin or a derivative thereof. The loading dose composition or dosage form of psilocybin can comprise a higher concentration of psilocybin or a derivative thereof than the maintenance dose.

The loading dose of psilocybin or a derivative thereof may include psilocybin or a derivative thereof in an amount of from about 1 mg to about 100 mg. In some embodiments, the psilocybin is synthetic psilocybin. In some embodiments, the psilocybin is crystalline psilocybin.

In some embodiments, the psilocybin is amorphous psilocybin. In some embodiments, the psilocybin is deuterated psilocybin. This includes about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 mg, and any amounts therebetween.

The loading dose of synthetic psilocybin or a derivative thereof may include psilocybin or a derivative thereof in an amount from about 1 mg to about 100 mg, about 1 mg to about 90 mg, about 1 mg to about 80 mg, about 1 mg to about 70 mg, about 1 mg to about 60 mg, about 1 mg to about 50 mg, about 1 mg to about 45 mg, about 1 mg to about 40 mg, about 1 mg to about 35 mg, about 1 mg to about 34 mg, about 1 mg to about 33 mg, about 1 mg to about 32 mg, about 1 mg to about 31 mg, about 1 mg to about 30 mg, about 1 mg to about 29 mg, about 1 mg to about 28 mg, about 1 mg to about 27 mg, about 1 mg to about 26 mg, about 1 mg to about 25 mg, about 1 mg to about 24 mg, about 1 mg to about 23 mg, about 1 mg to about 22 mg, about 1 mg to about 21 mg, about 1 mg to about 20 mg, about 1 mg to about 19 mg, about 1 mg to about 18 mg, about 1 mg to about 15 mg, about 1 mg to about 10 mg, about 1 mg to about 5 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 45 mg, about 10 mg to about 40 mg, about 10 mg to about 35 mg, about 10 mg to about 34 mg, about 10 mg to about 33 mg, about 10 mg to about 32 mg, about 10 mg to about 31 mg, about 10 mg to about 30 mg, about 10 mg to about 29 mg, about 10 mg to about 28 mg, about 10 mg to about 27 mg, about 10 mg to about 26 mg, about 10 mg to about 25 mg, about 10 mg to about 24 mg, about 10 mg to about 23 mg, about 10 mg to about 22 mg, about 10 mg to about 21 mg, about 10 mg to about 20 mg, about 10 mg to about 19 mg, about 10 mg to about 18 mg, about 10 mg to about 15 mg, about 15 mg to about 90 mg, about 15 mg to about 80 mg, about 15 mg to about 70 mg, about 15 mg to about 60 mg, about 15 mg to about 50 mg, about 15 mg to about 45 mg, about 15 mg to about 40 mg, about 15 mg to about 35 mg, about 15 mg to about 34 mg, about 15 mg to about 33 mg, about 15 mg to about 32 mg, about 15 mg to about 31 mg, about 15 mg to about 30 mg, about 20 mg to about 35 mg, about 21 mg to about 35 mg, about 22 mg to about 35 mg, about 22 mg to about 35 mg, about 23 mg to about 35 mg, about 24 mg to about 35 mg, about 25 mg to about 35 mg, about 20 mg to about 34 mg, about 21 mg to about 34 mg, about 22 mg to about 34 mg, about 23 mg to about 34 mg, about 23 mg to about 33 mg, about 23 mg to about 32 mg, about 23 mg to about 31 mg, about 24 mg to about 35 mg, about 24 mg to about 34 mg, about 24 mg to about 33 mg, about 24 mg to about 32 mg, about 24 mg to about 31 mg, about 24 mg to about 30 mg, about 25 mg to about 35 mg, about 25 mg to about 34 mg, about 25 mg to about 33 mg, about 25 mg to about 32 mg, about 25 mg to about 31 mg, or about 25 mg to about 30 mg psilocybin.

In some embodiments, the loading dose of synthetic psilocybin or derivative thereof or psilocin or derivative thereof, can be calculated based on the subject's body weight. In some embodiments, the loading dose of synthetic psilocybin comprises about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, or about 1.0 mg/kg of the subject's body weight. In some embodiments, the loading dose of synthetic psilocybin or a derivative thereof, or psilocin or a derivative thereof can comprise about 0.1 mg/kg to about 1 mg/kg, about 0.2 mg/kg to about 1 mg/kg, about 0.3 mg/kg to about 1 mg/kg, about 0.4 mg/kg to about 1 mg/kg, about 0.1 mg/kg to about 0.9 mg/kg, about 0.1 mg/kg to about 0.8 mg/kg, about 0.1 mg/kg to about 0.7 mg/kg, about 0.1 mg/kg to about 0.6 mg/kg, about 0.1 mg/kg to about 0.5 mg/kg, about 0.1 mg/kg to about 0.4 mg/kg, about 0.2 mg/kg to about 0.8 mg/kg, about 0.3 mg/kg to about 0.7 mg/kg, about 0.3 mg/kg to about 0.6 mg/kg, about 0.3 mg/kg to about 0.5 mg/kg, about 0.3 mg/kg to about 0.45 mg/kg, or about 0.3 mg/kg to about 0.4 mg/kg of the subject's body weight.

The loading dose of natural psilocybin may include from about 0.5 g to about 6 g of dried mushrooms, such as Psilocybe cubensis, or dried mushroom blend. This includes about 0.5 g, about 0.6 g, about 0.7 g, about 0.8 g, about 0.9 g, about 1.0 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, about 5.0 g, about 5.2 g, about 5.4 g, about 5.6 g, about 5.8 g, about 6.0 g. The loading dose may include psilocybin or a derivative thereof in an amount from about 0.5 g mg to about 6 g, about 1 g to about 6 g, about 1 g to about 5 g, about 1 g to about 4 g, about 1 g to about 3 g, about 1 g to about 2 g, about 2 g to about 6 g, about 2 g to about 5 g, about 2 g to about 4 g, about 2 g to about 3 g, about 3 g to about 6 g, about 3 g to about 5 g, about 3 g to about 4 g, about 4 g to about 6 g, about 4 g to about 5 g. In some embodiments, the loading dose of psilocybin or derivative thereof or psilocin or derivative thereof is about 5 g of dried mushrooms or a dried mushroom blend. In some embodiments the dried mushrooms or dried mushroom blend includes Psilocybe of Psilocybe cubensis.

The loading dose of natural psilocybin may include a dried mushroom, such as Psilocybe cubensis, or dried mushroom blend, which can be calculated based on the subject's body weight. In some embodiments, the loading dose comprises about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, or about 0.2 mg/kg of the subject's body weight. In some embodiments the loading dose psilocybin or a derivative thereof, or psilocin or a derivative thereof is in the form of a dried mushroom or dried mushroom blend, wherein the loading dose comprises about 0.01 mg/kg to about 0.2 mg/kg, about 0.02 mg/kg to about 0.2 mg/kg, about 0.03 mg/kg to about 0.2 mg/kg, about 0.04 mg/kg to about 0.2 mg/kg, about 0.05 mg/kg to about 0.2 mg/kg, about 0.06 mg/kg to about 0.2 mg/kg, about 0.07 mg/kg to about 0.2 mg/kg, about 0.08 mg/kg to about 0.2 mg/kg, about 0.09 mg/kg to about 0.2 mg/kg, about 0.07 mg/kg to about 0.1 mg/kg, about 0.06 mg/kg to about 0.11 mg/kg, about 0.05 mg/kg to about 0.13 mg/kg, about 0.4 mg/kg to about 0.13 mg/kg, about 0.4 mg/kg to about 0.14 mg/kg, about 0.4 mg/kg to about 0.14 mg/kg of the subject's body weight.

In some embodiments, the loading dose is administered in conjunction with a psychological support session. In some embodiments, the loading dose is not administered in conjunction with a psychological support session. This could be because the loading dose selected may be low enough to not alter the subject's state of consciousness.

### Maintenance Dose(s)

In some embodiments, the subject is administered a maintenance dose either before or after receiving a loading dose. In some embodiments, the subject is administered a maintenance dose in the absence of a loading dose of psilocybin. In some embodiments, a maintenance dose includes psilocybin and/or psilocin or a derivative thereof. The maintenance dose can comprise a lower concentration of psilocybin or a derivative thereof than the loading dose. In some embodiments, the maintenance dose includes a cannabinoid, such as cannabidiol (CBD), optionally with a mushroom blend. In some embodiments, the maintenance dose includes a neurotransmitter activity modulator (e.g., a tricyclic, SNRI, SSRI, or NRI antidepressant). In some embodiments, a maintenance dose includes one or more of a maintenance dose of psilocybin, CBD, a neurotransmitter activity modulator (e.g., a tricyclic, SSRI, SNRI, or NRI antidepressant), and a mushroom blend.

A maintenance dose may include an amount of synthetic psilocybin or a derivative thereof that provides from about 0.01 mg/day up to about 10 mg/day of the psilocybin or the derivative thereof. This includes about 0.01 mg/day, about 0.02 mg/day, about 0.03 mg/day, about 0.04 mg/day, about 0.05 mg/day, about 0.06 mg/day, about 0.07 mg/day, about 0.08 mg/day, about 0.09 mg/day, about 0.1 mg/day, about 0.11 mg/day, about 0.12 mg/day, about 0.13 mg/day, about 0.14 mg/day, about 0.15 mg/day, about 0.16 mg/day, about 0.17 mg/day, about 0.18 mg/day, about 0.19 mg/day, about 0.2 mg/day, about 0.21 mg/day, about 0.22 mg/day, about 0.23 mg/day, about 0.24 mg/day, about 0.25 mg/day, about 0.26 mg/day, about 0.27 mg/day, about 0.28 mg/day, about 0.29 mg/day, about 0.3 mg/day, about 0.31 mg/day, about 0.32 mg/day, about 0.33 mg/day, about 0.34 mg/day, about 0.35 mg/day, about 0.36 mg/day, about 0.37 mg/day, about 0.38 mg/day, about 0.39 mg/day, about 0.4 mg/day, about 0.41 mg/day, about 0.42 mg/day, about 0.43 mg/day, about 0.44 mg/day, about 0.45 mg/day, about 0.46 mg/day, about 0.47 mg/day, about 0.48 mg/day, about 0.49 mg/day, about 0.5 mg/day, about 0.51 mg/day, about 0.52 mg/day, about 0.53 mg/day, about 0.54 mg/day, about 0.55 mg/day, about 0.56 mg/day, about 0.57 mg/day, about 0.58 mg/day, about 0.59 mg/day, about 0.6 mg/day, about 0.7 mg/day, about 0.8 mg/day, about 0.9 mg/day, about 1 mg/day, about 2 mg/day, about 3 mg/day, about 4 mg/day, about 5 mg/day, about 6 mg/day, about 7 mg/day, about 8 mg/day, about 9 mg/day, or about 10 mg/day, and any amounts therebetween. For example, the dosage form may include an amount of psilocybin that provides from about 0.01 mg/day up to about 6 mg/day psilocybin. Other non-limiting ranges include about 0.1 mg/day up to about 9 mg/day, about 0.1 mg/day up to about 8 mg/day, about 0.1 mg/day up to about 7 mg/day, about 0.1 mg/day up to about 6 mg/day, about 0.1 mg/day up to about 5 mg/day, about 0.1 mg/day up to about 4 mg/day, about 0.1 mg/day up to about 3 mg/day, about 0.1 mg/day up to about 2 mg/day, about 0.1 mg/day up to about 1 mg/day, about 0.1 mg/day up to about 0.9 mg/day, about 0.1 mg/day up to about 0.8 mg/day, about 0.1 mg/day up to about 0.7 mg/day, about 0.1 mg/day up to about 0.6 mg/day, about 0.1 mg/day up to about 0.5 mg/day, about 0.1 mg/day up to about 0.4 mg/day, 0.2 mg/day up to about 9 mg/day, about 0.2 mg/day up to about 8 mg/day, about 0.2 mg/day up to about 7 mg/day, about 0.2 mg/day up to about 6 mg/day, about 0.2 mg/day up to about 5 mg/day, about 0.2 mg/day up to about 4 mg/day, about 0.2 mg/day up to about 3 mg/day, about 0.2 mg/day up to about 2 mg/day, about 0.2 mg/day up to about 1 mg/day, about 0.2 mg/day up to about 0.9 mg/day, about 0.2 mg/day up to about 0.8 mg/day, about 0.2 mg/day up to about 0.7 mg/day, about 0.2 mg/day up to about 0.6 mg/day, about 0.2 mg/day up to about 0.5 mg/day, about 0.2 mg/day up to about 0.4 mg/day, about 0.2 mg/day up to about 0.3 mg/day, about 0.25 mg/day up to about 0.4 mg/day, about 0.3 mg/day up to about 0.4 mg/day, about 0.5 mg/day up to about 9 mg/day, about 0.5 mg/day up to about 8 mg/day, about 0.5 mg/day up to about 7 mg/day, about 0.5 mg/day up to about 6 mg/day, about 0.5 mg/day up to about 5 mg/day, about 0.5 mg/day up to about 4 mg/day, about 0.5 mg/day up to about 3 mg/day, about 0.5 mg/day up to about 2 mg/day, about 0.5 mg/day up to about 1 mg/day, about 1 mg/day up to about 9 mg/day, about 1 mg/day up to about 8 mg/day, about 1 mg/day up to about 7 mg/day, about 1 mg/day up to about 6 mg/day, about 1 mg/day up to about 5 mg/day, 1 mg/day up to about 4 mg/day, about 1 mg/day up to about 3 mg/day, about 1 mg/day up to about 2 mg/day of psilocybin.

In some embodiments, a maintenance dose of synthetic psilocybin or derivative thereof or psilocin or derivative thereof, can be calculated based on the subject's body weight. In some embodiments, the composition comprises about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, or about 0.1 mg/kg of the subject's body weight. In some embodiments, a maintenance dose of synthetic psilocybin or a derivative thereof, or psilocin or a derivative thereof can comprise about 0.01 mg/kg to about 0.1 mg/kg, about 0.02 mg/kg to about 0.1 mg/kg, about 0.03 mg/kg to about 0.1 mg/kg, about 0.04 mg/kg to about 0.1 mg/kg, about 0.01 mg/kg to about 0.09 mg/kg, about 0.01 mg/kg to about 0.08 mg/kg, about 0.01 mg/kg to about 0.07 mg/kg, about 0.01 mg/kg to about 0.06 mg/kg, about 0.01 mg/kg to about 0.05 mg/kg, about 0.01 mg/kg to about 0.04 mg/kg, about 0.02 mg/kg to about 0.08 mg/kg, about 0.03 mg/kg to about 0.07 mg/kg, about 0.03 mg/kg to about 0.06 mg/kg, about 0.03 mg/kg to about 0.05 mg/kg, about 0.03 mg/kg to about 0.045 mg/kg, or about 0.03 mg/kg to about 0.04 mg/kg of the subject's body weight.

A maintenance dose may include natural psilocybin or a derivative thereof in the form of a dried mushroom, such as Psilocybe cubensis, or dried mushroom blend may include an amount of from about 0.05 g to about 0.6 g. This includes about 0.05 g, about 0.06 g, about 0.07 g, about 0.08 g, about 0.09 g, about 0.1 g, about 0.12 g, about 0.14 g, about 0.16 g, about 0.18 g, about 0.2 g, about 0.22 g, about 0.24 g, about 0.26 g, about 0.28 g, about 0.30 g, about 0.32 g, about 0.34 g, about 0.36 g, about 0.38 g, about 0.4 g, about 0.42 g, about 0.44 g, about 0.46 g, about 0.48 g, about 0.5 g, about 0.52 g, about 0.54 g, about 0.56 g, about 0.58 g, about 0.6 g. A maintenance dose of natural psilocybin or a derivative thereof may include psilocybin or a derivative thereof in an amount from about 0.05 g mg to about 0.6 g, about 0.1 g to about 0.6 g, about 0.1 g to about 0.5 g, about 0.1 g to about 0.4 g, about 0.1 g to about 0.3 g, about 0.1 g to about 0.2 g, about 0.2 g to about 0.6 g, about 0.2 g to about 0.5 g, about 0.2 g to about 0.4 g, about 0.2 g to about 0.3 g, about 0.3 g to about 0.6 g, about 0.3 g to about 0.5 g, about 0.3 g to about 0.4 g, about 0.4 g to about 0.6 g, about 0.4 g to about 0.5 g. In some embodiments, a maintenance dose of natural psilocybin or derivative thereof or psilocin or derivative thereof is about 0.25 g to about 0.5 g of dried mushrooms or a dried mushroom blend.

A maintenance dose of natural psilocybin or a derivative thereof, or psilocin or a derivative thereof in the form of a dried mushroom, such as Psilocybe cubensis, or dried mushroom blend can be determined based on the subject's body weight. In some embodiments, the natural psilocybin comprises about 0.001 mg/kg, about 0.002 mg/kg, about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.011 mg/kg, about 0.012 mg/kg, about 0.013 mg/kg, about 0.014 mg/kg, about 0.015 mg/kg, about 0.016 mg/kg, about 0.017 mg/kg, about 0.018 mg/kg, about 0.019 mg/kg, or about 0.02 mg/kg of the subject's body weight. In some embodiments the natural psilocybin or a derivative thereof, or psilocin or a derivative thereof is in the form of a dried mushroom or dried mushroom blend, wherein the composition comprises about 0.001 mg/kg to about 0.02 mg/kg, about 0.002 mg/kg to about 0.02 mg/kg, about 0.003 mg/kg to about 0.02 mg/kg, about 0.004 mg/kg to about 0.02 mg/kg, about 0.005 mg/kg to about 0.02 mg/kg, about 0.006 mg/kg to about 0.02 mg/kg, about 0.007 mg/kg to about 0.02 mg/kg, about 0.008 mg/kg to about 0.02 mg/kg, about 0.009 mg/kg to about 0.02 mg/kg, about 0.007 mg/kg to about 0.01 mg/kg, about 0.006 mg/kg to about 0.011 mg/kg, about 0.005 mg/kg to about 0.013 mg/kg, about 0.04 mg/kg to about 0.013 mg/kg, about 0.04 mg/kg to about 0.014 mg/kg, about 0.04 mg/kg to about 0.014 mg/kg of the subject's body weight.

A maintenance dose may include from about 1 mg to about 1000 mg of a cannabinoid. The cannabinoid in various embodiments is cannabidiol (CBD). In some embodiments, the cannabinoid is synthetic CBD. The maintenance dose may include, e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, or about 1000 mg cannabinoid (e.g., CBD), or any amount therebetween.

Thus, a maintenance dose may include a synthetic cannabinoid in an amount from about 1 mg to about 1000 mg, about 1 mg to about 600 mg, about 1 mg to about 500 mg, about 1 mg to about 400 mg, about 1 mg to about 300 mg, about 1 mg to about 200 mg, about 1 mg to about 100 mg, about 1 mg to about 50 mg, about 10 mg to about 1000 mg, about 10 mg to about 500 mg, about 10 mg to about 400 mg, about 10 mg to about 300 mg, about 10 mg to about 200 mg, about 10 mg to about 100 mg, about 100 mg to about 1000 mg, about 100 mg to about 900 mg, about 100 mg to about 800 mg, about 100 mg to about 700 mg, about 100 mg to about 600 mg, about 200 mg to about 1000 mg, about 200 mg to about 900 mg, about 200 mg to about 800 mg, about 200 mg to about 700 mg, about 200 mg to about 600 mg, about 250 mg to about 1000 mg, about 250 mg to about 900 mg, about 250 mg to about 800 mg, about 250 mg to about 700 mg, about 250 mg to about 600 mg, about 300 mg to about 1000 mg, about 300 mg to about 900 mg, about 300 mg to about 800 mg, about 300 mg to about 700 mg, about 300 mg to about 600 mg, about 350 mg to about 900 mg, about 350 mg to about 800 mg, about 350 mg to about 700 mg, about 350 mg to about 650 mg, about 350 mg to about 600 mg. In some embodiments, a maintenance dose may include a synthetic cannabinoid in an amount from about 1 mg to about 5 mg, about 1 mg to about 10 mg, about 1 mg to about 15 mg, about 1 mg to about 20 mg, about 1 mg to about 25 mg, about 1 mg to about 30 mg, about 1 mg to about 35 mg, about 1 mg to about 40 mg, about 1 mg to about 45 mg, about 1 mg to about 50 mg, about 1 mg to about 55 mg, about 1 mg to about 60 mg, about 1 mg to about 65 mg, about 1 mg to about 70 mg, about 1 mg to about 75 mg, about 1 mg to about 80 mg, about 1 mg to about 85 mg, about 1 mg to about 90 mg, about 1 mg to about 95 mg, about 1 mg to about 100 mg, about 1 mg to about 105 mg, about 1 mg to about 110 mg, about 1 mg to about 115 mg, about 1 mg to about 120 mg, about 1 mg to about 125 mg, about 1 mg to about 130 mg, about 1 mg to about 135 mg, about 1 mg to about 140 mg, about 1 mg to about 145 mg, about 1 mg to about 150 mg, about 1 mg to about 155 mg, about 1 mg to about 160 mg, about 1 mg to about 165 mg, about 1 mg to about 170 mg, about 1 mg to about 175 mg, about 1 mg to about 180 mg, about 1 mg to about 185 mg, about 1 mg to about 190 mg, about 1 mg to about 195 mg, about 1 mg to about 200 mg, about 1 mg to about 205 mg, about 1 mg to about 210 mg, about 1 mg to about 215 mg, about 1 mg to about 220 mg, about 1 mg to about 225 mg, about 1 mg to about 230 mg, about 1 mg to about 235 mg, about 1 mg to about 240 mg, about 1 mg to about 245 mg, about 1 mg to about 250 mg, about 1 mg to about 255 mg, about 1 mg to about 260 mg, about 1 mg to about 265 mg, about 1 mg to about 270 mg, about 1 mg to about 275 mg, about 1 mg to about 280 mg, about 1 mg to about 285 mg, about 1 mg to about 290 mg, about 1 mg to about 295 mg, or about 1 mg to about 300 mg.

In some embodiments, a maintenance dose of synthetic cannabidiol or derivative thereof, can be calculated based on the subject's body weight. In some embodiments, the composition comprises about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, or about 15 mg/kg of the subject's body weight. In some embodiments, the maintenance dose of synthetic cannabidiol or a derivative thereof can comprise about 1 mg/kg to about 15 mg/kg, about 1 mg/kg to about 13 mg/kg, about 1 mg/kg to about 11 mg/kg, about 2 mg/kg to about 11 mg/kg, about 3 mg/kg to about 10 mg/kg, about 4 mg/kg to about 9 mg/kg of the subject's body weight.

In some embodiments, a maintenance dose of cannabidiol or derivative thereof can be a natural cannabidiol, for example in the form of a full kief. In some embodiments, a maintenance dose of natural cannabidiol comprises about 1 to about 2000 mg of natural cannabidiol, for example, about 1 mg, about 50, about 100 mg, about 150 mg, about 200 mg, about 300 mg, about 350 mg, about 400 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1003 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1250 mg, about 1260 mg, about 1270 mg, about 1280 mg, about 1290, about 1300 mg, about 1310 mg, about 13200 mg, about 1330 mg, about 1340 mg, about 1350 mg, about 1360 mg, about 137 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg cannabinoid (e.g., CBD), or any amount therebetween.

In some embodiments, a maintenance dose may include a natural cannabinoid in an amount from about 1 mg to about 1000 mg, about 1 mg to about 600 mg, about 1 mg to about 500 mg, about 1 mg to about 400 mg, about 1 mg to about 300 mg, about 1 mg to about 200 mg, about 1 mg to about 100 mg, about 1 mg to about 50 mg, about 10 mg to about 1000 mg, about 10 mg to about 500 mg, about 10 mg to about 400 mg, about 10 mg to about 300 mg, about 10 mg to about 200 mg, about 10 mg to about 100 mg, about 100 mg to about 1000 mg, about 100 mg to about 900 mg, about 100 mg to about 800 mg, about 100 mg to about 700 mg, about 100 mg to about 600 mg, about 200 mg to about 1000 mg, about 200 mg to about 900 mg, about 200 mg to about 800 mg, about 200 mg to about 700 mg, about 200 mg to about 600 mg, about 250 mg to about 1000 mg, about 250 mg to about 900 mg, about 250 mg to about 800 mg, about 250 mg to about 700 mg, about 250 mg to about 600 mg, about 300 mg to about 1000 mg, about 300 mg to about 900 mg, about 300 mg to about 800 mg, about 300 mg to about 700 mg, about 300 mg to about 600 mg, about 350 mg to about 900 mg, about 350 mg to about 800 mg, about 350 mg to about 700 mg, about 350 mg to about 650 mg, about 350 mg to about 600 mg. In some embodiments, a maintenance dose may include a natural cannabinoid in an amount from about 1 mg to about 5 mg, about 1 mg to about 10 mg, about 1 mg to about 15 mg, about 1 mg to about 20 mg, about 1 mg to about 25 mg, about 1 mg to about 30 mg, about 1 mg to about 35 mg, about 1 mg to about 40 mg, about 1 mg to about 45 mg, about 1 mg to about 50 mg, about 1 mg to about 55 mg, about 1 mg to about 60 mg, about 1 mg to about 65 mg, about 1 mg to about 70 mg, about 1 mg to about 75 mg, about 1 mg to about 80 mg, about 1 mg to about 85 mg, about 1 mg to about 90 mg, about 1 mg to about 95 mg, about 1 mg to about 100 mg, about 1 mg to about 105 mg, about 1 mg to about 110 mg, about 1 mg to about 115 mg, about 1 mg to about 120 mg, about 1 mg to about 125 mg, about 1 mg to about 130 mg, about 1 mg to about 135 mg, about 1 mg to about 140 mg, about 1 mg to about 145 mg, about 1 mg to about 150 mg, about 1 mg to about 155 mg, about 1 mg to about 160 mg, about 1 mg to about 165 mg, about 1 mg to about 170 mg, about 1 mg to about 175 mg, about 1 mg to about 180 mg, about 1 mg to about 185 mg, about 1 mg to about 190 mg, about 1 mg to about 195 mg, about 1 mg to about 200 mg, about 1 mg to about 205 mg, about 1 mg to about 210 mg, about 1 mg to about 215 mg, about 1 mg to about 220 mg, about 1 mg to about 225 mg, about 1 mg to about 230 mg, about 1 mg to about 235 mg, about 1 mg to about 240 mg, about 1 mg to about 245 mg, about 1 mg to about 250 mg, about 1 mg to about 255 mg, about 1 mg to about 260 mg, about 1 mg to about 265 mg, about 1 mg to about 270 mg, about 1 mg to about 275 mg, about 1 mg to about 280 mg, about 1 mg to about 285 mg, about 1 mg to about 290 mg, about 1 mg to about 295 mg, or about 1 mg to about 300 mg.

In some embodiments, a maintenance dose of natural cannabidiol, can be calculated based on the subject's body weight. In some embodiments, a maintenance dose of natural cannabidiol comprises about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, or about 3.0 mg/kg of the subject's body weight. In some embodiments, a maintenance dose natural cannabidiol can comprise about 0.1 mg/kg to about 3 mg/kg, about 0.2 mg/kg to about 3 mg/kg, about 0.3 mg/kg to about 3 mg/kg, about 0.4 mg/kg to about 3 mg/kg, about 0.5 mg/kg to about 3 mg/kg, about 0.6 mg/kg to about 3 mg/kg, about 0.7 mg/kg to about 3 mg/kg, about 0.5 mg/kg to about 2.9 mg/kg, about 0.5 mg/kg to about 2.8 mg/kg, about 0.5 mg/kg to about 2.7 mg/kg, about 0.5 mg/kg to about 2.6 mg/kg, about 0.5 mg/kg to about 2.5 mg/kg, about 0.7 mg/kg to about 2.9 mg/kg, about 0.7 mg/kg to about 2.8 mg/kg, about 0.7 mg/kg to about 2.7 mg/kg, about 0.7 mg/kg to about 2.6 mg/kg, or about 0.7 mg/kg to about 2.5 mg/kg of the subject's body weight.

A maintenance dose can include the mushroom blends described above. In some embodiments, a maintenance dose can include two or more mushrooms selected from the group consisting of Inonotus obliquus (also known as the Chaga mushroom), Agaricus subrufescens (also known as Agaricus blazei or the almond mushroom), Laricifomes officinalis (also known as the Agarikon mushroom), Trametes versicolor (also known as Turkey Tail), Ganoderma lingzhi (also known as the reishi mushroom), Schizophyllum commune (also known as the suehirotake mushroom or split-fold mushroom), Hericium erinaceus (also known as the Lion's Mane mushroom), Wolfiporia extensa (also known as the poria mushroom), Cordyceps sp. (such as, but not limited to, Cordyceps militaris), Fomes fomentarius (also known as the True Tinder Polypore), Pleurotus ostreatus (also known as the oyster mushroom), Phellinus linteus (also known as the Mesima mushroom), Grifola frondosa (also known as the maitake mushroom), and Lentinula edodes (also known as shiitake mushroom), various sub-combinations thereof, and, optionally, Psilocybe cubensis.

Thus, a maintenance dose can include mushroom blends of Inonotus obliquus, Agaricus subrufescens, Laricifomes officinalis, Trametes versicolor, Ganoderma lingzhi, Schizophyllum commune, Hericium erinaceus, Wolfiporia extensa, Cordyceps sp., Fomes fomentarius, Pleurotus ostreatus, Phellinus linteus, Grifola frondosa, and Lentinula edodes, or, in embodiments comprising Psilocybe cubensis, may comprise, consist essentially of, or consist of Inonotus obliquus, Agaricus subrufescens, Laricifomes officinalis, Trametes versicolor, Ganoderma lingzhi, Schizophyllum commune, Hericium erinaceus, Wolfiporia extensa, Cordyceps sp., Fomes fomentarius, Pleurotus ostreatus, Phellinus linteus, Grifola frondosa, and Lentinula edodes, or various sub-combinations thereof. In some embodiments, the mushroom blends further comprise dried Psilocybe cubensis.

A maintenance dose can include mushroom blends of Inonotus obliquus (also known as the Chaga mushroom), Laricifomes officinalis (also known as the Agarikon mushroom), Trametes versicolor (also known as Turkey Tail), Ganoderma lingzhi (also known as the reishi mushroom), Hericium erinaceus (also known as the Lion's Mane mushroom), Cordyceps sp. (such as, but not limited to, Cordyceps militaris). The mushroom blend may consist essentially of or consist of Inonotus obliquus, Laricifomes officinalis, Trametes versicolor, Ganoderma lingzhi, Hericium erinaceus, and Cordyceps sp.

A maintenance dose can include mushroom blends of Hericium erinaceus (also known as the Lion's Mane mushroom) and Ganoderma lingzhi (also known as the reishi mushroom). The mushroom blend may consist essentially of or consist of Ganoderma lingzhi (also known as the reishi mushroom), Hericium erinaceus (also known as the Lion's Mane mushroom).

A maintenance doses which include two or more of the mushrooms described above can include any of the mushroom blend combinations and any of the mushroom blend amounts described above under “Compositions—Mushroom blends”.

A maintenance dose may include an amount of a neurotransmitter activity modulator or a derivative thereof (e.g., a tricyclic, SSRI, SNRI, or NRI antidepressant) that provides from about 1 mg to about 600 mg of the neurotransmitter activity modulator. In some embodiments the neurotransmitter activity modulator or derivative thereof comprises about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, or about 600 mg. In some embodiments, the amount of neurotransmitter activity modulator can comprise about 1 mg to about 5 mg, about 1 mg to about 10 mg, about 1 mg to about 15 mg, about 1 mg to about 20 mg, about 1 mg to about 25 mg, about 1 mg to about 30 mg, about 1 mg to about 35 mg, about 1 mg to about 40 mg, about 1 mg to about 45 mg, about 1 mg to about 50 mg, about 1 mg to about 55 mg, about 1 mg to about 60 mg, about 1 mg to about 65 mg, about 1 mg to about 70 mg, about 1 mg to about 75 mg, about 1 mg to about 80 mg, about 1 mg to about 85 mg, about 1 mg to about 90 mg, about 1 mg to about 95 mg, about 1 mg to about 100 mg, about 1 mg to about 105 mg, about 1 mg to about 110 mg, about 1 mg to about 115 mg, about 1 mg to about 120 mg, about 1 mg to about 125 mg, about 1 mg to about 130 mg, about 1 mg to about 135 mg, about 1 mg to about 140 mg, about 1 mg to about 145 mg, about 1 mg to about 150 mg, about 1 mg to about 155 mg, about 1 mg to about 160 mg, about 1 mg to about 165 mg, about 1 mg to about 170 mg, about 1 mg to about 175 mg, about 1 mg to about 180 mg, about 1 mg to about 185 mg, about 1 mg to about 190 mg, about 1 mg to about 195 mg, about 1 mg to about 200 mg, about 1 mg to about 205 mg, about 1 mg to about 210 mg, about 1 mg to about 215 mg, about 1 mg to about 220 mg, about 1 mg to about 225 mg, about 1 mg to about 230 mg, about 1 mg to about 235 mg, about 1 mg to about 240 mg, about 1 mg to about 245 mg, about 1 mg to about 250 mg, about 1 mg to about 255 mg, about 1 mg to about 260 mg, about 1 mg to about 265 mg, about 1 mg to about 270 mg, about 1 mg to about 275 mg, about 1 mg to about 280 mg, about 1 mg to about 285 mg, about 1 mg to about 290 mg, about 1 mg to about 295 mg, about 1 mg to about 300 mg, about 1 mg to about 305 mg, about 1 mg to about 310 mg, about 1 mg to about 315 mg, about 1 mg to about 320 mg, about 1 mg to about 325 mg, about 1 mg to about 330 mg, about 1 mg to about 335 mg, about 1 mg to about 340 mg, about 1 mg to about 345 mg, about 1 mg to about 350 mg, about 1 mg to about 355 mg, about 1 mg to about 360 mg, about 1 mg to about 365 mg, about 1 mg to about 370 mg, about 1 mg to about 375 mg, about 1 mg to about 380 mg, about 1 mg to about 385 mg, about 1 mg to about 390 mg, about 1 mg to about 395 mg, about 1 mg to about 400 mg, about 1 mg to about 405 mg, about 1 mg to about 410 mg, about 1 mg to about 415 mg, about 1 mg to about 420 mg, about 1 mg to about 425 mg, about 1 mg to about 430 mg, about 1 mg to about 435 mg, about 1 mg to about 440 mg, about 1 mg to about 445 mg, about 1 mg to about 450 mg, about 1 mg to about 455 mg, about 1 mg to about 460 mg, about 1 mg to about 465 mg, about 1 mg to about 470 mg, about 1 mg to about 475 mg, about 1 mg to about 480 mg, about 1 mg to about 485 mg, about 1 mg to about 490 mg, about 1 mg to about 495 mg, about 1 mg to about 500 mg, about 1 mg to about 505 mg, about 1 mg to about 510 mg, about 1 mg to about 515 mg, about 1 mg to about 520 mg, about 1 mg to about 525 mg, about 1 mg to about 530 mg, about 1 mg to about 535 mg, about 1 mg to about 540 mg, about 1 mg to about 545 mg, about 1 mg to about 550 mg, about 1 mg to about 555 mg, about 1 mg to about 560 mg, about 1 mg to about 565 mg, about 1 mg to about 570 mg, about 1 mg to about 575 mg, about 1 mg to about 580 mg, about 1 mg to about 585 mg, about 1 mg to about 590 mg, about 1 mg to about 595 mg, or about 1 mg to about 600 mg.

Specifically, a maintenance dose may include about 1 mg to about 500 mg of a tricyclic antidepressant. In some embodiments, the neurotransmitter activity modulator or derivative thereof comprises about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, or about 600 mg. In some embodiments, the amount of tricyclic antidepressant or derivative thereof can comprise about 1 mg to about 5 mg, about 1 mg to about 10 mg, about 1 mg to about 15 mg, about 1 mg to about 20 mg, about 1 mg to about 25 mg, about 1 mg to about 30 mg, about 1 mg to about 35 mg, about 1 mg to about 40 mg, about 1 mg to about 45 mg, about 1 mg to about 50 mg, about 1 mg to about 55 mg, about 1 mg to about 60 mg, about 1 mg to about 65 mg, about 1 mg to about 70 mg, about 1 mg to about 75 mg, about 1 mg to about 80 mg, about 1 mg to about 85 mg, about 1 mg to about 90 mg, about 1 mg to about 95 mg, about 1 mg to about 100 mg, about 1 mg to about 105 mg, about 1 mg to about 110 mg, about 1 mg to about 115 mg, about 1 mg to about 120 mg, about 1 mg to about 125 mg, about 1 mg to about 130 mg, about 1 mg to about 135 mg, about 1 mg to about 140 mg, about 1 mg to about 145 mg, about 1 mg to about 150 mg, about 1 mg to about 155 mg, about 1 mg to about 160 mg, about 1 mg to about 165 mg, about 1 mg to about 170 mg, about 1 mg to about 175 mg, about 1 mg to about 180 mg, about 1 mg to about 185 mg, about 1 mg to about 190 mg, about 1 mg to about 195 mg, about 1 mg to about 200 mg, about 1 mg to about 205 mg, about 1 mg to about 210 mg, about 1 mg to about 215 mg, about 1 mg to about 220 mg, about 1 mg to about 225 mg, about 1 mg to about 230 mg, about 1 mg to about 235 mg, about 1 mg to about 240 mg, about 1 mg to about 245 mg, about 1 mg to about 250 mg, about 1 mg to about 255 mg, about 1 mg to about 260 mg, about 1 mg to about 265 mg, about 1 mg to about 270 mg, about 1 mg to about 275 mg, about 1 mg to about 280 mg, about 1 mg to about 285 mg, about 1 mg to about 290 mg, about 1 mg to about 295 mg, about 1 mg to about 300 mg, about 1 mg to about 305 mg, about 1 mg to about 310 mg, about 1 mg to about 315 mg, about 1 mg to about 320 mg, about 1 mg to about 325 mg, about 1 mg to about 330 mg, about 1 mg to about 335 mg, about 1 mg to about 340 mg, about 1 mg to about 345 mg, about 1 mg to about 350 mg, about 1 mg to about 355 mg, about 1 mg to about 360 mg, about 1 mg to about 365 mg, about 1 mg to about 370 mg, about 1 mg to about 375 mg, about 1 mg to about 380 mg, about 1 mg to about 385 mg, about 1 mg to about 390 mg, about 1 mg to about 395 mg, about 1 mg to about 400 mg, about 1 mg to about 405 mg, about 1 mg to about 410 mg, about 1 mg to about 415 mg, about 1 mg to about 420 mg, about 1 mg to about 425 mg, about 1 mg to about 430 mg, about 1 mg to about 435 mg, about 1 mg to about 440 mg, about 1 mg to about 445 mg, about 1 mg to about 450 mg, about 1 mg to about 455 mg, about 1 mg to about 460 mg, about 1 mg to about 465 mg, about 1 mg to about 470 mg, about 1 mg to about 475 mg, about 1 mg to about 480 mg, about 1 mg to about 485 mg, about 1 mg to about 490 mg, about 1 mg to about 495 mg, about 1 mg to about 500 mg, about 1 mg to about 505 mg, about 1 mg to about 510 mg, about 1 mg to about 515 mg, about 1 mg to about 520 mg, about 1 mg to about 525 mg, about 1 mg to about 530 mg, about 1 mg to about 535 mg, about 1 mg to about 540 mg, about 1 mg to about 545 mg, about 1 mg to about 550 mg, about 1 mg to about 555 mg, about 1 mg to about 560 mg, about 1 mg to about 565 mg, about 1 mg to about 570 mg, about 1 mg to about 575 mg, about 1 mg to about 580 mg, about 1 mg to about 585 mg, about 1 mg to about 590 mg, about 1 mg to about 595 mg, or about 1 mg to about 600 mg.

In some embodiments, the tricyclic antidepressant can include between about 1 mg and about 300 mg imipramine. In some embodiments, the imipramine or derivative thereof comprises about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 225 mg, about 250 mg, about 275 mg, or about 300 mg. In some embodiments, the amount of imipramine or derivative thereof can comprise about 1 mg to about 5 mg, about 1 mg to about 10 mg, about 1 mg to about 15 mg, about 1 mg to about 20 mg, about 1 mg to about 25 mg, about 1 mg to about 30 mg, about 1 mg to about 35 mg, about 1 mg to about 40 mg, about 1 mg to about 45 mg, about 1 mg to about 50 mg, about 1 mg to about 55 mg, about 1 mg to about 60 mg, about 1 mg to about 65 mg, about 1 mg to about 70 mg, about 1 mg to about 75 mg, about 1 mg to about 80 mg, about 1 mg to about 85 mg, about 1 mg to about 90 mg, about 1 mg to about 95 mg, about 1 mg to about 100 mg, about 1 mg to about 105 mg, about 1 mg to about 110 mg, about 1 mg to about 115 mg, about 1 mg to about 120 mg, about 1 mg to about 125 mg, about 1 mg to about 130 mg, about 1 mg to about 135 mg, about 1 mg to about 140 mg, about 1 mg to about 145 mg, about 1 mg to about 150 mg, about 1 mg to about 155 mg, about 1 mg to about 160 mg, about 1 mg to about 165 mg, about 1 mg to about 170 mg, about 1 mg to about 175 mg, about 1 mg to about 180 mg, about 1 mg to about 185 mg, about 1 mg to about 190 mg, about 1 mg to about 195 mg, about 1 mg to about 200 mg, about 1 mg to about 205 mg, about 1 mg to about 210 mg, about 1 mg to about 215 mg, about 1 mg to about 220 mg, about 1 mg to about 225 mg, about 1 mg to about 230 mg, about 1 mg to about 235 mg, about 1 mg to about 240 mg, about 1 mg to about 245 mg, about 1 mg to about 250 mg, about 1 mg to about 255 mg, about 1 mg to about 260 mg, about 1 mg to about 265 mg, about 1 mg to about 270 mg, about 1 mg to about 275 mg, about 1 mg to about 280 mg, about 1 mg to about 285 mg, about 1 mg to about 290 mg, about 1 mg to about 295 mg, or about 1 mg to about 300 mg.

A maintenance dose may include about 1 mg to about 600 mg of an SSRI. In some embodiments, the SSRI or derivative thereof comprises about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, or about 600 mg. In some embodiments, the amount of SSRI or derivative thereof can comprise about 1 mg to about 5 mg, about 1 mg to about 10 mg, about 1 mg to about 15 mg, about 1 mg to about 20 mg, about 1 mg to about 25 mg, about 1 mg to about 30 mg, about 1 mg to about 35 mg, about 1 mg to about 40 mg, about 1 mg to about 45 mg, about 1 mg to about 50 mg, about 1 mg to about 55 mg, about 1 mg to about 60 mg, about 1 mg to about 65 mg, about 1 mg to about 70 mg, about 1 mg to about 75 mg, about 1 mg to about 80 mg, about 1 mg to about 85 mg, about 1 mg to about 90 mg, about 1 mg to about 95 mg, about 1 mg to about 100 mg, about 1 mg to about 105 mg, about 1 mg to about 110 mg, about 1 mg to about 115 mg, about 1 mg to about 120 mg, about 1 mg to about 125 mg, about 1 mg to about 130 mg, about 1 mg to about 135 mg, about 1 mg to about 140 mg, about 1 mg to about 145 mg, about 1 mg to about 150 mg, about 1 mg to about 155 mg, about 1 mg to about 160 mg, about 1 mg to about 165 mg, about 1 mg to about 170 mg, about 1 mg to about 175 mg, about 1 mg to about 180 mg, about 1 mg to about 185 mg, about 1 mg to about 190 mg, about 1 mg to about 195 mg, about 1 mg to about 200 mg, about 1 mg to about 205 mg, about 1 mg to about 210 mg, about 1 mg to about 215 mg, about 1 mg to about 220 mg, about 1 mg to about 225 mg, about 1 mg to about 230 mg, about 1 mg to about 235 mg, about 1 mg to about 240 mg, about 1 mg to about 245 mg, about 1 mg to about 250 mg, about 1 mg to about 255 mg, about 1 mg to about 260 mg, about 1 mg to about 265 mg, about 1 mg to about 270 mg, about 1 mg to about 275 mg, about 1 mg to about 280 mg, about 1 mg to about 285 mg, about 1 mg to about 290 mg, about 1 mg to about 295 mg, about 1 mg to about 300 mg, about 1 mg to about 305 mg, about 1 mg to about 310 mg, about 1 mg to about 315 mg, about 1 mg to about 320 mg, about 1 mg to about 325 mg, about 1 mg to about 330 mg, about 1 mg to about 335 mg, about 1 mg to about 340 mg, about 1 mg to about 345 mg, about 1 mg to about 350 mg, about 1 mg to about 355 mg, about 1 mg to about 360 mg, about 1 mg to about 365 mg, about 1 mg to about 370 mg, about 1 mg to about 375 mg, about 1 mg to about 380 mg, about 1 mg to about 385 mg, about 1 mg to about 390 mg, about 1 mg to about 395 mg, about 1 mg to about 400 mg, about 1 mg to about 405 mg, about 1 mg to about 410 mg, about 1 mg to about 415 mg, about 1 mg to about 420 mg, about 1 mg to about 425 mg, about 1 mg to about 430 mg, about 1 mg to about 435 mg, about 1 mg to about 440 mg, about 1 mg to about 445 mg, about 1 mg to about 450 mg, about 1 mg to about 455 mg, about 1 mg to about 460 mg, about 1 mg to about 465 mg, about 1 mg to about 470 mg, about 1 mg to about 475 mg, about 1 mg to about 480 mg, about 1 mg to about 485 mg, about 1 mg to about 490 mg, about 1 mg to about 495 mg, about 1 mg to about 500 mg, about 1 mg to about 505 mg, about 1 mg to about 510 mg, about 1 mg to about 515 mg, about 1 mg to about 520 mg, about 1 mg to about 525 mg, about 1 mg to about 530 mg, about 1 mg to about 535 mg, about 1 mg to about 540 mg, about 1 mg to about 545 mg, about 1 mg to about 550 mg, about 1 mg to about 555 mg, about 1 mg to about 560 mg, about 1 mg to about 565 mg, about 1 mg to about 570 mg, about 1 mg to about 575 mg, about 1 mg to about 580 mg, about 1 mg to about 585 mg, about 1 mg to about 590 mg, about 1 mg to about 595 mg, or about 1 mg to about 600 mg.

A maintenance dose may include about 1 mg to about 600 mg of an SNRI. In some embodiments, the SNRI or derivative thereof comprises about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, or about 600 mg. In some embodiments, the amount of SNRI or derivative thereof can comprise about 1 mg to about 5 mg, about 1 mg to about 10 mg, about 1 mg to about 15 mg, about 1 mg to about 20 mg, about 1 mg to about 25 mg, about 1 mg to about 30 mg, about 1 mg to about 35 mg, about 1 mg to about 40 mg, about 1 mg to about 45 mg, about 1 mg to about 50 mg, about 1 mg to about 55 mg, about 1 mg to about 60 mg, about 1 mg to about 65 mg, about 1 mg to about 70 mg, about 1 mg to about 75 mg, about 1 mg to about 80 mg, about 1 mg to about 85 mg, about 1 mg to about 90 mg, about 1 mg to about 95 mg, about 1 mg to about 100 mg, about 1 mg to about 105 mg, about 1 mg to about 110 mg, about 1 mg to about 115 mg, about 1 mg to about 120 mg, about 1 mg to about 125 mg, about 1 mg to about 130 mg, about 1 mg to about 135 mg, about 1 mg to about 140 mg, about 1 mg to about 145 mg, about 1 mg to about 150 mg, about 1 mg to about 155 mg, about 1 mg to about 160 mg, about 1 mg to about 165 mg, about 1 mg to about 170 mg, about 1 mg to about 175 mg, about 1 mg to about 180 mg, about 1 mg to about 185 mg, about 1 mg to about 190 mg, about 1 mg to about 195 mg, about 1 mg to about 200 mg, about 1 mg to about 205 mg, about 1 mg to about 210 mg, about 1 mg to about 215 mg, about 1 mg to about 220 mg, about 1 mg to about 225 mg, about 1 mg to about 230 mg, about 1 mg to about 235 mg, about 1 mg to about 240 mg, about 1 mg to about 245 mg, about 1 mg to about 250 mg, about 1 mg to about 255 mg, about 1 mg to about 260 mg, about 1 mg to about 265 mg, about 1 mg to about 270 mg, about 1 mg to about 275 mg, about 1 mg to about 280 mg, about 1 mg to about 285 mg, about 1 mg to about 290 mg, about 1 mg to about 295 mg, about 1 mg to about 300 mg, about 1 mg to about 305 mg, about 1 mg to about 310 mg, about 1 mg to about 315 mg, about 1 mg to about 320 mg, about 1 mg to about 325 mg, about 1 mg to about 330 mg, about 1 mg to about 335 mg, about 1 mg to about 340 mg, about 1 mg to about 345 mg, about 1 mg to about 350 mg, about 1 mg to about 355 mg, about 1 mg to about 360 mg, about 1 mg to about 365 mg, about 1 mg to about 370 mg, about 1 mg to about 375 mg, about 1 mg to about 380 mg, about 1 mg to about 385 mg, about 1 mg to about 390 mg, about 1 mg to about 395 mg, about 1 mg to about 400 mg, about 1 mg to about 405 mg, about 1 mg to about 410 mg, about 1 mg to about 415 mg, about 1 mg to about 420 mg, about 1 mg to about 425 mg, about 1 mg to about 430 mg, about 1 mg to about 435 mg, about 1 mg to about 440 mg, about 1 mg to about 445 mg, about 1 mg to about 450 mg, about 1 mg to about 455 mg, about 1 mg to about 460 mg, about 1 mg to about 465 mg, about 1 mg to about 470 mg, about 1 mg to about 475 mg, about 1 mg to about 480 mg, about 1 mg to about 485 mg, about 1 mg to about 490 mg, about 1 mg to about 495 mg, about 1 mg to about 500 mg, about 1 mg to about 505 mg, about 1 mg to about 510 mg, about 1 mg to about 515 mg, about 1 mg to about 520 mg, about 1 mg to about 525 mg, about 1 mg to about 530 mg, about 1 mg to about 535 mg, about 1 mg to about 540 mg, about 1 mg to about 545 mg, about 1 mg to about 550 mg, about 1 mg to about 555 mg, about 1 mg to about 560 mg, about 1 mg to about 565 mg, about 1 mg to about 570 mg, about 1 mg to about 575 mg, about 1 mg to about 580 mg, about 1 mg to about 585 mg, about 1 mg to about 590 mg, about 1 mg to about 595 mg, or about 1 mg to about 600 mg.

A maintenance dose may include about 1 mg to about 600 mg of an NRI. In some embodiments, the NRI or derivative thereof comprises about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, or about 600 mg. In some embodiments, the amount of NRI or derivative thereof can comprise about 1 mg to about 5 mg, about 1 mg to about 10 mg, about 1 mg to about 15 mg, about 1 mg to about 20 mg, about 1 mg to about 25 mg, about 1 mg to about 30 mg, about 1 mg to about 35 mg, about 1 mg to about 40 mg, about 1 mg to about 45 mg, about 1 mg to about 50 mg, about 1 mg to about 55 mg, about 1 mg to about 60 mg, about 1 mg to about 65 mg, about 1 mg to about 70 mg, about 1 mg to about 75 mg, about 1 mg to about 80 mg, about 1 mg to about 85 mg, about 1 mg to about 90 mg, about 1 mg to about 95 mg, about 1 mg to about 100 mg, about 1 mg to about 105 mg, about 1 mg to about 110 mg, about 1 mg to about 115 mg, about 1 mg to about 120 mg, about 1 mg to about 125 mg, about 1 mg to about 130 mg, about 1 mg to about 135 mg, about 1 mg to about 140 mg, about 1 mg to about 145 mg, about 1 mg to about 150 mg, about 1 mg to about 155 mg, about 1 mg to about 160 mg, about 1 mg to about 165 mg, about 1 mg to about 170 mg, about 1 mg to about 175 mg, about 1 mg to about 180 mg, about 1 mg to about 185 mg, about 1 mg to about 190 mg, about 1 mg to about 195 mg, about 1 mg to about 200 mg, about 1 mg to about 205 mg, about 1 mg to about 210 mg, about 1 mg to about 215 mg, about 1 mg to about 220 mg, about 1 mg to about 225 mg, about 1 mg to about 230 mg, about 1 mg to about 235 mg, about 1 mg to about 240 mg, about 1 mg to about 245 mg, about 1 mg to about 250 mg, about 1 mg to about 255 mg, about 1 mg to about 260 mg, about 1 mg to about 265 mg, about 1 mg to about 270 mg, about 1 mg to about 275 mg, about 1 mg to about 280 mg, about 1 mg to about 285 mg, about 1 mg to about 290 mg, about 1 mg to about 295 mg, about 1 mg to about 300 mg, about 1 mg to about 305 mg, about 1 mg to about 310 mg, about 1 mg to about 315 mg, about 1 mg to about 320 mg, about 1 mg to about 325 mg, about 1 mg to about 330 mg, about 1 mg to about 335 mg, about 1 mg to about 340 mg, about 1 mg to about 345 mg, about 1 mg to about 350 mg, about 1 mg to about 355 mg, about 1 mg to about 360 mg, about 1 mg to about 365 mg, about 1 mg to about 370 mg, about 1 mg to about 375 mg, about 1 mg to about 380 mg, about 1 mg to about 385 mg, about 1 mg to about 390 mg, about 1 mg to about 395 mg, about 1 mg to about 400 mg, about 1 mg to about 405 mg, about 1 mg to about 410 mg, about 1 mg to about 415 mg, about 1 mg to about 420 mg, about 1 mg to about 425 mg, about 1 mg to about 430 mg, about 1 mg to about 435 mg, about 1 mg to about 440 mg, about 1 mg to about 445 mg, about 1 mg to about 450 mg, about 1 mg to about 455 mg, about 1 mg to about 460 mg, about 1 mg to about 465 mg, about 1 mg to about 470 mg, about 1 mg to about 475 mg, about 1 mg to about 480 mg, about 1 mg to about 485 mg, about 1 mg to about 490 mg, about 1 mg to about 495 mg, about 1 mg to about 500 mg, about 1 mg to about 505 mg, about 1 mg to about 510 mg, about 1 mg to about 515 mg, about 1 mg to about 520 mg, about 1 mg to about 525 mg, about 1 mg to about 530 mg, about 1 mg to about 535 mg, about 1 mg to about 540 mg, about 1 mg to about 545 mg, about 1 mg to about 550 mg, about 1 mg to about 555 mg, about 1 mg to about 560 mg, about 1 mg to about 565 mg, about 1 mg to about 570 mg, about 1 mg to about 575 mg, about 1 mg to about 580 mg, about 1 mg to about 585 mg, about 1 mg to about 590 mg, about 1 mg to about 595 mg, or about 1 mg to about 600 mg. In some embodiments, the NRI or derivative thereof can comprise about 5 mg to about 600 mg, about 10 mg to about 600 mg, about 15 mg to about 600 mg, about 20 mg to about 600 mg, about 25 mg to about 600 mg, about 30 mg to about 600 mg, about 35 mg to about 600 mg, about 40 mg to about 600 mg, about 45 mg to about 600 mg, about 50 mg to about 600 mg, about 55 mg to about 600 mg, about 60 mg to about 600 mg, about 65 mg to about 600 mg, about 70 mg to about 600 mg, about 75 mg to about 600 mg, about 80 mg to about 600 mg, about 85 mg to about 600 mg, about 90 mg to about 600 mg, about 95 mg to about 600 mg, about 100 mg to about 600 mg, about 105 mg to about 600 mg, about 110 mg to about 600 mg, about 115 mg to about 600 mg, about 120 mg to about 600 mg, about 125 mg to about 600 mg, about 130 mg to about 600 mg, about 135 mg to about 600 mg, about 140 mg to about 600 mg, about 145 mg to about 600 mg, or about 150 mg to about 600 mg.

### Schedules

In various embodiments, the loading dose is administered once about every 1 week, 2 weeks, 3 weeks 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, or 35 weeks. In various embodiments, the loading dose is administered once about every 1 month, 2 months, 3 months, 4 months, 5 months, 6 months, 7 months, 8 months, 9 months, 10 months, 11 months, or 12 months. In various embodiments, the loading dose is administered only once during an indicated time period. In some embodiments, the loading dose is administered more than once during an indicated time period.

In some embodiments of the methods of the disclosure, a subject is administered a loading dose of psilocybin or a derivative thereof and is not administered any further psilocybin until a subsequent loading dose after one of the time periods specified above. In some embodiments of the disclosure, a subject is administered a loading dose of psilocybin or a derivative thereof and is not administered any further psilocybin or a derivative thereof until 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days have passed. In some embodiments a subject is administered a loading dose of psilocybin or a derivative thereof and is not administered any further psilocybin or derivative thereof until 1 month, 2 months, 3 months, 4 months, 5 months, 6 months, 7 months, 8 months, 9 months, 10 months, 11 months, or 12 months have passed.

Administration of the loading dose of psilocybin or a derivative thereof may occur one day or more before commencing the maintenance dose, such as 1, 2, 3, 4, 5, 6, 7, 8, 9, 10, 11, 12, 13, 14, 15, 16, 17, 18, 19, 20, 21, 22, 23, 24, 25, 26, 27, or 28, or more days, before commencing the maintenance dose. For example, administration of the loading dose of psilocybin or a derivative thereof may occur 1 to 28 days before commencing the maintenance dose, 1 to 14 days before commencing the maintenance dose, 1 to 7 days before commencing the maintenance dose, 1 to 3 days before commencing the maintenance dose, or 1 day before commencing the maintenance dose. In some embodiments, the loading dose of psilocybin is administered about 7 days to about 21 days before commencing an initial maintenance dose. In some embodiments, the loading dose of psilocybin is administered about 10 days to about 15 days before commencing the initial maintenance dose. In some embodiments, the loading dose of psilocybin is administered about 12 days to about 15 days before commencing an initial maintenance dose. In some embodiments, the loading dose of psilocybin is administered about 14 days before commencing an initial maintenance dose.

In specific examples, administration of the loading dose of psilocybin or a derivative thereof occurs about two weeks before commencing the maintenance dose. Additionally, or alternatively, the methods described herein may comprise administering a loading dose of psilocybin or a derivative thereof (as described above) after commencing a maintenance dose. Administration of the loading dose of psilocybin or a derivative thereof may occur one day or more after commencing the maintenance dose, such as 1, 2, 3, 4, 5, 6, 7, 8, 9, 10, 11, 12, 13, 14, 15, 16, 17, 18, 19, 20, 21, 22, 23, 24, 25, 26, 27, or 28, or more days, after commencing the weekly protocol. For example, administration of the loading dose of psilocybin or a derivative thereof may occur 1 to 28 days after commencing the maintenance dose, 1 to 14 days after commencing the maintenance dose, 1 to 7 days after commencing the maintenance dose, 1 to 3 days after commencing the maintenance dose, or 1 day after commencing the maintenance dose. In specific examples, administration of the loading dose of psilocybin or a derivative thereof occurs about two weeks after commencing the maintenance dose.

The maintenance dose of psilocybin can be administered on administered on a daily basis or on a scheduled basis. Non-limiting examples of a scheduled basis include every other day, every two days, every three days, every four days, every five days, every six days, or once a week. Other non-limiting examples of a scheduled basis include one day on: six days off, two days on: five days off, three days on: four days off, four days on: three days off, five days on: two days off, six days on: one day off. Yet further non-limiting examples of a scheduled basis include two days on: one day off, two days on: two days off, two days on: three days off, two days on—four days off, two days on: five days off, three days on: one day off, three days on: two days off, three days on: three days off, three days on: four days off, four days on: one day off, four days on—two days off, four days on: three days off, five days on: one day off, five days on: two days off, six days on: one day off. Yet further non-limiting examples of a scheduled basis include one day out of every seven days, two days out of every seven days, three days out of every seven days, four days out of every seven days, five days out of every seven days, or six out of every seven days. The method may comprise administering the composition by a weekly protocol consisting of daily administration of a maintenance dose composition for 3-5 consecutive days followed by no administration for 1-3 consecutive days. A weekly protocol or schedule is sometimes referred herein as a “maintenance therapy.” In some embodiments, the maintenance dose is administered according to a weekly regimen.

The maintenance dose of cannabinoid can be administered on administered on a daily basis or on a scheduled basis. Non-limiting examples of a scheduled basis include every other day, every two days, every three days, every four days, every five days, every six days, or once a week. Other non-limiting examples of a scheduled basis include one day on—six days off, two days on—five days off, three days on—four days off, four days on—three days off, five days on two days off, six days on—one day off. Yet further non-limiting examples of a scheduled basis include two days on—one day off, two days on—two days off, two days on—three days off, two days on—four days off, two days on—five days off, three days on—one day off, three days on—two days off, three days on—three days off, three days on—four days off, four days on—one day off, four days on—two days off, four days on—three days off, five days on—one day off, five days on—two days off, six days on—one day off. Yet further non-limiting examples of a scheduled basis include one day out of every seven days, two days out of every seven days, three days out of every seven days, four days out of every seven days, five days out of every seven days, or six days out of every seven days. Additional schedules are within the scope of the disclosure.

A maintenance dose of a neurotransmitter activity modulator (e.g., a tricyclic, SNRI, SSRI, or NRI antidepressant) can be administered on administered on a daily basis or on a scheduled basis. Non-limiting examples of a scheduled basis include every other day, every two days, every three days, every four days, every five days, every six days, or once a week. Other non-limiting examples of a scheduled basis include one day on—six days off, two days on—five days off, three days on—four days off, four days on—three days off, five days on—two days off, six days on—one day off. Yet further non-limiting examples of a scheduled basis include two days on—one day off, two days on—two days off, two days on—three days off, two days on—four days off, two days on—five days off, three days on—one day off, three days on—two days off, three days on—three days off, three days on—four days off, four days on—one day off, four days on—two days off, four days on—three days off, five days on—one day off, five days on—two days off, six days on—one day off. Yet further non-limiting examples of a scheduled basis include one day out of every seven days, two days out of every seven days, three days out of every seven days, four days out of every seven days, five days out of every seven days, or six days out of every seven days. Additional schedules are within the scope of the disclosure.

Administration of psilocybin or a derivative thereof and/or a cannabidiol or a derivative thereof may be continued on an as-needed basis, including for about or at least about 1 month, 2 months, 3 months, 4 months, 5 months, 6 months, 7 months, 8 months, 9 months, 10 months, 11 months, 12 months. For example, administering the composition as a maintenance dose as outlined above may be continued for consecutive weeks, including for 2, 3, 4, or more consecutive weeks, or for consecutive months, including for 2, 3, 4, 5, 6, 7, 8, 9, 10, 11, 12, or more consecutive months, including for 1-12 months, for 1-24 months, or longer.

### Psychological Support

The methods of the present disclosure may comprise having a subject participate in one or more pre- and/or post-administration psychological support session(s). In various embodiments, the methods of the present disclosure involve administering psychotherapy to a patient.

### Pre-Administration Psychological Support Sessions

In some embodiments, the subject participates in at least one psychological support session before administration of a composition of the present disclosure (“pre-administration psychological support session”). Administration of the composition may be referred to as an “administration session”. In various embodiments the composition administered during the administration session comprises psilocybin or a derivative thereof. In some embodiments, a pre-administration psychological support session may be held about 1 month prior to the administration session. In some embodiments, a pre-administration psychological support session may be held about 2 weeks prior to the administration. In some embodiments, a pre-administration psychological support session may be held about 1 week prior to the administration. In some embodiments, a pre-administration psychological support session may be held about 3 days prior to the administration. In some embodiments, a pre-administration psychological support session may be held about 1 day prior to the administration. In some embodiments, a pre-administration psychological support session may be held on the same day as and prior to administration.

In some embodiments, the subject may participate in one, two, three, four, five, six, seven, or eight pre-administration psychological support sessions. In some embodiments, the subject may participate in at least two pre-administration psychological support sessions. In some embodiments, the subject may participate in at least three pre-administration psychological support sessions. In some embodiments, the subject may participate in pre-administration psychological support sessions at least once per week, for at least two or three weeks prior to the administration. In some embodiments, the subject may additionally participate in a pre-administration psychological support session the day before the administration. The pre-administration psychological support sessions may be individual sessions, wherein a subject meets one-on-one with the person administering the psychological support. In some embodiments, the psychological support sessions may be group sessions, wherein more than one subject meets with a single person administering the psychological support, or more than one person administering the psychological support. In some embodiments, one or more of the subject's family members or friends may be present at the pre-administration psychological support session(s).

In some embodiments, the goals of the pre-administration session may include (i) establishing therapeutic alliance between subject and person administering the psychological support; (ii) answering the subject's questions and addressing any concerns; and/or (iii) demonstrating and practicing the skills of self-directed inquiry and experiential processing. In some embodiments, the pre-administration psychological support sessions focus on discussion of possible effects, and/or preparing subjects for a dosing session by practicing relevant therapeutic techniques to reduce avoidance and anxiety, eliciting relevant therapeutic goals, building rapport, and/or establishing therapeutic alliance. During the psychological support session, skills of self-directed inquiry and experiential processing may be demonstrated and/or practiced.

### Psychological Support During Administration

During the treatment session, the subject may be supervised by one or more trained persons administering psychological support. The person administering psychological support during the administration may be the same person administering psychological support from the subject's pre-administration psychological support session(s), or may be a different person administering psychological support. The person administering psychological support may provide psychological support to the subject as necessary. As used herein, the term “psychological support” refers to any measure(s) taken by the therapist during the administration to ensure the safety of the subject and maximize the clinical effectiveness of the administration. For example, the psychological support may be anything done by the therapist to (1) to ensure psychological safety of the subject; (2) to allow the subject's subjective experience to unfold naturally within the boundaries of the therapeutic intention set at the preparation; (3) to maintain participant's attention and awareness on the experience of the present moment thus allowing exposure and processing of the challenging emotional states and personal memories; and/or (4) to generate insights and solutions for the resolution of challenging personal situations, conflicts and traumatic experiences.

In some embodiments, the main therapeutic goals of the person administering psychological support during the administration are to (i) minimize extreme anxiety, and (ii) provide appropriate support that enables the skills and processes of self-directed inquiry and experiential processing.

In some embodiments, the level of psychological support will vary during the various stages of the subject's experience during an administration session (e.g., the initial stage, the early stage, the peak stage, and the late stage). In some embodiments, the type of psychological support will vary during the various stages of the subject's experience (e.g., the initial stage, the early stage, the peak stage, and the late stage). Because non-dual, ego-dissolution or “unitive” experiences have been shown to positively correlate with the magnitude and durability of the clinical response, the person administering psychological support will, in some embodiments, attend to such states with particular care.

In some embodiments, a subject may experience of a compromised sense of self during the subject's experience. In some embodiments, an ego dissolution experience is a spontaneously occurring state of consciousness where there is a reduction in the self-referential awareness that defines normal waking consciousness, resulting in a compromised sense of “self”. In some embodiments, a unitive experience is an experience characterized by a sense of unity or “oneness” that exceeds sensory or cognitive apprehension.

At the initial and early stage of the administration session, psychological support may be used to reduce severe and/or prolonged anxiety. Anxiety prior to or during the onset of psilocybin effects is not uncommon, and the persons administering psychological support may be specially trained to recognize and actively manage subjects through such periods of anxiety until the subject is comfortable enough to continue on their own. At the initial and early stage of the administration session, the person administering psychological support may encourage the subject to lie down, practice relaxation and breathing exercises, and/or listen to calming music. In some embodiments, the person administering psychological support may remind the subject that their primary task during this session is to simply collect new and interesting experiences which can then be discussed with the person administering psychological support after the session. The person administering psychological support may remind the participant of the purpose of the therapy and the role of experiential processing, namely allowing the participant to be open and curious to whatever arises and encountering thoughts and feelings previously unknown to them. In some embodiments, the person administering psychological support emphasizes that this process inherently requires letting go and a willing passivity to the psychedelic experience.

In some embodiments, the therapist may encourage the subject to put on an eye mask, such as a Mindfold eyeshade. In some embodiments, the therapist encourages the subject to put on the eye mask before, during, or after the onset of the psilocybin's active metabolite, psilocin, effects.

In some embodiments, the person administering psychological support may encourage the subject to put on headphones and listen to music. In some embodiments, the headphones reduce outside noise (e.g., “noise cancelling” headphones). In case of prolonged anxiety or distress, therapists may, in some embodiments, actively guide participants through such experiences without interpreting or judging the experiences or giving advice. Once participants are comfortable, the therapist may encourage them to again engage in introspection.

In some embodiments, the psychological support comprises mindfulness-based therapy or CBT cognitive behavioral therapy (CBT). In some embodiments, the psychological support is informed by a functional theory of human behavior called Perceptual Control Theory. In some embodiments, a therapist provides psychological support for approximately 4-8 hours immediately after administration of the loading composition.

### Post-Administration Psychological Support Session

In some embodiments, subjects may be encouraged to engage in post-administration integration sessions with their person administering psychological support. Integration is a process that involves processing, or embodying, a psychedelic experience within a therapeutic context. The process initially begins by the subject verbalizing and reflecting upon any experience from the administration session and discussing it openly with their person administering psychological support. Successful integration of an experience accommodates for emotional changes and comprises of translating experiences into new insights, perspectives, and subsequently new behaviors that can be used to benefit the subject's quality of life. New perspectives might in turn influence the participant's current knowledge or values and lead to new ways of relating to cognitions, emotions, behaviors, and physical experiences. In some embodiments, the goals and supportive methods used by the person administering psychological support throughout integration sessions should remain consistent, regardless of the intensity or content of the subjective experience explored by the subject.

In some embodiments, the subject participates in at least one psychological support session after administration (“post-administration psychological support session”). In some embodiments, a post-administration psychological support session may be held on the same day as the administration session, after the effects of the psilocin have substantially worn off. In some embodiments, a post-administration psychological support session may be held the day after, two days after, three days after, four days after, five days after, six days after, one week after, two weeks after, three weeks after one month after, two months after, three months after, six months after, or twelve months after the administration session. In some embodiments, the subject may participate in one, two, three, four, five, six, seven, or eight post-administration psychological support sessions. In some embodiments, the subject may participate in at least two, or at least three post-administration psychological support sessions. The post-administration psychological support sessions may be individual sessions, wherein a subject meets one-on-one with a person administering psychological support. In some embodiments, the psychological support sessions may be group sessions, wherein more than one subject meets with a single person administering psychological support, or more than one person administering psychological support. In some embodiments, one or more of the subject's family members or friends may be present at the post-administration psychological support session(s).

### Psychological Support Provided Remotely

In some embodiments, psychological support may be provided remotely to a subject. For example, a person administering psychological support providing psychological support may not be in the same room, the same building, or in the same facility as a subject. Remote psychological support may be provided, for example by telephone (i.e., by voice call), by video call or video conference, by text, or by email.

In some embodiments, a pre-administration therapy session is conducted remotely. In some embodiments, a post-administration therapy session (e.g., an integration session) is conducted remotely.

### Subject Evaluation

Various methods are available to one of skill in the art to select a subject for treatment. Such methods include assessing the physical, psychological, and/or neurological state of a subject. The methods can be used to select a subject for receiving a composition of the present disclosure and/or to monitor the subject's psychological and/or neurological condition after or while being treated for a neurological or psychological condition by one of the methods of the disclosure.

Efficacy of treatment, can be confirmed by clinical evaluation using means known in the art, such as, but not limited to, comparing results of evaluations before and after treatment, assessing functional improvement (e.g., reduction in the frequency or severity of one or more of dizziness, nervousness, headache, nausea, vomiting, depression, suicide ideation, fatigue, noise or light sensitivity, insomnia, irritability, anxiety and/or light/noise sensitivity, and/or improvement in one or more of appetite, concentration, mood, memory, sleep, overall well-being, problem-solving, and multitasking ability), as may be measured using validated subjective quality of life assessments, for example.

In some embodiments, the psychological and/or neurological state of a patient is measured using a brain scan and/or quantitative electroencephalography (qEEG). Quantitative Electroencephalography or qEEG (a method within brain mapping techniques) includes applying mathematical methods to the EEG data, mainly power spectral analyses, to obtain quantitative metrics that are associated with behavioral-cognitive brain functions. QEEG is frequently used in biomedical research for measuring the pre and post effects of mental-health interventions. In some embodiments, the extent of TBI and/or the extent of recovery from TBI can be measured using a quantitative electroencephalogram (qEEG). In some embodiments, depression associated with a TBI subject can be measured using qEEG. Quantitative EEG is a common tool for understanding the electrophysiologic abnormalities that can be present after TBI or post-concussive syndrome (PCS), which can include changes in the frequency, amplitude, coherence, power, phase, and symmetry of brain waves. In some embodiments, some of the endpoints or analyses performed include spectral analysis (spectral power), functional connectivity, including subgroups of time domain measure, frequency-domain measures, and measures calculated from the geometry of embedded data, event-related potentials, sample entropy, multiscale entropy, complexity, directionality, network geometries. A qEEG normative database contains a set of metrics computed from EEG and collected from a large number of individuals, large enough to be representative of the population. This allows a comparison of an individual's metric to the qEEG database (also referred to as qEEG testing), thus indicating whether there exists a non-typical electrophysiological marker with regard to the population. The interest lies in the potential clinical relevance in certain disorders when interpreted by an experienced professional. In some embodiments, the scores for one or more of the assessment endpoints improve after treatment with the methods or compositions described herein.

In some embodiments, the subject is evaluated using The Rivermead Post-Concussion Symptoms Questionnaire or a similar questionnaire. In some embodiments, the subject is evaluated using the Spatial Working Memory (SWM) test. In some embodiments, the subject is evaluated using the spatial working memory between errors (SWMBE) score. In some embodiments, the subject is evaluated using the spatial working memory strategy (SWMS) score.

In some embodiments, the subject is evaluated using the Rapid Visual Information Processing (RVP) test. In some embodiments, the subject is evaluated using the Paired Associates Learning (PAL) test. In some embodiments, the subject is evaluated using the Emotion Recognition Task (ERT) test. In some embodiments, the subject is evaluated using the Intra-Extra Dimensional Set Shift (IED) test. In some embodiments, the subject is evaluated using the One Touch Stockings (OTS) of Cambridge test. In some embodiments, the subject's verbal fluency is evaluated. In some embodiments, the subject is evaluated using the Digit Span Forward (DSF) test. In some embodiments, the subject is evaluated using the Five Dimension Altered States of Consciousness questionnaire (5D-ASC). In some embodiments, the subject is evaluated using the Positive and Negative Affect Schedule (PANAS). In some embodiments, the subject is evaluated using the NEO-Five Factor Inventory (NEO-FFI) test. In some embodiments, the subject is evaluated using the Symptom Checklist-90 item (SCL-90) questionnaire. In some embodiments, the subject is evaluated using the Life Changes Inventory (LCI) questionnaire. In some embodiments, the subject is evaluated using the Social Cognition Panel scales.

In some embodiments, the subject is evaluated using the Pictorial Empathy Test (PET).

In some embodiments, the subject is evaluated using the Reading the Mind in the Eyes Test (RMET). In some embodiments, after treating according to the methods of the disclosure, one or more of the subject's Social Cognition Panel Scales Score, i.e., PET, RMET, SVO, TEQ, and/or SSR score), improves by about 5%, about 10%, about 15%, about 20%, about 25%, about 30%, about 35%, about 40%, about 45%, about 50%, about 55%, about 60%, about 65%, about 70%, about 75% about 80%, about 85%, about 90%, about 95%, or about 100%, about 110%, about 120%, about 130%, about 140%, about 150%, about 160%, about 170%, about 180% about 190% about 200% about 210% about 220% about 230% about 2400%, about 250%, about 260%, about 270%, about 280%, about 290%, or about 300%, or more, compared to prior to treatment.

In some embodiments, the subject is evaluated using the Toronto Empathy Questionnaire (TEQ). In some embodiments, the subject is evaluated using the Scale of Social Responsibility (SSR). In some embodiments, the subject is evaluated using the Sheehan Suicidality Tracking Scale (SSTS). In some embodiments, the subject is evaluated using the Tellegen Absorption Scale.

In some embodiments, the subject is physically examined. A physical examination, includes, but is not limited to, an examination of the subject's general appearance, including an examination of the skin, neck, eyes, ears, nose, throat, heart, lungs, abdomen, lymph nodes, extremities and musculoskeletal system. In some embodiments, body weight and height of a subject are assessed. In some embodiments, body mass index is considered.

In some embodiments, the subject is evaluated using an electrocardiogram (ECG).

In some embodiments, vital signs of a subject are used to evaluate safety and/or efficacy of a composition. Vital signs include, but are not limited to, blood pressure (BP), respiratory rate, oral body temperature, and pulse. In some embodiments, blood pressure is taken after a subject has been sitting down for at least three minutes.

In some embodiments, clinical laboratory tests are utilized to evaluate the safety and/or efficacy of a composition. In some embodiments, the clinical laboratory tests include blood samples and/or urine samples. In some embodiments, hemoglobin, hematocrit, red blood cell count, mean corpuscular hemoglobin, mean corpuscular volume, mean corpuscular hemoglobin concentration, white blood cell count (with differential) and platelet count are measured to evaluate safety and/or efficacy of a composition. In some embodiments, albumin, alkaline phosphatase, alanine aminotransferase (ALT), amylase, aspartate aminotransferase (AST), bicarbonate, bilirubin (direct, indirect and total), calcium, chloride, creatine kinase, creatinine, y-glutamyl transferase, glucose, lactate dehydrogenase, lipase, magnesium, phosphate, potassium, protein-total, sodium, blood urea nitrogen and/or uric acid are measured to evaluate the safety and/or efficacy of a composition. In some embodiments, urine is tested for pregnancy and/or illicit drugs.

In some embodiments, a subject is monitored by monitoring the signs or symptoms of depression in a subject before, during, and/or after treatment with a composition of the present disclosure. In some embodiments, the sign or symptom of depression is measured according to a diary assessment, an assessment by clinician or caregiver, a clinical rating scale, an imaging test, or a blood of CSF test.

In some embodiments, the sign or symptom of depression in a subject is measured using a neuropsychological assessment or clinical rating scale. In some embodiments, the neuropsychological assessment or clinical rating scale is the Hamilton Depression Rating Scale, the Clinical Global Impression (CGI) Scale, the Montgomery-Asberg Depression Rating Scale (MADRS), the Beck Depression Inventory (BDI), the Zung Self-Rating Depression Scale, the Raskin Depression Rating Scale, the Inventory of Depressive Symptomatology (IDS), the Quick Inventory of Depressive Symptomatology (QIDS), the Columbia-Suicide Severity Rating Scale, or the Suicidal Ideation Attributes Scale.

In some embodiments, the sign or symptom of depression in a subject is measured using the Hamilton Depression Rating (HAM-D) scale. The HAM-D scale is a 17-item scale that measures depression severity before, during, or after treatment. The scoring is based on 17 items and generally takes 15-20 minutes to complete the interview and score the results. Eight items are scored on a 5-point scale, ranging from 0=not present to 4=severe. Nine items are scored on a 3-point scale, ranging from 0=not present to 2=severe. A score of 10-13 indicates mild depression, a score of 14-17 indicates mild to moderate depression, and a score over 17 indicates moderate to severe depression. In some embodiments, after treatment with the methods described herein, the subject's HAM-D score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Clinical Global Impression (CGI) scale. The CGI scale is a 3-item scale that measures illness severity, global improvement or change, and therapeutic response. The CGI is rated on a 7-point scale, with the severity of illness measured using a range of responses from 1 (normal) through 7 (amongst the most severely ill subjects). In some embodiments, after treatment with the methods described herein, the subject's CGI score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Montgomery-Asberg Depression Rating Scale (MADRS). The MADRS scale is a 10-item scale that measures the core symptoms of depression. Nine of the items are based upon patient report, and 1 item is on the rater's observation during the rating interview. A score of 7 to 19 indicates mild depression, 20 to 34 indicates moderate depression, and over 34 indicates severe depression. MADRS items are rated on a 0-6 continuum with 0=no abnormality and 6=severe abnormality. In some embodiments, after treatment with the methods described herein, the subject's MADRS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In some embodiments, the sign or symptom of depression in a subject is measured using the Beck Depression Inventory (BDI). The BDI is a 21-item, self-report rating inventory that measures characteristic attitudes and symptoms of depression. The items are rated on 0-3 continuum with 0=no abnormality and 3=severe abnormality. A score of 17-20 indicates borderline clinical depression, a score of 21-30 indicates moderate depression, a score of 31-40 indicates severe depression, and over 40 indicates extreme depression. In some embodiments, after treatment with the methods described herein, the subject's BDI score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Zung Self-Rating Depression Scale. The Zung Self-Rating Depression Scale is a 20-item self-report questionnaire that measures the psychological and somatic symptoms associated with depression. The questionnaire takes about 10 minutes to complete, and items are framed in terms of positive and negative statements. Each item is scored on a Likert scale ranging from 1 to 4. A total score is derived by summing the individual item scores, and ranges from 20 to 80. Most people with depression score between 50 and 69, while a score of 70 and above indicates severe depression. In some embodiments, after treatment with the methods described herein, the subject's Zung Self-Rating Depression score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Raskin Depression Rating Scale. The Raskin Depression Rating Scale measures baseline levels of depression and change in depression severity overtime. Each item is scored on a scale ranging from 1 to 5 with 1=not at all and 5=very much. A total score is derived by summing the individual item scores, and scores of 9 or greater represents moderate depression. In some embodiments, after treatment with the methods described herein, the subject's Raskin Depression Rating Scale score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Inventory of Depressive Symptomatology (IDS). The IDS is a 30-item inventory that measures depressive signs and symptoms. Each item is scored on a scale of 0 to 3 with 0=absence of pathology and 3=severe pathology. A total score is derived by summing the individual item scores; scores between 26-38 indicates mild depression, scores between 39-48 indicate moderate depression, and scores 49 or greater indicate severe depression. In some embodiments, after treatment with the methods described herein, the subject's IDS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 900%, about 95%, or about 100%, or more, compared to prior to treatment.

In some embodiments, the sign or symptom of depression in a subject is measured using the Quick Inventory of Depressive Symptomatology (QIDS). The QIDS is a 16-item inventory that measures depressive signs and symptoms. Each item is scored on a scale of 0 to 3 with 0=absence of pathology and 3=severe pathology. A total score is derived by summing the individual item scores; scores between 11-15 indicate moderate depression, scores between 16-indicate severe depression, and scores 21 or greater indicate very severe depression. In some embodiments, after treatment with the methods described herein, the subject's QIDS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 900%, about 95%, or about 100%, or more, compared to prior to treatment.

In some embodiments, the sign or symptom of depression in a subject is measured using the Young Mania Rating Scale (YMRS). The YMRS is an 11-item inventory that measures manic signs and symptoms. There are 4 items that are graded on a 0 to 8 scale, ranging from 0=absent to 8=severe. The remaining 7 items are graded on a 0 to 4 scale, ranging from 0=absent to 4=severe. A total score is derived by summing the individual item scores; scores between 9-15 indicate mild mania, scores between 16-25 indicate moderate mania, and scores 26 or greater indicate severe mania. In some embodiments, after treatment with the methods described herein, the subject's YMRS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Columbia-Suicide Severity Rating Scale (C-SSRS). The C-SSRS measures the severity of suicidal ideation and behavior. The scale contains 10 binary questions (no=0 points and yes=1 point) and each question addresses a different component of the subject's suicidal ideation severity and behavior. A subject is considered to have suicidal ideation and/or behavior if they answer “yes” to any of the 10 questions. In some embodiments, after treatment with the methods described herein, the subject's C-SSRS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using the Suicidal Ideation Attributes Scale (SIDAS). The SIDAS measures the presence and severity of suicidal thoughts. The scale contains 5 questions measured on a 10-point scale with 0=never and 10=always. Total scores are calculated as the sum of the 5 items and range from 0 to 50. Scores of 21 or greater indicate high risk of suicidal behavior. In some embodiments, after treatment with the methods described herein, the subject's SIDAS score decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression in a subject is measured using an imaging test before, during, or after treatment with the methods described herein. In some embodiments, the imaging test is a CT scan. In some embodiments, the imaging test is a functional MRI scan. In some embodiments, the functional MRI scan measures the blood oxygen level-dependent (BOLD) response as an indicator of brain activity and/or functional connectivity. In some embodiments, the BOLD response is measured in the subject at resting state, in response to emotional faces, or in response to music. In some embodiments, after treatment with the methods described herein, the BOLD response in a region of the brain increases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In some embodiments, after treatment with the methods described herein, the BOLD response in the amygdala increases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In other embodiments, after treatment with the methods described herein, the BOLD response in a region of the brain decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment. In some embodiments, after treatment with the methods described herein, the BOLD response in the amygdala decreases by between about 5% and about 100%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, or more, compared to prior to treatment.

In some embodiments, the sign or symptom of depression is measured using a marker for depression in the blood or cerebral spinal fluid. In some embodiments, the marker for depression is measured in the subject before, during, or after treatment with the methods or compositions described herein. In some embodiments, the marker for depression is red blood cell folate, serum folate, vitamin B12, plasma homocysteine, serum methylfolate, and/or testing for one or more of brain-derived neurotrophic factor (BDNF) Val66Met, bone morphogenetic protein rs41271330, and/or 5-HTTLPR polymorphisms. In some embodiments, the marker for depression decreases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% about 100%, about 110%, about 120%, about 130%, about 140%, about 150%, about 160% about 170% about 180% about 190% about 200% about 210% about 220% about 230% about 240%, about 250%, about 260%, about 270%, about 280%, about 290%, or about 300%, or more, compared to prior to treatment. In other embodiments, the marker for depression increases by between about 5% and about 300%, for example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 2200%, about 230%, about 240%, about 250%, about 260%, about 270%, about 280%, about 290%, or about 300%, or more, compared to prior to treatment.

In addition to the above, there are validated animal models of traumatic brain injury (TBI), anxiety, and/or depression. In some embodiments, the animal model of TBI is a Controlled Cortical Impact (CCI) injury model. In some embodiments, CCI models common the cortical contusion, defined as a focal destruction of brain tissue with micro hemorrhages, either with intact pia mater (contusion) or a torn pia mater (laceration). Importantly, contusions frequently enlarge markedly during the initial days post-TBI. Cortical contusions predominantly occur in the frontal and temporal regions and are often observed also at brain regions opposite to the initial impact (contre-coup contusion). The CCI model uses a pneumatic or electromagnetic impact devise to drive a rigid impactor onto the exposed intact dura, and mimics cortical tissue loss, acute subdural hematoma, axonal injury, concussion, blood-brain barrier (BBB) dysfunction and even coma. In some embodiments, CCI can be used in ferrets, rats, mice, swine and/or monkey models.

In some embodiments, the animal model of TBI is a Weight Drop TBI—Maryland Model. In some embodiments, the Weight Drop TBI—Maryland Model produces TBI by causing anterior posterior plus sagittal rotational acceleration of the brain inside the intact cranium. The animals are characterized by an absence of cortical contusions, skull fractures, prolonged apnea, and an absence of mortality, but demonstrate petechial hemorrhages and diffuse axonal injury. Neurobehavioral dysfunction, manifesting as reduced spontaneous exploration, persists for more than 1 week. In some embodiments, Weight Drop TBI—Maryland Model can be used in ferrets, rats, mice, swine and/or monkey models.

In some embodiments, the animal model of cognitive impairment associated with TBI, anxiety, and/or depression is Morris Water Maze (MWM) and/or Novel Object Recognition (NOR) assessments. Several tests may be used to support the potential cognitive benefits associated with the combination(s) (psilocybin, CBD, mushroom blend, psilocybin+CBD, and psilocybin+CBD+mushroom blend) including but not limited to MWM and NOR assessments.

Spatial learning and memory are the most studied aspects of cognition in animal TBI model studies, the Morris water maze (MWM), is among the tasks most frequently used. The MWM consists of a circular pool of water that contains an escape platform and is surrounded by visual spatial cues that the rodent can use to assist it in locating the escape platform. Measures that used to assess ability to locate the platform, including search latency, swim path, distance traveled, time spent in the platform quadrant, and search strategy/efficiency (e.g., direct and circle swims). Previous MWM studies in TBI animal models have found deficits in learning, cognitive flexibility, working memory, as well as retrograde and anterograde amnesia.

The Novel Object Recognition (NOR) test evaluates recognition memory and involves placing a rodent in an open field that contains familiar and/or novel objects. The animal will be familiarized with two objects, and following an intertrial time interval, one of these original/familiar objects will be replaced with a novel object. The novel object is typically recognized and preferred by healthy rodents, whereas rodents given TBI have short- and long-term cognitive deficits. These findings are consistent with those from human TBI studies that have reported impairments in recognition memory.

In some embodiments, the animal model of anxiety is an elevated plus maze (EPM), an open field test (OFT), and/or a forced swim test (FST). Many TBI subject experience a psychiatric illness after a TBI, with depression and anxiety the most common presentations. Depression and anxiety assessments assist in the translational value of pharmacologic agents used to treat TBI and/or TBI associated anxiety and/or depression. Many tests may be used to support the benefits associated with the combination(s) (psilocybin, CBD, mushroom blend, psilocybin+CBD, and psilocybin+CBD+mushroom blend) including but not limited to EPM, OFT and/or FST assessments.

Many tasks have been designed to measure anxiety in rodents, using the animal's natural conflicts of fear of open spaces against the desire to explore its environment to search for food. The Elevated Plus Maze (EPM) is a behavioral task assessment that uses this strategy and is used to explore anxiety-like behaviors after TBI. The EPM test is widely used to study anxiety and has been used to identify deficits in regions such as the limbic regions, hippocampus, amygdala, and dorsal raphe. The animal is placed at the junction of the four arms of the maze, two of which are closed by walls and two are open. The animal is placed facing an open arm and the time spent or number of entries in the open or closed arms of the maze is recorded. The time spent in the closed arms correlates to anxiety, since the animal lacks the drive to explore the open arm that is less safe.

The Open Field Test (OFT) assesses the animal's locomotor activity, exploratory behavior, and anxiety. The test places the animal in a square, rectangle or circular box with set spacing requirements and the animal's exploratory behavior is monitored. Two factors are known to influence anxiety-like behavior in the open field. The first is social isolation and the second is stress/aversion created by the brightly lit, unprotected, novel test environment.

Rodents exposed to the novel experimental arena typically spend greater time exploring the periphery rather than the center area. In addition to exploratory activity other anxiety behaviors such as grooming, rearing, and defecation are typically recorded.

The Forced Swim Test (FST) is one of the most frequently used behavior tasks used to assess depression behaviors. Rodents are placed in an inescapable transparent tank filled with water and their escape related mobility behavior is measured to evaluate depression. Increased time spent immobile in the tank correlates with depression traits such as despair and disengagement from stress coping.

In some embodiments, the animal model of TBI, anxiety, and/or depression is a social interactions test. Impairments in social functioning have been recognized as being among the most debilitating long-term issues for TBI patients. Social function is a key contributor to long-term quality of life. Several forms of social functioning have been explored in a range of experimental models of TBI. Social functioning assessments assist in the translational value of pharmacologic agents used to treat TBI will be evaluated in the TBI animal models in addition to healthy animals.

Many tests may be used to support the potential social function benefits associated with the combination(s) (psilocybin, CBD, mushroom blend, psilocybin+CBD, and psilocybin+CBD+mushroom blend) including but not limited to the social interactions test.

The Social Interactions Test includes the introduction of an unfamiliar animal to the test animal as a social stimulus and the degree of social interest is quantified at various timepoints.

In some embodiments, the animal model of TBI, anxiety and/or depression is a motor test. TBI may affect the mechanical aspects of motor behavior, such as range of movement and muscle strength and interfere with postural and phasic motor movements. Balance disturbances are one of the most reported deficits after TBI. Balance requires complex interactions and integration of many neural and musculoskeletal systems. Many tests may be used to support the potential neuromotor effects associated with the combination(s) (psilocybin, CBD, mushroom blend, psilocybin+CBD, and psilocybin+CBD+mushroom blend) including but not limited to the beam walking task.

Beam walking tasks are specifically advantageous for testing motor impairments in animals given that they are not subject to practice effects and do not have any memory or learning components. Animals are evaluated for beam-balance latency by placing the animal on a narrow beam and measuring the duration of the time that the animal can remain on the beam.

In some embodiments, the animal model of TBI, anxiety and/or depression is a pain test. Pain syndromes, such as headaches and musculoskeletal pain, are common and debilitating consequences of TBI. When referring to animal models of pain, including headache, testing is highly dependent upon measurements of nociception. Both acute and chronic pain assessments assist in the translational value pharmacologic agents used to treat TBI. Many tests may be used to support the potential pain relief benefits associated with the combination(s) (psilocybin, CBD, mushroom blend, psilocybin+CBD, and psilocybin+CBD+mushroom blend) including but not limited to the von Frey task.

The von Frey task is a measure of mechanical activation and is a reliable indicator of pain tolerance. The test will involve gradual increases in the size of filaments that poke the center of the rat's hind paw, with larger filament size being indicative of higher mechanical nociceptive (or pain) thresholds.

Additional animal assessments to evaluate TBI, anxiety and/or depression and/or the effect of an intervention on TBI, anxiety, and/or depression, include but are not limited to transection model, micro-transection model, compression model, hydrodynamic model, cell-stretch model, chemical injury model (e.g., glutamate or hydrogen peroxide), brain on a chip system, and/or organotypic cultures.

### Kits

The present disclosure contemplates kits for the treatment of anxiety, depression, stress, post-concussion syndrome, traumatic brain injury, post-traumatic headache, progressive headache, suicidal ideation, and/or symptoms thereof. In some embodiments, the kit comprises a composition of the present disclosure. In some embodiments, the composition comprises psilocybin or a derivative thereof, and/or cannabidiol (CBD), and/or a serotonergic activity modulator, and optionally a mushroom blend, or combinations thereof. The kit can include instructions for a treatment regimen. The instructions provided in a kit according to the present disclosure may be directed to suitable dosages and administration frequencies. The kit may comprise safety information relating to components of compositions contained within the kit. The kit may contain a survey for assessing safety and/or efficacy of the composition(s) or information directing a subject to a website or smartphone/tablet application for assessing safety and/or efficacy of the compositions(s). In some embodiments, the kit may contain separate compartments or containers for storing different dosage forms and/or compositions to be administered to a subject. The containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, an agent of the disclosure is provided together with instructions for administering the agent to a subject having suffered a traumatic brain injury or having post-concussion syndrome, post-traumatic headache, progressive headache, or symptoms thereof. The instructions will generally include information about the use of the composition(s) for the treatment of the injury or syndrome. In other embodiments, the instructions include at least one of the following: description of the therapeutic agent; dosage schedule and administration for treatment; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container. The instructions may be provided on a portable electronic storage medium (e.g., a DVD, a CD, or a USB drive) or the instructions may be stored on a remote server and accessible through the use of a smart phone or tablet application or a web browser.

The following examples are put forth so as to provide those of ordinary skill in the art with a complete disclosure and description of how to make and use the assay, screening, and therapeutic methods of the disclosure, and are not intended to limit the scope of what the inventors regard as their disclosure.

### EXAMPLES

### Example 1: Prepared Compositions

The compositions indicated in Tables 1 and 2 were prepared for administration to a subject. Table 3 provides dosage amounts for combination products falling within the disclosure.

TABLE 1
Compositions, where “x” indicates presence of a component in the indicated composition.

| | Composition | Composition | Composition | Composition | Composition | Composition |
|---|---|---|---|---|---|---|
| Component | 1 | 2 | 3 | 4 | 5 | 6 |
| Cannabidiol (CBD) | 50 | 150 | 25 | 25 | 25 | 25 |
| | mg/capsule | mg/oz | mg/ml | mg/ml | mg/capsule | mg/capsule |
| Solvent-Free Full Spectrum Hemp | x | | | | | |
| Extract | | | | | | |
| Full Spectrum Organic Hemp Extract | | | | | | x |
| Organic Full Spectrum Hemp Oil | | | x | | | |
| Broad Spectrum Organic Hemp Oil | | | | x | | |
| Broad Spectrum Organic Hemp Extract | | | | | x | |
| Broad Spectrum Hemp Extract | | | | | | |
| Organic Tapioca Starch | x | | | | x | x |
| Vegan Capsule | x | | | | x | x |
| Hemp Oil | | x | | | | |
| Coconut Oil | | x | | | | |
| Mango Butter | | x | | | | |
| Cocoa Butter | | x | | | | |
| Beeswax | | x | | | | |
| Natural Flavors (peppermint-vanilla) | | | x | x | | |
| Sorbitan Monooleate | | | x | x | | |
| Polysorbate 80 | | | x | x | | |
| Olive Oil | | | x | x | x | x |
| Water | | | x | x | | |
| Dextrin | | | | | x | x |
| Gum Arabic | | | | | x | x |
| Serving size (daily or per | 1 capsule | | 1 ml | 1 ml | 1 capsule | 1 capsule |
| administration) | | | | | | |

TABLE 2
Compositions, where “x” indicates presence of a component in the indicated composition.

| | Composition | Composition | Composition | Composition | Composition | Composition |
|---|---|---|---|---|---|---|
| Component | 7 | 8 | 9 | 10 | 11 | 12 |
| Cannabidiol (CBD) | 25 | 10 | 10 | | | 50 |
| | mg/ml | mg/capsule | mg/capsule | | | mg/capsule |
| Solvent-Free Full Spectrum Hemp | | x | | | | |
| Extract | | | | | | |
| Emulsified Full Spectrum Hemp Oil | x | | | | | |
| Broad Spectrum Hemp Extract | | | x | | | x |
| Organic Tapioca Starch | | | | | | x |
| Vegan Capsule | | x | x | x | x | x |
| Natural Flavors (peppermint-vanilla) | x | | | | | |
| Sorbitan Monooleate | x | | | | | |
| Polysorbate 80 | x | | | | | |
| Olive Oil | x | | | | | |
| Water | x | | | | | |
| Turmeric | | 70 | 40 | | | |
| | | mg/capsule | mg/capsule | | | |
| Piperine | | x | | | | |
| Dried Mushroom Blend: | | 400 | 400 | 523.33 | | |
| | | mg/capsule | mg/capsule | mg/capsule | | |
| Agarikon (Laricifomes officinalis) | | x | x | x | | |
| Chaga (Inonotus obliquus) | | x | x | x | | |
| Cordyceps (Cordyceps sp.) | | x | x | x | | |
| Lion's Mane (Hericium erinaceus) | | x | x | x | 240 | |
| | | | | | mg/capsule | |
| Reishi (Ganoderma lingzhi) | | x | x | x | | |
| Turkey Tail (Trametes versicolor) | | x | x | x | | |
| Mesima (Phellinus linteus) | | | | x | | |
| Maitake (Grifola frondosa) | | | | x | | |
| Shiitake (Lentinula edodes) | | | | x | | |
| Blazei (Agaricus subrufescens) | | | | x | | |
| Poria (Wolfiporia extensa) | | | | x | | |
| Suehirotake (Schizophyllum | | | | x | | |
| commune) | | | | | | |
| Oyster Mushroom (Pleurotus | | | | x | | |
| ostreatus) | | | | | | |
| True Tinder Polypore (Fomes | | | | x | | |
| fomentarius) | | | | | | |
| Niacin | | | | | 5 | |
| | | | | | mg/capsule | |
| Flax Seed Extract | | | | | 166.67 | |
| | | | | | mg/capsule | |
| Serving size (daily or per | 1 | 3 | 3 | 3 | 3 | 1 |
| administration) | ml | capsules | capsules | capsules | capsules | capsule |

TABLE 3
Compositions, where “x” indicates presence of a component in the indicated composition.

| | Composition | Composition | Composition | Composition | Composition |
|---|---|---|---|---|---|
| Component | 13 | 14 | 15 | 16 | 17 |
| Cannabidiol (CBD) - synthetic | 300 mg | 300 mg | 600 mg | 600 mg | |
| Psilocybin - synthetic | 2.5 mg | 3.0 mg | 2.5 mg | 3.0 mg | 25 mg |
| Mushroom blend | 0.5 mg | 0.5 mg | 0.5 mg | 0.5 mg | 0.5 mg |
| Capsule, tab, lozenge, | x | x | x | x | x |
| solution, sublingual strip, | | | | | |
| gummy, transdermal patch | | | | | |
| Per administration | x | x | x | x | x |
| | Composition | Composition | Composition | Composition | Composition |
| Component | 18 | 19 | 20 | 21 | 22 |
| Cannabidiol (CBD) - synthetic | | 300 mg | 600 mg | 300 mg | 600 mg |
| Psilocybin - synthetic | 30 mg | 2.5 mg | 3.0 mg | 3.0 mg | 2.5 mg |
| Mushroom blend | 0.5 mg | | | 0.5 mg | 0.5 mg |
| Capsule, tab, lozenge, | x | x | x | x | x |
| solution, sublingual strip, | | | | | |
| gummy, transdermal patch | | | | | |
| Per administration | x | x | x | x | x |

### Example 2: Exemplary Method for Treating TBI

A human subject with traumatic brain injury is orally administered between 25 mg to 30 mg synthetic psilocybin as a loading dose. Fourteen days later, the subject is orally administered a maintenance composition of synthetic psilocybin (0.25-0.3 mg) and synthetic cannabidiol (300 mg-600 mg) via a weekly protocol consisting of daily administration (after an overnight fast) of the composition for 5 consecutive days followed by a drug holiday (i.e., no administration) for 2 consecutive days.

### Example 3: Exemplary Method for Treating TBI

A human subject with traumatic brain injury is orally administered a between 25 mg to 30 mg synthetic psilocybin as a loading dose. Seven days later, the subject is orally administered a maintenance composition of synthetic psilocybin (0.25-0.3 mg), synthetic cannabidiol (300 mg-600 mg), and 0.5 mg of a mushroom blend (comprising Hericium erinaceus and Ganoderma lingzhi) via a weekly protocol consisting of daily administration (after an overnight fast) of the composition for 5 consecutive days followed by a drug holiday (i.e., no administration) for 2 consecutive days.

### Example 4: Clinical Example—Subject With Traumatic Brain Injury

A human subject with traumatic brain injury is orally administered a single 5.6-gram dose of dried mushrooms containing psilocybin as a loading dose. (20 capsules at 0.28 mg per capsule). Fourteen days later, the subject is orally administered a mushroom blend, psilocybin (provided as Psilocybe cubensis), and cannabidiol via a weekly protocol consisting of daily administration (after an overnight fast) of the composition for 5 consecutive days followed by a drug holiday (i.e., no administration) for 2 consecutive days.

The composition is provided as:
three capsules, each containing cannabidiol (10 mg), Hericium erinaceus (78.3 mg), Laricifomes officinalis (78.3 mg), Cordyceps sp. (78.3 mg), Ganoderma lingzhi (78.3 mg), Trametes versicolor (78.3 mg), and Inonotus obliquus (78.3 mg); and
one capsule containing Inonotus obliquus (34 mg), Agaricus subrufescens (20.4 mg), Laricifomes officinalis (17 mg), Trametes versicolor (20.4 mg), Ganoderma lingzhi (34 mg), Schizophyllum commune (17 mg), Hericium erinaceus (34 mg), Wolfiporia extensa (20.4 mg), Cordyceps sp. (34 mg), Fomes fomentarius (17 mg), Pleurotus ostreatus (17 mg), Phellinus linteus (34 mg), Grifola frondosa (20.4 mg), and Lentinula edodes (20.4 mg), and Psilocybe cubensis (100 mg).

The cannabidiol within the capsules described above is full-spectrum cannabidiol in the form of dry sieved hemp trichomes, also known as kief. The kief is extracted from hemp (grown under organic practices) using no solvents and a dry tumbling process. The kief is then activated by decarboxylation and freeze-dried to powder prior to encapsulation. Thus, the cannabidiol is encapsulated, freeze-dried decarboxylated dry sift kief.

The mushrooms comprised in the mushroom blend are grown under organic practices and steam activated after harvest to break down parts of the mushroom fruiting body and/or mycelium, before being freeze-dried to powder prior to encapsulation.

### Example 5: A Treatment Protocol of Psilocybin, CBD, and a 6 Mushroom Blend does not Exacerbate Depressive Symptoms

An investigation was performed on the combined effect of a novel combination of psilocybin, CBD and a 6-mushroom blend (agarikon, chaga, cordyceps, lion's mane, reishi, and turkey tail; 6 MB) on mouse locomotor activity. Mouse somnolence- and catatonia-related behaviors were assessed using the validated open field test. The intended use is for the compositions of the disclosure is for the treatment of TBI, including TBI associated depression, and any somnolence- or catatonia-related behaviors could exacerbate the underlying condition.

Psilocybin was dosed at 0.3 mg/kg in all groups, whereas the CBD and 6 MB were tested at 3 different doses (120:5640 mg/kg, 60:2820, and 30:1410 mg/kg of CBD and the 6 MB, respectfully) that approximate doses to be tested clinically.

Animals and Protocol: Young mice (BALB/c, <9 weeks of age) were ordered by Charles River Laboratories and the protocol carried out at a Charles River facility in Finland. After arriving at the Testing Facility, animals were acclimated for seven (7) days prior to the beginning of the in-life phase. Plasma bile acid analysis was performed to exclude animals with abnormally high bile acid concentration (>20 μmol/L) which is a surrogate marker of portosystemic liver shunt which could confound interpretation of results.

On testing day, mice were brought to the experimental room for at least a 1 hour acclimation period prior to testing. Activity chambers were equipped with infrared (IR) beams and mice were placed in the center of the chamber and their behavior was recorded for 30 min in 5-min bins. Quantitative analysis of behaviors in the open field test was performed on the following six dependent measures: total locomotion, locomotion in the center of the open field, rearing rate in the center, total rearing frequency, resting time and velocity. Animals were tested at low-stress conditions with light intensity lowered to approximately 10-30 lux of red light. Open field tests were conducted at 120 min and 24 hours after dosing.

Analysis: Simple comparisons between two groups were performed using unpaired Welch's t-test, or, if the assumption of normality or log normality was not met, by the Mann-Whitney U-test. Comparison involving three or more groups was performed using one-way ANOVA followed by Dunnett's multiple comparisons test (treatment vs. control). In case of significantly different SDs detected by Brown-Forsythe test, the assumption of equal SDs was rejected and Welch's ANOVA followed by Dunnett's T3 multiple comparison test was used. The non-parametric counterpart is the Kruskal-Wallis test followed by Dunn's multiple comparison test.

Drug preparation and administration: All drugs were diluted in a vehicle solution containing 0.1% Tween 80 and 0.5% HPMC. Vehicle or combined formulation of test articles (Psilocybin [Toronto Research Chemicals], CBD [Boulder Hemp] and 6 MB [Boulder Hemp]) was performed by investigators blinded to the treatment. The vehicle or combined formulation (described above) volume for each animal was based on the body weight and delivered orally at a volume of 20 mL/kg.

Results and Conclusion: While some effects of the combination were observed at the 2-hour testing timepoint compared to vehicle (decreased time spent in the middle, vertical counts and velocity) there was no appreciable or consistent effect of the combination on ambulatory distance or resting time.

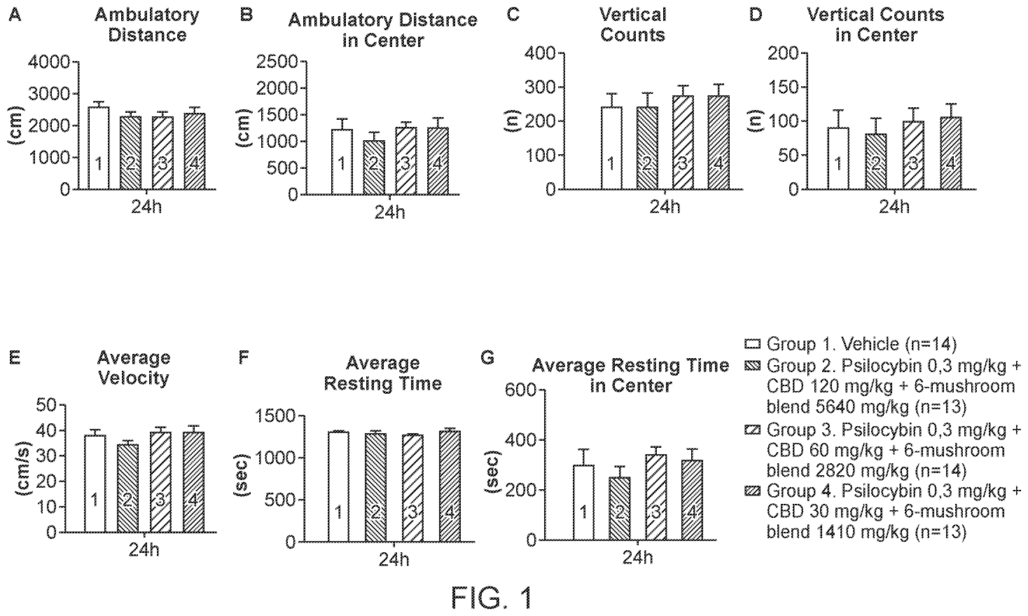

The 24-hour post-dosing timepoint provides insight into the potential for sustained somnolence and catatonia-related behaviors or next-day residual effects. Reassuringly, no statistically significant findings between the vehicle nor any of the active treatment arms were observed at the 24-hour timepoint following dosing for any of the dependent measures including total locomotion, locomotion in the center of the open field, rearing rate in the center, total rearing frequency, resting time and velocity (FIG. 1). This indicates that the tested combination of psilocybin, CBD, and a 6 MB does not induce appreciable somnolence-nor catatonia-related behaviors and is therefore unlikely to potentiate the symptoms of depression. This data can be interpreted to indicate that the tested compositions would not exacerbate TBI associated depression in clinical patients. FIG. 1 has a legend in the lower right hand corner identifying Groups 1 through 4, wherein each of FIG. 1A through FIG. 1G sub-panels charts Group 1, Group 2, Group 3, and Group 4 from left to right within each sub-panel.

### Example 6: A Treatment Protocol of Psilocybin, CBD, and a 6 Mushroom Blend Attenuates Depression and Anxiety Symptoms in Mice

Depression is common after TBI. About half of all people with TBI are affected by depression within the first year following injury. Even more (nearly two-thirds) of those with TBI are affected by depression within seven years after injury. In the general population, the rate of depression is significantly lower, affecting less than one person in 10 over a one-year period. Moreover, more than half of the people with TBI who are depressed also have significant anxiety. There are few treatment options available for individuals with TBI and concurrent conditions such as depression and anxiety and as such these individuals often suffer from severe morbidity. The objective of this study was to investigate the effects of a novel protocol of a psilocybin loading dose followed by combination product containing a unique combination of psilocybin, CBD, and a 6-mushroom blend (agarikon, chaga, cordyceps, lion's mane, reishi and turkey tail; 6 MB) on validated animal models of anxiety and depression using the Elevated Maze Test and the Tail Suspension Test.

Animals and Protocol: Young mice (BALB/c, <9 weeks of age) were ordered by Charles River Laboratories and the protocol carried out at a Charles River facility in Finland. After arriving at the Testing Facility, animals were acclimated for seven (7) days prior to the beginning of the in-life phase. As relevant, single loading doses of psilocybin (5 mg/kg) were given on Day 1. A combination dose of psilocybin, CBD and 6 MB were given for 5 consecutive days (Days 16-Day 20). The mice were assessed via elevated plus maze (EPM) for anxiety at 3 time points on days 14, 21 and 28. EPM is widely used behavioral assay for measuring anxiety-like behavior in rodents. The test is based on the natural aversion of mice for open and elevated areas, as well as on their natural spontaneous exploratory behavior in novel environments. The mice were also assessed via tail suspension test (TST) for depression on days 15, 22 and 29. The TST is commonly used in the screening of potential antidepressant drugs. Animals subjected to the short-term, inescapable stress will develop an immobile posture which is considered as a modeling state of “behavioral despair.”

The elevated plus maze consists of two opposing open arms and two enclosed arms that extend from a central platform, elevated above the floor. Animals were transferred from their home cage to the center of a plus maze facing an open arm and monitored for 5 min. After the 5 min period, the animal was transferred back to its home cage. The number of entries to closed and open arms as well as time spent in open and closed arms were measured. The relative portion of open/(open+closed) time/visits was used as an index for anxiety-like behavior. Animals were tested during light cycle (7:00-20:00). Prior to any procedure, all animals were allowed a 60-min habituation to a test room.

The tail suspension test was performed with animals suspended with surgical tape from the tails and total immobility time was recorded during 6 min testing period. Immobility was defined as not having limb or body movement (except breathing). Immobility was scored using video tracking and movement analysis (Noldus Ethovision XT). Animals were tested during light cycle (7:00-20:00). Prior to any procedure, all animals were allowed a 60-min habituation to a test room.

Drug preparation and administration: A psilocybin loading dose of 5 mg/kg was administered 2-weeks prior to behavioral testing and a maintenance dose of a combination formulation (comprising psilocybin, CBD, and a 6 mushroom blend [6 MB]) was administered five (5) consecutive days prior to testing. The microdose or maintenance dose of psilocybin was 0.3 mg/kg and given in combination with CBD and 6 MB tested at 3 different doses (120:5670 mg/kg, 60:2820, and 30:1410 mg/kg of CBD and the 6 MB, respectively). These doses were chosen to approximate doses to be tested clinically in humans. The combination formulation was dosed orally (p.o, 10 ml/kg). All drugs were diluted in a vehicle solution containing 0.1% Tween 80 and 0.5% HPMC. Vehicle, combination formulation (Psilocybin [Toronto Research Chemicals], CBD [Boulder Hemp] and 6 MB [Boulder Hemp]) was performed by investigators blinded to the treatment. The dose volume for each animal was based on the body weight.

Analysis: Simple comparisons between two groups will be performed using unpaired Welch's t-test, or, when the assumption of normality or log normality is not met, by the Mann-Whitney U-test. Comparison involving three or more groups will be performed using one-way ANOVA followed by Dunnett's multiple comparisons test (treatment vs. control). In case of significantly different SDs detected by Brown-Forsythe test, the assumption of equal SDs is rejected and Welch's ANOVA followed by Dunnett's T3 multiple comparison test will be used. The non-parametric counterpart is the Kruskal-Wallis test followed by Dunn's multiple comparison test.

Results and Conclusion: Both a loading dose of psilocybin, as well as a combination of loading dose of psilocybin, followed by low dose psilocybin, CBD, and a 6 mushroom blend decreased anxiety and depression in mouse models.

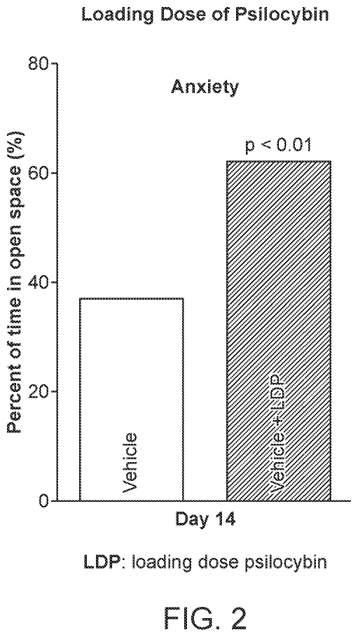

The mice receiving a loading dose of psilocybin (5 mg/kg) spent more time exploring the open arms of the EPM at Day 14, than mice receiving a vehicle control (FIG. 2). As mice (a prey species) do not like to spend time in open spaces, the greater the amount of time the mice spent in open spaces after administration of the loading dose can be interpreted to mean that the mice were demonstrating less anxiety.

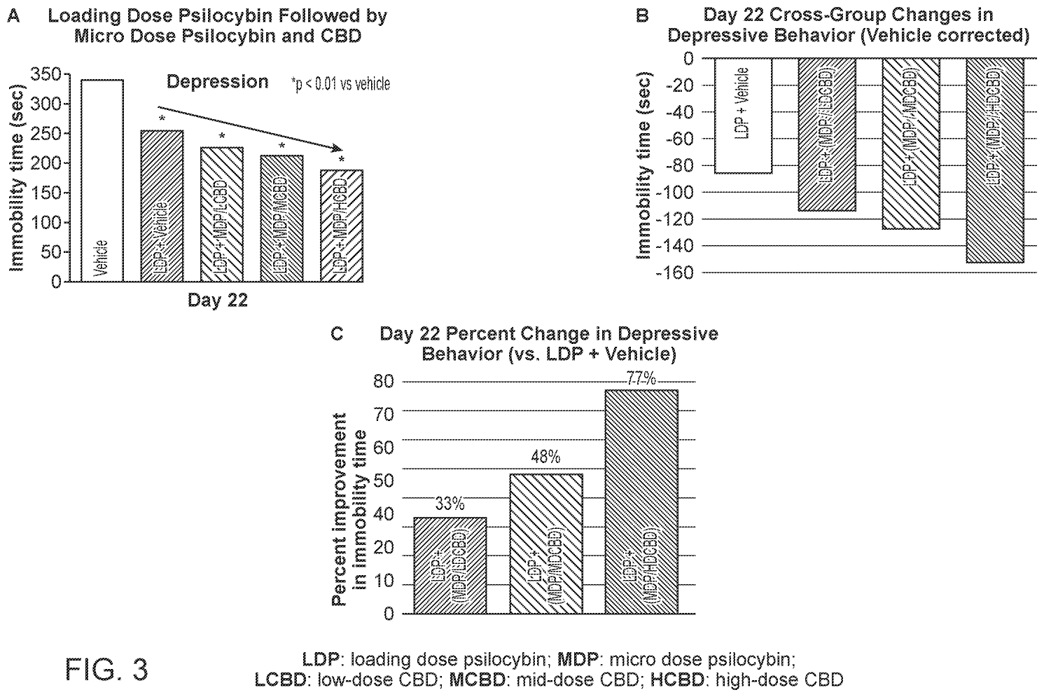

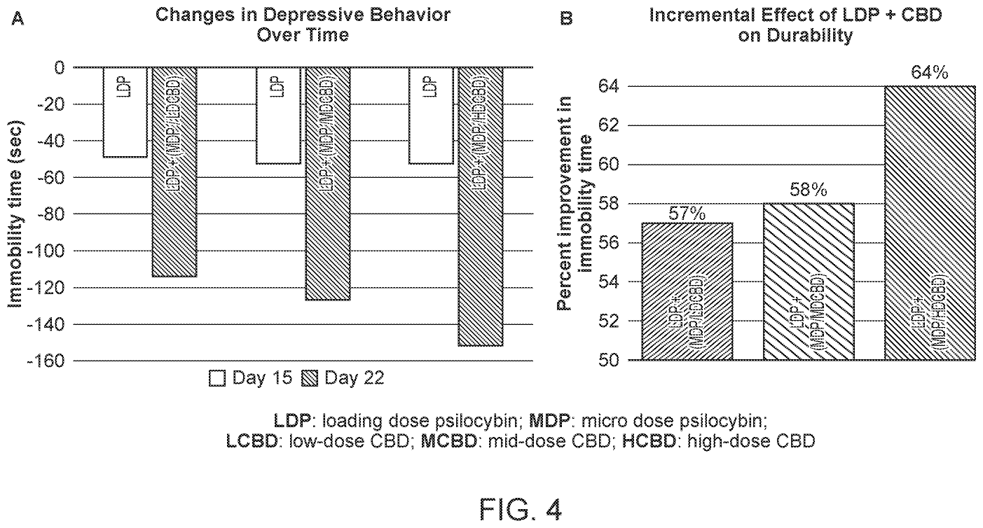

The immobile posture time observed in the TST was decreased in animals receiving psilocybin loading dose at Day 15 (FIG. 4A). By Day 22, all groups receiving a loading dose of psilocybin had significantly lower immobility time (FIG. 4A). This shows that additional gains can be made (amount of time spent immobile can be decreased) by adding a maintenance dose of psilocybin, CBD, and a 6 mushroom blend. Also when comparing across groups, a dose-dependent effect was observed in groups receiving a loading dose of psilocybin followed by the high-dose, mid-dose, and low-dose (120:5670 mg/kg, 60:2820, and 30:1410 mg/kg of CBD and 6 MB, respectively) CBD/6 MB combinations (FIG. 3A). Specifically, a trend can be seen where, while a significant decrease in the time spent immobile was observed three weeks after the administration of a 5 mg/kg loading dose of psilocybin, further reductions in time spent immobile were observed when a 0.3 mg/kg microdose of psilocybin as well as increasing amounts of a maintenance CBD and a 6 mushroom blend dose was administered after the initial loading dose of psilocybin (FIGS. 3A-C). The group with the highest CBD/6 MB dose (HCBD/HDCBD) had the lowest immobility time (45% reduction from vehicle) and lowest CBD/6 MB dose group (LCBD/LDCBD) had a greater immobility time (34% reduction from vehicle), supporting the efficacy of the combination of a loading dose psilocybin followed by a maintenance dose of psilocybin+CBD+6 MB combination on depression-like tendencies (FIG. 3A). The less time spent immobile is considered to be less depressive behavior.

It is unexpected that when a loading dose of psilocybin, followed by a 5-day dosage regimen of sub-perceptual psilocybin (˜10× lower than the loading dose of psilocybin) combined with CBD and a 6 mushroom blend, that not only were the initial improvements in antidepressive behavior maintained but in fact appeared to show a pronounced dose-dependent improvement based on the CBD component of the combination regimen (FIG. 4A).

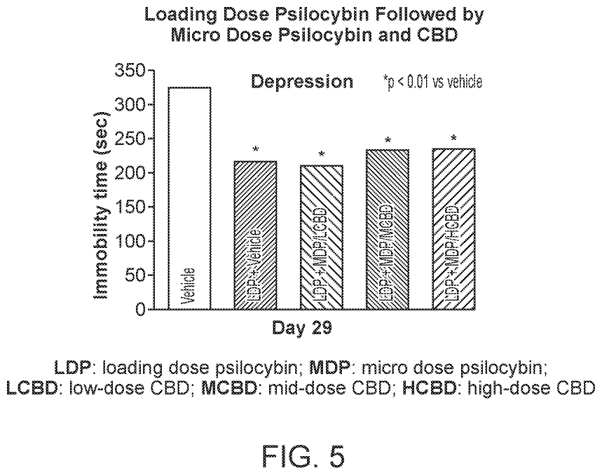

At Day 29, TST showed continued benefit related to “behavioral despair” or depression-like tendencies (˜30-35% reduction from vehicle) (FIG. 5). These data suggest a fundamental change in brain plasticity related to the psilocybin changing the threshold for depressive behaviors, which is enhanced by a maintenance regimen of low-dose psilocybin+CBD+6 MB.

In the tail suspension test, significant time spent immobile can model symptoms of depression, and a reduction of time spent immobile can indicate anti-depressive effects of the administered composition/treatment. This data can be interpreted to indicate that the protocol of a loading dose of psilocybin, followed by micro- or maintenance doses of psilocybin and a CBD/6 mushroom blend decrease depressive symptoms in mice.

### Example 7: A Treatment Protocol of Psilocybin and Imipramine Attenuates Depression Symptoms in Mice

Animals and Protocol: Young mice (BALB/c, <9 weeks of age) were ordered by Charles River Laboratories and the protocol carried out at a Charles River facility in Finland. After arriving at the Testing Facility, animals were acclimated for seven (7) days prior to the beginning of the in-life phase. As relevant, single loading doses of psilocybin (5 mg/kg) were given on Day 1. Imipraime (15 mg/kg) was given 1 hour before tail suspension testing on Day 22. TST is commonly used in the screening of potential antidepressant drugs. Animals subjected to the short-term, inescapable stress will develop an immobile posture which is considered as a modeling state of “behavioral despair.” Antidepressants generally reduce the time spent immobile. For positive control, one group was given imipramine which is known to reduce immobility in TST in stressed mice.

Tail suspension test was performed with animals suspended with surgical tape from the tails and total immobility time was recorded during 6 min testing period. Immobility was defined as not having limb or body movement (except breathing). Immobility was scored using video tracking and movement analysis (Noldus Ethovision XT). Animals were tested during light cycle (7:00-20:00). Prior to any procedure, all animals were allowed a 60-min habituation to a test room.

Analysis: Simple comparisons between two groups were measured through inferential means.

Drug preparation and administration: All drugs were diluted in a vehicle solution containing 0.1% Tween 80 and 0.5% HPMC. Vehicle or imipramine (CRL Finland) was performed by investigators blinded to the treatment. The dose volume for each animal was based on the body weight. Psilocybin loading dose was given 3-weeks prior to behavioral testing and imipramine was given 1-hour before testing on each testing day via Intraperitoneal injection (i.p, 5 ml/kg).

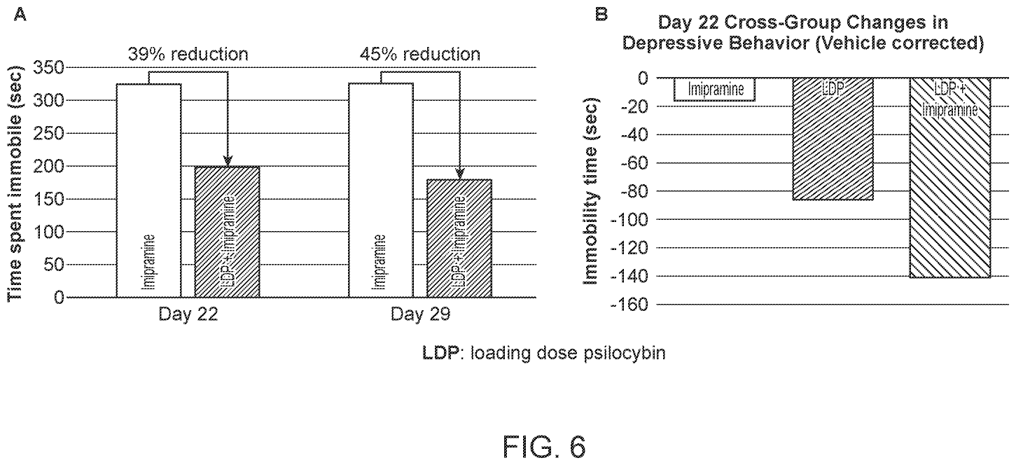

Results and Conclusion: At Day 22, there was an unexpectedly greater reduction in depression-like behaviors in the imipramine treatment group that received the 5 mg/kg loading dose of psilocybin compared to the group that only received imipramine on Day 22 (FIG. 6B). At Day 22, the group which received a loading dose of psilocybin at Day 1 spent 39% less time immobile than the group which only received imipramine (15 mg/kg) one hour before TST (FIG. 6A). At Day 29, the group which received a loading dose of psilocybin at Day 1 spent 45% less time immobile than the group which only received imipramine (15 mg/kg) one hour before TST (FIG. 6A). These results demonstrate that psilocybin can potentiate the effects of antidepressant medications. When the vehicle corrected analysis is considered, a synergy is observed in the psilocybin+imipramine treatment condition, compared to individual treatment arms (FIG. 6B). While all three treatments demonstrated an anti-depressant effect compared to vehicle (expressed as “0” in FIG. 6B), the loading dose+imipramine group spent the least amount of time immobile in the TST at Day 22. LDP=loading dose psilocybin (5 mg/kg).

### OTHER EMBODIMENTS

From the foregoing description, it will be apparent that variations and modifications may be made to the disclosure described herein to adapt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for reducing a symptom of traumatic brain injury or post-concussion syndrome in a subject, the method comprising:
(i) administering to the subject at least one loading dose comprising between about 5 mg and 30 mg psilocybin, inclusive; and
(ii) subsequently administering to the subject at least one maintenance dose comprising between about 0.25 mg to about 0.3 mg psilocybin, inclusive, wherein the subject has been suspected or diagnosed as having traumatic brain injury or post-concussion syndrome.

2. The method of claim 1, wherein the symptom of traumatic brain injury or post-concussion syndrome comprises anxiety, depression, suicide ideation, stress, post-traumatic stress disorder, post-traumatic headache, progressive headache, feelings of dizziness, nausea, vomiting, noise sensitivity, being easily upset by loud noise, sleep disturbance, fatigue or tiring more easily, irritability, insomnia, nervousness, frustration or impatience, forgetfulness or poor memory, poor concentration, taking longer to think, blurred vision, light sensitivity, double vision, restlessness, insomnia, ringing in the ears, blurry vision, decrease in taste and/or smell, and various combinations thereof.

3. The method of any of claims 1-2, wherein the at least one maintenance dose further comprises between about 100 mg and about 600 mg CBD, inclusive.

4. The method of any of claims 1-3, wherein the symptom of traumatic brain injury or post-concussion syndrome comprises anxiety and/or depression.

5. The method of any of claims 1-4 wherein the loading dose comprises about 10 mg psilocybin.

6. The method of any of claims 1-5, wherein the loading dose comprises about 25 mg psilocybin.

7. The method of any of claims 1-6, wherein the loading dose comprises about 30 mg psilocybin.

8. The method of any of claims 1-7, wherein the psilocybin of the loading dose and/or the maintenance dose comprises synthetic psilocybin.

9. The method of any of claims 1-8, wherein the psilocybin of the loading dose comprises synthetic psilocybin.

10. The method of any of claims 1-9, wherein the psilocybin of the maintenance dose comprises synthetic psilocybin.

11. The method of any of claims 1-10, wherein the synthetic psilocybin is crystalline psilocybin.

12. The method of any of claims 1-11, wherein the synthetic psilocybin is amorphous psilocybin.

13. The method of any of claims 1-12, wherein the psilocybin is isolated from the mushroom Psilocybe cubensis.

14. The method of any of claims 3-13, wherein the CBD comprises synthetic CBD.

15. The method of any of claims 3-14, wherein the CBD comprises full-spectrum CBD.

16. The method of any of claims 1-15, wherein the at least one maintenance dose further comprises a composition comprising at least one, at least two, at least three, at least four, at least five, or at least six mushrooms.

17. The method of any of claims 1-16, wherein the mushroom composition comprising at least one, at least two, at least three, at least four, at least five, or at least six dried mushrooms comprises between about 0.01 g and about 0.5 g of the mushroom composition.

18. The method of any of claims 16-17, wherein the mushroom composition comprises Hericium erinaceus and Ganoderma lingzhi.

19. The method of any of claims 16-18, wherein the mushroom composition comprises Laricifomes officinalis, Inonotus obliquus, Cordyceps sp., Hericium erinaceus, Ganoderma lingzhi, Trametes versicolor.

20. The method of any of claims 1-19, wherein the loading dose is administered prior to an initial maintenance dose by at least seven days.

21. The method of any of claims 1-20, wherein two or more loading doses are administered over a 6 month time period.

22. The method of any of claims 1-21, wherein the maintenance dose is administered every day.

23. The method of any of claims 1-22, wherein the maintenance dose is not administered every day.

24. The method of any of claims 1-23, wherein the maintenance dose is administered according to a predetermined schedule.

25. The method of any of claims 1-24, wherein the maintenance dose is administered for five out of every seven days.

26. The method of any of claims 1-25, wherein the maintenance dose is formulated in a single dosage form.

27. The method of any of claims 1-26, wherein the maintenance dose is formulated in multiple dosage forms.

28. A composition for treatment of traumatic brain injury or post-concussion syndrome, the composition comprising: psilocybin, and optionally a dried mushroom blend, wherein the dried mushroom blend comprises at least two mushrooms selected from the group consisting of Laricifomes officinalis, Inonotus obliquus, Cordyceps militaris, Hericium erinaceus, Gandoderma lingzhi, and Trametes versicolor.

29. The composition of claim 28, further comprising mushrooms belonging to a genus selected from the group consisting of: Agaricus, Schizophyllum, Wolfiporia, Fomes, Pleurotus, Phellinus, Grifola, and Lentinula.

30. The composition of claim 28, wherein the psilocybin and optionally the dried mushroom blend are formulated together or separately.

31. The composition of claim 28, wherein the composition further comprises a cannabinoid, niacin, piperine, turmeric, and/or an omega fatty acid.

32. The composition of claim 28, wherein the composition further comprises a serotonin reuptake inhibitor (SSRI), folic acid, St. John's Wart, dried Codiaeum variegatum, Codiaeum variegatum extract, fish oil, flax seed oil, or combinations thereof.

33. A method for reducing a symptom of traumatic brain injury or post-concussion syndrome in a subject, the method comprising:
(i) administering to the subject one or more loading dose(s) of at least about 1 mg psilocybin; and
(ii) subsequently administering to the subject one or more maintenance dose(s) comprising psilocybin and a dried mushroom blend, wherein the dried mushroom blend comprises at least two mushrooms selected from the group consisting of Laricifomes officinalis, Inonotus obliquus, Cordyceps militaris, Hericium erinaceus, Ganoderma lingzhi, and Trametes versicolor, wherein the maintenance dose comprises less than about 1 mg psilocybin, and wherein the subject has had a traumatic brain injury or has post-concussion syndrome.

34. The method of claim 33, wherein the psilocybin is purified and/or synthetic psilocybin.

35. The method of claim 33, wherein the loading dose comprises about 1 mg to about 50 mg of purified and/or synthetic psilocybin.

36. The method of claim 33, further comprising administering to the subject a subsequent loading dose, wherein the subsequent loading dose comprises from about 1 mg to about 50 mg psilocybin.

37. The method of claim 36, wherein the subsequent loading dose is separated from the most recently administered loading dose by at least about 1 month.

38. The method of claim 33, wherein the maintenance dose is administered about daily.

39. The method of claim 33, wherein the maintenance dose is administered for about 3-5 consecutive days followed by no administration for about 1-3 consecutive days.

40. The method of claim 33, wherein the maintenance dose is administered daily followed by no administration for 2 consecutive days.

41. The method of claim 33, wherein the maintenance dose further comprises turmeric and piperine.

42. The method of claim 33, wherein the maintenance dose further comprises cannabidiol.

43. The method of claim 33, wherein the maintenance dose comprises from about 0.05 to about 1 mg psilocybin.

44. The method of claim 33, wherein the maintenance dose comprises from about 5 mg to about 100 mg cannabidiol.

45. The method of claim 33, wherein the dried mushroom blend comprises:
from about 1 wt % to about 60 wt % by dry weight of Inonotus obliquus,
from about 0.3 wt % to about 8 wt % by dry weight of Agaricus subrufescens,
from about 3 wt % to about 60 wt % by dry weight of Laricifomes officinalis,
from about 3 wt % to about 60 wt % by dry weight of Trametes versicolor,
from about 3 wt % to about 60 wt % by dry weight of Ganoderma lingzhi,
from about 0.3 wt % to about 8 wt % by dry weight of Schizophyllum commune,
from about 3 wt % to about 60 wt % by dry weight of Hericium erinaceus,
from about 0.3 wt % to about 8 wt % by dry weight of Wolfiporia extensa,
from about 3 wt % to about 60 wt % by dry weight of Cordyceps militaris,
from about 0.3 wt % to about 8 wt % by dry weight of Fomes fomentarius,
from about 0.3 wt % to about 8 wt % by dry weight of Pleurotus ostreatus,
from about 0.3 wt % to about 8 wt % by dry weight of Phellinus linteus,
from about 0.3 wt % to about 8 wt % by dry weight of Grifola frondosa, and
from about 0.3 wt % to about 8 wt % by dry weight of Lentinula edodes.

46. The method of claim 42, wherein the wherein the psilocybin, the cannabidiol, and/or the dried mushroom blend are formulated together or separately.

47. The method of claim 46, wherein the psilocybin, the cannabidiol, and/or the dried mushroom blend are administered concurrently or substantially concurrently.

48. The method of claim 46, wherein the psilocybin, the cannabidiol, and/or the dried mushroom blend are administered within hours or days of one another.

49. The method of claim 33, wherein the maintenance dose further comprises niacin, piperine, turmeric, and/or omega fatty acid.

50. The method of claim 33, wherein the maintenance dose further comprises an agent selected from the group consisting of a serotonin reuptake inhibitor (SSRI), folic acid, St. John's Wart, dried Codiaeum variegatum, Codiaeum variegatum extract, fish oil, and flax seed oil.

51. The method of claim 33, wherein the subject has post-concussion syndrome.

52. The method of claim 33, wherein the subject has had a traumatic brain injury.

53. The method of claim 33, wherein the maintenance dose and/or loading dose comprises the mushroom Psilocybe cubensis.

54. A method for reducing a symptom of traumatic brain injury or post-concussion syndrome in a subject, the method comprising:
(i) administering to the subject at least one loading dose comprising between about 5 mg and about 30 mg psilocybin, inclusive; and
(ii) subsequently administering to the subject at least one maintenance dose comprising between about 1 mg to about 600 mg of a neurotransmitter activity modulator, and wherein the subject has been suspected or diagnosed as having traumatic brain injury or post-concussion syndrome.

55. The method of claim 54, wherein the symptom of traumatic brain injury or post-concussion syndrome comprises anxiety, depression, suicide ideation, stress, post-traumatic stress disorder, post-traumatic headache, progressive headache, feelings of dizziness, nausea, vomiting, noise sensitivity, being easily upset by loud noise, sleep disturbance, fatigue or tiring more easily, irritability, insomnia, nervousness, frustration or impatience, forgetfulness or poor memory, poor concentration, taking longer to think, blurred vision, light sensitivity, double vision, restlessness, insomnia, ringing in the ears, blurry vision, decrease in taste and/or smell, and various combinations thereof.

56. The method of any of claims 54-55, wherein the symptom of traumatic brain injury or post-concussion syndrome comprises anxiety and/or depression.

57. The method of any of claims 54-56, wherein the loading dose comprises about 5 mg to about 15 mg psilocybin.

58. The method of any of claims 54-57 wherein the loading dose comprises about 8 mg to about 12 mg psilocybin.

59. The method of any of claims 54-58, wherein the loading dose comprises about 25 mg psilocybin.

60. The method of any of claims 54-59, wherein the loading dose comprises about 30 mg psilocybin.

61. The method of any of claims 54-60, wherein the at least one maintenance dose further comprises between about 0.25 mg and about 0.3 mg psilocybin.

62. The method of any of claims 54-61, wherein the psilocybin of the loading dose comprises synthetic psilocybin.

63. The method of any of claims 54-62, wherein the psilocybin of the maintenance dose comprises synthetic psilocybin.

64. The method of any of claims 54-63, wherein the synthetic psilocybin is crystalline psilocybin.

65. The method of any of claims 54-64, wherein the synthetic psilocybin is amorphous psilocybin.

66. The method of any of claims 54-65, wherein the psilocybin is isolated from the mushroom Psilocybe cubensis.

67. The method of any of claims 54-66, wherein the neurotransmitter activity modulator comprises one or more selected from the group consisting of a tricyclic antidepressant (TCA), a selective serotonergic reuptake inhibitor (SSRI), a serotonin-norepinephrine reuptake inhibitor (SNRI), and a norepinephrine reuptake inhibitor (NRI).

68. The method of any of claims 54-67, wherein the neurotransmitter activity modulator comprises a tricyclic antidepressant (TCA).

69. The method of any of claims 54-67, wherein the neurotransmitter activity modulator comprises an SSRI.

70. The method of any of claims 54-67, wherein the neurotransmitter activity modulator comprises an SNRI.

71. The method of any of claims 54-67, wherein the neurotransmitter activity modulator comprises an NRI.

72. The method of any of claims 54-68, wherein the neurotransmitter activity modulator comprises imipramine.

73. The method of any of claims 54-72, wherein the loading dose is administered prior to an initial maintenance dose by at least seven days.

74. The method of any of claims 54-73, wherein two or more loading doses are administered over a 6 month time period.

75. The method of any of claims 54-74, wherein the maintenance dose is administered every day.

76. The method of any of claims 54-75, wherein the maintenance dose is not administered every day.

77. The method of any of claims 54-76, wherein the maintenance dose is administered according to a predetermined schedule.

78. A method for reducing a symptom of traumatic brain injury or post-concussion syndrome in a subject, the method comprising:
(i) administering to the subject at least one maintenance dose comprising between about 0.25 mg and about 0.3 mg psilocybin, inclusive; and
(ii) administering to the subject at least one maintenance dose comprising between about 1 mg to about 600 mg of CBD, inclusive, wherein the subject has been suspected or diagnosed as having traumatic brain injury or post-concussion syndrome.

79. The method of claim 78, wherein the symptom of traumatic brain injury or post-concussion syndrome comprises anxiety, depression, suicide ideation, stress, post-traumatic stress disorder, post-traumatic headache, progressive headache, feelings of dizziness, nausea, vomiting, noise sensitivity, being easily upset by loud noise, sleep disturbance, fatigue or tiring more easily, irritability, insomnia, nervousness, frustration or impatience, forgetfulness or poor memory, poor concentration, taking longer to think, blurred vision, light sensitivity, double vision, restlessness, insomnia, ringing in the ears, blurry vision, decrease in taste and/or smell, and various combinations thereof.

80. The method of any of claims 78-79, wherein the symptom of traumatic brain injury or post-concussion syndrome comprises anxiety or depression.

81. The method of any of claims 78-80, wherein the symptom of traumatic brain injury or post-concussion syndrome comprises anxiety.

82. The method of any of claims 78-81 wherein the symptom of traumatic brain injury or post-concussion syndrome comprises depression.

83. The method of any of claims 78-82, wherein the maintenance dose is administered every day

84. The method of any of claims 78-83, wherein the maintenance dose is administered according to a predetermined schedule.

85. The method of any of claims 78-84, wherein the loading dose comprises synthetic psilocybin.

86. The method of claim 85, wherein the synthetic psilocybin is crystalline psilocybin.

87. The method of claim 85, wherein the synthetic psilocybin is amorphous psilocybin.

88. The method of any of claims 78-84, wherein the psilocybin is isolated from the mushroom Psilocybe cubensis.

89. The method of any of claims 78-88, wherein the neurotransmitter activity modulator comprises one or more selected from the group consisting of a tricyclic antidepressant (TCA), a selective serotonergic reuptake inhibitor (SSRI), a serotonin-norepinephrine reuptake inhibitor (SNRI), and a norepinephrine reuptake inhibitor (NRI).

90. The method of any of claims 78-89, wherein the neurotransmitter activity modulator comprises a tricyclic antidepressant (TCA).

91. The method of any of claims 78-89, wherein the neurotransmitter activity modulator comprises an SSRI.

92. The method of any of claims 78-89, wherein the neurotransmitter activity modulator comprises an SNRI.

93. The method of any of claims 78-89, wherein the neurotransmitter activity modulator comprises an NRI.

94. The method of any of claims 78-89, wherein the neurotransmitter activity modulator comprises imipramine.

95. A composition comprising psilocybin and a neurotransmitter activity modulator from the group consisting of a tricyclic antidepressant, an SSRI, an SNRI, and an NRI.

96. The composition of claim 95, wherein the neurotransmitter activity modulator comprises a tricyclic antidepressant.

97. The composition of claim 95, wherein the neurotransmitter activity modulator comprises imipramine.

98. The composition of claim 95, wherein the neurotransmitter activity modulator comprises an SSRI.

99. The composition of claim 95, wherein the neurotransmitter activity modulator comprises an SNRI.

100. The composition of claim 95, wherein the neurotransmitter activity modulator comprises an NRI.

101. The composition of any of claims 95-100, wherein the neurotransmitter activity modulator comprises about 1 mg to about 600 mg of the neurotransmitter activity modulator.

102. The composition of claim 101, wherein the neurotransmitter activity modulator comprises about 1 mg to about 300 mg of the neurotransmitter activity modulator.

103. The composition of claim 101, wherein the neurotransmitter activity modulator comprises about 1 mg to about 200 mg of the neurotransmitter activity modulator.

104. The composition of claim 101, wherein the neurotransmitter activity modulator comprises about 1 mg to about 100 mg of the neurotransmitter activity modulator.

105. The composition of claim 101, wherein the neurotransmitter activity modulator comprises about 1 mg to about 50 mg of the neurotransmitter activity modulator.

106. The composition of claim 95, wherein the psilocybin comprises about 0.1 mg to about 0.5 mg.

107. The composition of claim 95, wherein the psilocybin comprises about 0.25 mg to about 0.3 mg.
